SEQUENCE LISTING (47 sequences)

SEQ ID NO: 1 - RNA, 21 nt, synthetic construct
  source 1..21: mol_type = other RNA
gttgctcgactctaaccgcta

SEQ ID NO: 2 - RNA, 23 nt, synthetic construct
  source 1..23: mol_type = other RNA
tagcggttagagtcgagcaacgg

SEQ ID NO: 3 - RNA, 21 nt, synthetic construct
  source 1..21: mol_type = other RNA
ttcgccaactattctacgtga

SEQ ID NO: 4 - RNA, 23 nt, synthetic construct
  source 1..23: mol_type = other RNA
tcacgtagaatagttggcgaagg

SEQ ID NO: 5 - RNA, 21 nt, synthetic construct
  source 1..21: mol_type = other RNA
accttcgccaactattctaca

SEQ ID NO: 6 - RNA, 23 nt, synthetic construct
  source 1..23: mol_type = other RNA
tgtagaatagttggcgaaggtct

SEQ ID NO: 7 - RNA, 21 nt, synthetic construct
  source 1..21: mol_type = other RNA
cgccaactattctacgtgcga

SEQ ID NO: 8 - RNA, 23 nt, synthetic construct
  source 1..23: mol_type = other RNA
tcgcacgtagaatagttggcgaa

SEQ ID NO: 9 - RNA, 21 nt, synthetic construct
  source 1..21: mol_type = other RNA
ccgcaagaggttctttaggga

SEQ ID NO: 10 - RNA, 23 nt, synthetic construct
  source 1..23: mol_type = other RNA
tccctaaagaacctcttgcgggt

SEQ ID NO: 11 - RNA, 21 nt, synthetic construct
  source 1..21: mol_type = other RNA
aaacgcctggtttcctactca

SEQ ID NO: 12 - RNA, 23 nt, synthetic construct
  source 1..23: mol_type = other RNA
tgagtaggaaaccaggcgtttca

SEQ ID NO: 13 - RNA, 21 nt, synthetic construct
  source 1..21: mol_type = other RNA
  modified_base 1: mod_base = OTHER; note = 2'-O-methylguanosine with a 3'-phosphorothioate linkage
  modified_base 2: mod_base = OTHER; note = 2'-O-methyluridine with a 3'-phosphorothioate linkage
  modified_base 3: mod_base = um
  modified_base 4: mod_base = gm
  modified_base 5: mod_base = cm
  modified_base 6: mod_base = um
  modified_base 7: mod_base = OTHER; note = 2'-fluorocytidine
  modified_base 8: mod_base = gm
  modified_base 9: mod_base = OTHER; note = 2'-fluoroadenosine
  modified_base 10: mod_base = OTHER; note = 2'-fluorocytidine
  modified_base 11: mod_base = OTHER; note = 2'-fluorouridine
  modified_base 12: mod_base = cm
  modified_base 13: mod_base = um
  modified_base 14: mod_base = OTHER; note = 2'-O-methyladenosine
  modified_base 15: mod_base = OTHER; note = 2'-O-methyladenosine
  modified_base 16: mod_base = cm
  modified_base 17: mod_base = cm
  modified_base 18: mod_base = gm
  modified_base 19: mod_base = OTHER; note = 2'-O-methylcytidine with a 3'-phosphorothioate linkage
  modified_base 20: mod_base = OTHER; note = 2'-O-methyluridine with a 3'-phosphorothioate linkage
  modified_base 21: mod_base = OTHER; note = 2'-O-methyladenosine
gttgctcgactctaaccgcta

SEQ ID NO: 14 - RNA, 23 nt, synthetic construct
  source 1..23: mol_type = other RNA
  modified_base 1: mod_base = OTHER; note = 2'-O-methyluridine with a 3'-phosphorothioate linkage and a 5'-vinylphosphonate substitution
  modified_base 2: mod_base = OTHER; note = 2'-fluoroadenosine with a 3'-phosphorothioate linkage
  modified_base 3: mod_base = gm
  modified_base 4: mod_base = cm
  modified_base 5: mod_base = gm
  modified_base 6: mod_base = OTHER; note = 2'-fluoroguanosine
  modified_base 7: mod_base = um
  modified_base 8: mod_base = um
  modified_base 9: mod_base = OTHER; note = 2'-O-methyladenosine
  modified_base 10: mod_base = gm
  modified_base 11: mod_base = OTHER; note = 2'-O-methyladenosine
  modified_base 12: mod_base = gm
  modified_base 13: mod_base = um
  modified_base 14: mod_base = OTHER; note = 2'-fluorocytidine
  modified_base 15: mod_base = gm
  modified_base 16: mod_base = OTHER; note = 2'-fluoroadenosine
  modified_base 17: mod_base = gm
  modified_base 18: mod_base = cm
  modified_base 19: mod_base = OTHER; note = 2'-O-methyladenosine
  modified_base 20: mod_base = OTHER; note = 2'-O-methyladenosine
  modified_base 21: mod_base = OTHER; note = 2'-O-methylcytidine with a 3'-phosphorothioate linkage
  modified_base 22: mod_base = OTHER; note = 2'-O-methylguanosine with a 3'-phosphorothioate linkage
  modified_base 23: mod_base = gm
tagcggttagagtcgagcaacgg

SEQ ID NO: 15 - RNA, 21 nt, synthetic construct
  source 1..21: mol_type = other RNA
  modified_base 1: mod_base = OTHER; note = 2'-O-methyluridine with a 3'-phosphorothioate linkage
  modified_base 2: mod_base = OTHER; note = 2'-O-methyluridine with a 3'-phosphorothioate linkage
  modified_base 3: mod_base = cm
  modified_base 4: mod_base = gm
  modified_base 5: mod_base = cm
  modified_base 6: mod_base = cm
  modified_base 7: mod_base = OTHER; note = 2'-fluoroadenosine
  modified_base 8: mod_base = OTHER; note = 2'-O-methyladenosine
  modified_base 9: mod_base = OTHER; note = 2'-fluorocytidine
  modified_base 10: mod_base = OTHER; note = 2'-fluorouridine
  modified_base 11: mod_base = OTHER; note = 2'-fluoroadenosine
  modified_base 12: mod_base = um
  modified_base 13: mod_base = um
  modified_base 14: mod_base = cm
  modified_base 15: mod_base = um
  modified_base 16: mod_base = OTHER; note = 2'-O-methyladenosine
  modified_base 17: mod_base = cm
  modified_base 18: mod_base = gm
  modified_base 19: mod_base = OTHER; note = 2'-O-methyluridine with a 3'-phosphorothioate linkage
  modified_base 20: mod_base = OTHER; note = 2'-O-methylguanosine with a 3'-phosphorothioate linkage
  modified_base 21: mod_base = OTHER; note = 2'-O-methyladenosine
ttcgccaactattctacgtga

SEQ ID NO: 16 - RNA, 23 nt, synthetic construct
  source 1..23: mol_type = other RNA
  modified_base 1: mod_base = OTHER; note = 2'-O-methyluridine with a 3'-phosphorothioate linkage and a 5'-vinylphosphonate substitution
  modified_base 2: mod_base = OTHER; note = 2'-fluorocytidine with a 3'-phosphorothioate linkage
  modified_base 3: mod_base = OTHER; note = 2'-O-methyladenosine
  modified_base 4: mod_base = cm
  modified_base 5: mod_base = gm
  modified_base 6: mod_base = OTHER; note = 2'-fluorouridine
  modified_base 7: mod_base = OTHER; note = 2'-O-methyladenosine
  modified_base 8: mod_base = gm
  modified_base 9: mod_base = OTHER; note = 2'-O-methyladenosine
  modified_base 10: mod_base = OTHER; note = 2'-O-methyladenosine
  modified_base 11: mod_base = um
  modified_base 12: mod_base = OTHER; note = 2'-O-methyladenosine
  modified_base 13: mod_base = gm
  modified_base 14: mod_base = OTHER; note = 2'-fluorouridine
  modified_base 15: mod_base = um
  modified_base 16: mod_base = OTHER; note = 2'-fluoroguanosine
  modified_base 17: mod_base = gm
  modified_base 18: mod_base = cm
  modified_base 19: mod_base = gm
  modified_base 20: mod_base = OTHER; note = 2'-O-methyladenosine
  modified_base 21: mod_base = OTHER; note = 2'-O-methyladenosine with a 3'-phosphorothioate linkage
  modified_base 22: mod_base = OTHER; note = 2'-O-methylguanosine with a 3'-phosphorothioate linkage
  modified_base 23: mod_base = gm
tcacgtagaatagttggcgaagg

SEQ ID NO: 17 - RNA, 21 nt, synthetic construct
  source 1..21: mol_type = other RNA
  modified_base 1: mod_base = OTHER; note = 2'-O-methyladenosine with a 3'-phosphorothioate linkage
  modified_base 2: mod_base = OTHER; note = 2'-O-methylcytidine with a 3'-phosphorothioate linkage
  modified_base 3: mod_base = cm
  modified_base 4: mod_base = um
  modified_base 5: mod_base = um
  modified_base 6: mod_base = cm
  modified_base 7: mod_base = OTHER; note = 2'-fluoroguanosine
  modified_base 8: mod_base = cm
  modified_base 9: mod_base = OTHER; note = 2'-fluorocytidine
  modified_base 10: mod_base = OTHER; note = 2'-fluoroadenosine
  modified_base 11: mod_base = OTHER; note = 2'-fluoroadenosine
  modified_base 12: mod_base = cm
  modified_base 13: mod_base = um
  modified_base 14: mod_base = OTHER; note = 2'-O-methyladenosine
  modified_base 15: mod_base = um
  modified_base 16: mod_base = um
  modified_base 17: mod_base = cm
  modified_base 18: mod_base = um
  modified_base 19: mod_base = OTHER; note = 2'-O-methyladenosine with a 3'-phosphorothioate linkage
  modified_base 20: mod_base = OTHER; note = 2'-O-methylcytidine with a 3'-phosphorothioate linkage
  modified_base 21: mod_base = OTHER; note = 2'-O-methyladenosine
accttcgccaactattctaca

SEQ ID NO: 18 - RNA, 23 nt, synthetic construct
  source 1..23: mol_type = other RNA
  modified_base 1: mod_base = OTHER; note = 2'-O-methyluridine with a 3'-phosphorothioate linkage and a 5'-vinylphosphonate substitution
  modified_base 2: mod_base = OTHER; note = 2'-fluoroguanosine with a 3'-phosphorothioate linkage
  modified_base 3: mod_base = um
  modified_base 4: mod_base = OTHER; note = 2'-O-methyladenosine
  modified_base 5: mod_base = gm
  modified_base 6: mod_base = OTHER; note = 2'-fluoroadenosine
  modified_base 7: mod_base = OTHER; note = 2'-O-methyladenosine
  modified_base 8: mod_base = um
  modified_base 9: mod_base = OTHER; note = 2'-O-methyladenosine
  modified_base 10: mod_base = gm
  modified_base 11: mod_base = um
  modified_base 12: mod_base = um
  modified_base 13: mod_base = gm
  modified_base 14: mod_base = OTHER; note = 2'-fluoroguanosine
  modified_base 15: mod_base = cm
  modified_base 16: mod_base = OTHER; note = 2'-fluoroguanosine
  modified_base 17: mod_base = OTHER; note = 2'-O-methyladenosine
  modified_base 18: mod_base = OTHER; note = 2'-O-methyladenosine
  modified_base 19: mod_base = gm
  modified_base 20: mod_base = gm
  modified_base 21: mod_base = OTHER; note = 2'-O-methyluridine with a 3'-phosphorothioate linkage
  modified_base 22: mod_base = OTHER; note = 2'-O-methylcytidine with a 3'-phosphorothioate linkage
  modified_base 23: mod_base = um
tgtagaatagttggcgaaggtct

SEQ ID NO: 19 - RNA, 21 nt, synthetic construct
  source 1..21: mol_type = other RNA
  modified_base 1: mod_base = OTHER; note = 2'-O-methylcytidine with a 3'-phosphorothioate linkage
  modified_base 2: mod_base = OTHER; note = 2'-O-methylguanosine with a 3'-phosphorothioate linkage
  modified_base 3: mod_base = cm
  modified_base 4: mod_base = cm
  modified_base 5: mod_base = OTHER; note = 2'-O-methyladenosine
  modified_base 6: mod_base = OTHER; note = 2'-O-methyladenosine
  modified_base 7: mod_base = OTHER; note = 2'-fluorocytidine
  modified_base 8: mod_base = um
  modified_base 9: mod_base = OTHER; note = 2'-fluoroadenosine
  modified_base 10: mod_base = OTHER; note = 2'-fluorouridine
  modified_base 11: mod_base = OTHER; note = 2'-fluorouridine
  modified_base 12: mod_base = cm
  modified_base 13: mod_base = um
  modified_base 14: mod_base = OTHER; note = 2'-O-methyladenosine
  modified_base 15: mod_base = cm
  modified_base 16: mod_base = gm
  modified_base 17: mod_base = um
  modified_base 18: mod_base = gm
  modified_base 19: mod_base = OTHER; note = 2'-O-methylcytidine with a 3'-phosphorothioate linkage
  modified_base 20: mod_base = OTHER; note = 2'-O-methylguanosine with a 3'-phosphorothioate linkage
  modified_base 21: mod_base = OTHER; note = 2'-O-methyladenosine
cgccaactattctacgtgcga

SEQ ID NO: 20 - RNA, 23 nt, synthetic construct
  source 1..23: mol_type = other RNA
  modified_base 1: mod_base = OTHER; note = 2'-O-methyluridine with a 3'-phosphorothioate linkage and a 5'-vinylphosphonate substitution
  modified_base 2: mod_base = OTHER; note = 2'-fluorocytidine with a 3'-phosphorothioate linkage
  modified_base 3: mod_base = gm
  modified_base 4: mod_base = cm
  modified_base 5: mod_base = OTHER; note = 2'-O-methyladenosine
  modified_base 6: mod_base = OTHER; note = 2'-fluorocytidine
  modified_base 7: mod_base = gm
  modified_base 8: mod_base = um
  modified_base 9: mod_base = OTHER; note = 2'-O-methyladenosine
  modified_base 10: mod_base = gm
  modified_base 11: mod_base = OTHER; note = 2'-O-methyladenosine
  modified_base 12: mod_base = OTHER; note = 2'-O-methyladenosine
  modified_base 13: mod_base = um
  modified_base 14: mod_base = OTHER; note = 2'-fluoroadenosine
  modified_base 15: mod_base = gm
  modified_base 16: mod_base = OTHER; note = 2'-fluorouridine
  modified_base 17: mod_base = um
  modified_base 18: mod_base = gm
  modified_base 19: mod_base = gm
  modified_base 20: mod_base = cm
  modified_base 21: mod_base = OTHER; note = 2'-O-methylguanosine with a 3'-phosphorothioate linkage
  modified_base 22: mod_base = OTHER; note = 2'-O-methyladenosine with a 3'-phosphorothioate linkage
  modified_base 23: mod_base = OTHER; note = 2'-O-methyladenosine
tcgcacgtagaatagttggcgaa

SEQ ID NO: 21 - RNA, 21 nt, synthetic construct
  source 1..21: mol_type = other RNA
  modified_base 1: mod_base = OTHER; note = 2'-O-methylcytidine with a 3'-phosphorothioate linkage
  modified_base 2: mod_base = OTHER; note = 2'-O-methylcytidine with a 3'-phosphorothioate linkage
  modified_base 3: mod_base = gm
  modified_base 4: mod_base = cm
  modified_base 5: mod_base = OTHER; note = 2'-O-methyladenosine
  modified_base 6: mod_base = OTHER; note = 2'-O-methyladenosine
  modified_base 7: mod_base = OTHER; note = 2'-fluoroguanosine
  modified_base 8: mod_base = OTHER; note = 2'-O-methyladenosine
  modified_base 9: mod_base = OTHER; note = 2'-fluoroguanosine
  modified_base 10: mod_base = OTHER; note = 2'-fluoroguanosine
  modified_base 11: mod_base = OTHER; note = 2'-fluorouridine
  modified_base 12: mod_base = um
  modified_base 13: mod_base = cm
  modified_base 14: mod_base = um
  modified_base 15: mod_base = um
  modified_base 16: mod_base = um
  modified_base 17: mod_base = OTHER; note = 2'-O-methyladenosine
  modified_base 18: mod_base = gm
  modified_base 19: mod_base = OTHER; note = 2'-O-methylguanosine with a 3'-phosphorothioate linkage
  modified_base 20: mod_base = OTHER; note = 2'-O-methylguanosine with a 3'-phosphorothioate linkage
  modified_base 21: mod_base = OTHER; note = 2'-O-methyladenosine
ccgcaagaggttctttaggga

SEQ ID NO: 22 - RNA, 23 nt, synthetic construct
  source 1..23: mol_type = other RNA
  modified_base 1: mod_base = OTHER; note = 2'-O-methyluridine with a 3'-phosphorothioate linkage and a 5'-vinylphosphonate substitution
  modified_base 2: mod_base = OTHER; note = 2'-fluorocytidine with a 3'-phosphorothioate linkage
  modified_base 3: mod_base = cm
  modified_base 4: mod_base = cm
  modified_base 5: mod_base = um
  modified_base 6: mod_base = OTHER; note = 2'-fluoroadenosine
  modified_base 7: mod_base = OTHER; note = 2'-O-methyladenosine
  modified_base 8: mod_base = OTHER; note = 2'-O-methyladenosine
  modified_base 9: mod_base = gm
  modified_base 10: mod_base = OTHER; note = 2'-O-methyladenosine
  modified_base 11: mod_base = OTHER; note = 2'-O-methyladenosine
  modified_base 12: mod_base = cm
  modified_base 13: mod_base = cm
  modified_base 14: mod_base = OTHER; note = 2'-fluorouridine
  modified_base 15: mod_base = cm
  modified_base 16: mod_base = OTHER; note = 2'-fluorouridine
  modified_base 17: mod_base = um
  modified_base 18: mod_base = gm
  modified_base 19: mod_base = cm
  modified_base 20: mod_base = gm
  modified_base 21: mod_base = OTHER; note = 2'-O-methylguanosine with a 3'-phosphorothioate linkage
  modified_base 22: mod_base = OTHER; note = 2'-O-methylguanosine with a 3'-phosphorothioate linkage
  modified_base 23: mod_base = um
tccctaaagaacctcttgcgggt

SEQ ID NO: 23 - RNA, 21 nt, synthetic construct
  source 1..21: mol_type = other RNA
  modified_base 1: mod_base = OTHER; note = 2'-O-methyladenosine with a 3'-phosphorothioate linkage
  modified_base 2: mod_base = OTHER; note = 2'-O-methyladenosine with a 3'-phosphorothioate linkage
  modified_base 3: mod_base = OTHER; note = 2'-O-methyladenosine
  modified_base 4: mod_base = cm
  modified_base 5: mod_base = gm
  modified_base 6: mod_base = cm
  modified_base 7: mod_base = OTHER; note = 2'-fluorocytidine
  modified_base 8: mod_base = um
  modified_base 9: mod_base = OTHER; note = 2'-fluoroguanosine
  modified_base 10: mod_base = OTHER; note = 2'-fluoroguanosine
  modified_base 11: mod_base = OTHER; note = 2'-fluorouridine
  modified_base 12: mod_base = um
  modified_base 13: mod_base = um
  modified_base 14: mod_base = cm
  modified_base 15: mod_base = cm
  modified_base 16: mod_base = um
  modified_base 17: mod_base = OTHER; note = 2'-O-methyladenosine
  modified_base 18: mod_base = cm
  modified_base 19: mod_base = OTHER; note = 2'-O-methyluridine with a 3'-phosphorothioate linkage
  modified_base 20: mod_base = OTHER; note = 2'-O-methylcytidine with a 3'-phosphorothioate linkage
  modified_base 21: mod_base = OTHER; note = 2'-O-methyladenosine
aaacgcctggtttcctactca

SEQ ID NO: 24 - RNA, 23 nt, synthetic construct
  source 1..23: mol_type = other RNA
  modified_base 1: mod_base = OTHER; note = 2'-O-methyluridine with a 3'-phosphorothioate linkage and a 5'-vinylphosphonate substitution
  modified_base 2: mod_base = OTHER; note = 2'-fluoroguanosine with a 3'-phosphorothioate linkage
  modified_base 3: mod_base = OTHER; note = 2'-O-methyladenosine
  modified_base 4: mod_base = gm
  modified_base 5: mod_base = um
  modified_base 6: mod_base = OTHER; note = 2'-fluoroadenosine
  modified_base 7: mod_base = gm
  modified_base 8: mod_base = gm
  modified_base 9: mod_base = OTHER; note = 2'-O-methyladenosine
  modified_base 10: mod_base = OTHER; note = 2'-O-methyladenosine
  modified_base 11: mod_base = OTHER; note = 2'-O-methyladenosine
  modified_base 12: mod_base = cm
  modified_base 13: mod_base = cm
  modified_base 14: mod_base = OTHER; note = 2'-fluoroadenosine
  modified_base 15: mod_base = gm
  modified_base 16: mod_base = OTHER; note = 2'-fluoroguanosine
  modified_base 17: mod_base = cm
  modified_base 18: mod_base = gm
  modified_base 19: mod_base = um
  modified_base 20: mod_base = um
  modified_base 21: mod_base = OTHER; note = 2'-O-methyluridine with a 3'-phosphorothioate linkage
  modified_base 22: mod_base = OTHER; note = 2'-O-methylcytidine with a 3'-phosphorothioate linkage
  modified_base 23: mod_base = OTHER; note = 2'-O-methyladenosine
tgagtaggaaaccaggcgtttca

SEQ ID NO: 25 - DNA, 10277 bp, Homo sapiens
  source 1..10277: mol_type = genomic DNA
atctcccagctcagccgagcccgtgcccaggccacgctttgttccagccgccgcctcctctaccctacggcgtccggagccatccctcgcctgctcgctctctcctttcgcccactccctgcatctgggcctgcatcacctttgccaaccgctcccccgatcctgccgacactcctcccccaaacttctgaccggcacccttgcctggtacccttctctccattcctccccctccatcttctttccccgacccctctcgggtccctcttttcccaaaacccgggtctctccgcgtggccccgcctccaggccggggatgtcccccgcggccccgcgcccatggtcctgacgctgcttctctccgcctacaagctgtgtcgcttcttcgccatgtcgggcccacggccgggcgccgagcggctggcggtgcctgggccagatgggggcggtggcacgggcccatggtgggctgcgggtggccgcgggccccgcgaagtgtcgccgggggcaggcaccgaggtgcaggacgccctggagcgcgcgctgccggagctgcagcaggccttgtccgcgctgaagcaggcgggcggcgcgcgggccgtgggcgccggcctggccgaggtcttccaactggtggaggaggcctggctgctgccggccgtgggccgcgaggtagcccagggtctgtgcgacgccatccgcctcgatggcggcctcgacctgctgttgcggctgctgcaggcgccggagttggagacgcgtgtgcaggccgcgcgcctgctggagcagatcctggtggctgagaaccgagaccgcgtggcgcgcattgggctgggcgtgatcctgaacctggcgaaggaacgcgaacccgtagagctggcgcggagcgtggcaggcatcttggagcacatgttcaagcattcggaggagacatgccagaggctggtggcggccggcggcctggacgcggtgctgtattggtgccgccgcacggaccccgcgctgctgcgccactgcgcgctggcgctgggcaactgcgcgctgcacgggggccaggcggtgcagcgacgcatggtagagaagcgcgcagccgagtggctcttcccgctcgccttctccaaggaggacgagctgcttcggctgcacgcctgcctcgcagtagcggtgttggcgactaacaaggaggtggagcgcgaggtggagcgctcgggcacgctggcgctcgtggagccgcttgtggcctcgctggaccctggccgcttcgcccgctgtctggtggacgccagcgacacaagccagggccgcgggcccgacgacctgcagcgcctcgtgccgttgctcgactctaaccgcttggaggcgcagtgcatcggggctttctacctctgcgccgaggctgccatcaagagcctgcaaggcaagaccaaggtgttcagcgacatcggcgccatccagagcctgaaacgcctggtttcctactctaccaatggcactaagtcggcgctggccaagcgcgcgctgcgcctgctgggcgaggaggtgccacggcccatcctgccctccgtgcccagctggaaggaggccgaggttcagacgtggctgcagcagatcggtttctccaagtactgcgagagcttccgggagcagcaggtggatggcgacctgcttctgcggctcacggaggaggaactccagaccgacctgggcatgaaatcgggcatcacccgcaagaggttctttagggagctcacggagctcaagaccttcgccaactattctacgtgcgaccgcagcaacctggcggactggctgggcagcctggacccgcgcttccgccagtacacctacggcctggtcagctgcggcctggaccgctccctgctgcaccgcgtgtctgagcagcagctgctggaagactgcggcatccacctgggcgtgcaccgcgcccgcatcctcacggcggccagagaaatgctacactccccgctgccctgtactggtggcaaacccagtggggacactccagatgtcttcatcagctaccgccggaactcaggttcccagctggccagtctcctgaaggtgcacctgcagctgcatggcttcagtgtcttcattgatgtggagaagctggaagcaggcaagttcgaggacaaactcatccagagtgtcatgggtgcccgcaactttgtgttggtgctatcacctggagcactggacaagtgcatgcaagaccatgactgcaaggattgggtgcataaggagattgtgactgctttaagctgcggcaagaacattgtgcccatcattgatggcttcgagtggcctgagccccaggtcctgcctgaggacatgcaggctgtgcttactttcaacggtatcaagtggtcccacgaataccaggaggccaccattgagaagatcatccgcttcctgcagggccgctcctcccgggactcatctgcaggctctgacaccagtttggagggtgctgcacccatgggtccaacctaaccagtccccagttccccagccctgctgtgacttccatttccatcgtcctttctgaaggaacagctcctgaaaccagtctccctgggctgagacaacctgggctcttcttaggaaatggctctccctccccctgtcccccaccctcatggcccacctccaacccactttcctcagtatctggagagggaagggaagtcaggcttgggcacgggaggttagaactcccccaggccctgccattgggttgtctgtctccgtcatggggagggtccctgctcagttctggagacactggagttggggtgggggtggttctgcattcccttctcctgctgatagcagtcagcttgaggaggatgacggaaggcagcctcagacaggaattaaggcaatgcccaggcgggcctgggcactgtattctgagcaagggcctgggcccaggagccagccagggatgagtgccatcatggctctccactcagactgtgcctggcccctgcacttacaacttcctgccgctctgtggccttgccctgtaatcactcagtgcccttagctagcctgactaagtcccagatcccctacagcttccttcggtgtggtatcttttgccacatccagggcgagggttgaggcaaaccagccctccctctgacttccttgtcactgcagccagctttgctgcacttgctggtgcacaggagcctcctgtttgggcctgggtctgggcatggggaggccgtgcctcaaagcccaccctaccccatgccttggtgctgtgcctcaggctccttcctggtctggcccagctggcttccccagcccctcagccatccagggctacccactgcttactcagggaccaggcagcccccatggcagtaaaagcagcctagacagaacctgcagctctgtggaaagaggcaaagtcctgaaaaggcaaagggttgtcacttagggcagcttctccaactttaacatgcatccaagtcacctgggaatgttgttaaaatcaggagatctggggtggggcctaggactctgcatttcttacagattcccaggtgagctgatgctggtggttaagggtagcaaatctctaaagcacgaagccctcacaaatctttgccatttcccaaacactccgctccatggtctccagtcatcagagcaactctacctggtattatcatccccattttacagataatgacactgaggctcagaaaggttgaggataagcccactttcctgtcattagtggcagccccagatccagacctaggcctcctggcacccagtccactggcagtggaattgctttcctgagaatcattctgaggctgggctattgcttctcccttgcttcaaagaatctagcagcgggggataggattttgcaacaaaaagctgacccagaggccatacagagcaggaatatcccattgccccctcctccactgggttcagagggtaagaaagcaccctccaataaacccaggctccaggccgtgggggctgctgaaggctctttccccgcaagggccaggtgttgacaccttaaagctggctgcgcccccagccccactcttggctgtgctggccaggtgactcctagttcttggccacatcatcagaaagtcaaaggtctcactccaggtttggggctccttccttccactcccctccctgccagagtctgtcttggccagtgccagcctcgatgctttggttttgaccccacctgatcctcctttcctcatgcagcacaagtgctcaccggggccagagccagggcatggatatgacaagcagggcagcctggacactgccctcacaggacagcgccaataacaatacagtgtctgagtatctccaggggatgatttctggctctttgtctccaatcagtcccactccctcctgaggtccccaagggcagtattcagagaggtttcctgcgttttatttctatttggtataccctccactgttgtccactgccctgtgtggccttctggttgacctctgcccgatcttctgtctctctgagggaatcagagtccagcatccagccccagctggaacagctgaagtcacaagcctcctctaagccaaggccagtgtgttcagaggtgactgccacccatactaggacaaacacagctcagatcaccaggtcaagcacctaggcctggcttctcctgagacagaggactcagaagtggcctttcctccaaagcctgctcagacacaggtctgtagggccagggtgttctgcttggctgggctgcagctgctacccctcggttggggctgagtcagccagatcctccccctacttctccccaagggccaagaactgctcagggacattaaaggtcaaaagtccagccacactcattcatcctttccccaggcccatgaagagaggcatctcattgtagaatgtatgaggaagtgggaagtatctcagagaatcagctaagtttcctaacttgtccatccaaatgtgatcaccacgattcaacaatttggggcattgctgatctagccgttcctagtggggcttgctcaaggttgcacagcgagtcagtagaagccctggctggccccacttggtaccaatccaccaggcagctcagggctcctgcccagcccagcagcttctgttgtctaacgtatggcaggcagactgggagcaggaaaacagagggccccaaagcccaaggcaccagaaggtttgtttcagtttgctgaagctgatttgtaatgattggcactcttcagccaggggagtgggtaggccatagccaaggatcgattccccaaccacagcaaaggcaacactcttcctccagagatcaccaagcccctcttacctccctccctccttcccaaggctggcactaaccaggtaccacattcattgttaaggaatggctgatgactgctacacgtgttgggaacctggttggggctgtgcagtttgggctggaaggagagatgccagccctcgtgctgcctctggtccctgaagtgtcacctctctcaggacctctcctctggcctgtggggttataagtgatggatagcagaaagggagaactgactcctgtcccaaatagctcctctgccacctgtcctgcagtgggcctgtgtgggttatgattctagatcctagacagaggctgggtcagctgtggatggggtggtgccttggtctctcttgactacctcgtccaaagagagcactgcccttagacaagagttgcttgtcctgctgtgggctgggcttccagctgcagacctccagttgcttggtgttcactttgctcctcttgccctctgtcttctggtccaggcagatcaggggctctggggaaactgctggaactcgaggtgaggatcagccttttccagcatcctgtgagagaccagagagagagtttggatttcatgtggggaaccctcaaggcctgtctggagaagtgacacaggatttactggggtgggctggtccaggtagctctcctgaacctcctccttccccaagctgagaagctgagagctggaggacaatatccagggacatggctctggaaaataacttttttttttttaagagacagggtcttgctctgttgtccaggctggagggcagtgacataatcatagctcactgtacccttgaactcctgggctcaagtgatcctcctgcctcagcctccttagtagctgggactaccagtgcataccaccatgcctgggtgattttttaaattttttatacagacaaggtcttgctatgttgcccaggctgatcttgaattcccgggctcaagtggtcctcctgcctcagcctcccacaggatcgggattacaggcaagagcctccacgcccggccatgaaatataattcttaatatcatacaggaaaaagtcagcgggtcaagctagcctgtggcccagccacaactagctgacaaagcttcctggccttccctttaacacagttctgctgccatagttccatctataaaatgggaatggagggaaataggggaactgggagagagaacacagccttgccaagcagcaatgttagcctgatccttcctccacctagctcgccatctcgcccttggaaaatggctcctggaggattaggcagccatctgcaaggagaggggcaacctgggacaagacacccagagggtaaggattccaggaatgaagctgccatttctggttgggaggagaagaggaaacttttaagagaaagggctccattatgagcatgggttcagggccctgcattacccaatcagaacagccgggatgagcaggaggccagctcccaggaggaaggggaaccccttcataaagttcagagtggctgggtagagtgagttgaagatgccggaggccgtcagcatggccaggctattcacacaggccacagcagaaaagagagcacctgtgaagaaataaataccatactctggagtccgaaagggccatattccaactctggcaccaccacctcacagctgtgtgaccgggagtagtcacttaacctatgtctccccttcctcaccagtaaatcctgctacatcatgtactgtgacaaggattcagtaaggtcatatgtggacagtagctggcacagaggggctactaaacaaatggctgctattaaatccacattaaaagtacatgtgatctgacagaacccagcacataaaagaaaaaaaaagtacatgtgatattgtctgatgaaagcttgatggaaatggcttttttctggtttatcctctttggaatcatctcctgtttgggattaactgctggtctgatcagttccaatattcatagcggtgtcaccactgaatagcttcttatcctttgggttcctgttcctccttctgctaaataaggataatacctatttcctagattgtgagcaacattaagttcacatggaaatcacccatcactgggcctggtcccctggaagtagctagttagtaagggctgttcttttctcctgtttctcttgacatctctgggcacagagaaagtgctgggaaaaaaagtttaggtgaatgaatgaagacacatggattctggggacaccagaacccacagtgggctctgtatggcaccagagtctctgtcatcatcagatcctcattccaggacagatggaaaaagatgaatgtttccagactggggcataaagacccagaggctggagaagctgttctttatagatataccaggagaacccacagtttacaaaatgtgcaacaacccaacagaagttgagattaaattctgtcacatctagaggggtctgtgatgtcatcaaaagcaaaccacccacatcacagatgaagaaacaggcctgtggcagggctcggactaaaacccagatcctgagaccagctgcttttaaacacagacgtaggtttgcatcctagctccaccatttactgagtaaccttgggtgagccaatgtaaccccctgggtctctgtttctttatctgtcaactgtggaaaatgaaacccatgtcacaaggttgttcacttctgggcttgtacacgctgaccccagagaaacagggaactctggcatcaccacacccatcttacagacggaaaagctgaggtctgcagagagtaaatcctctgctctggttatctagaaagaacataattgtgctctgctgactgcaaatcccaactctgcggtttgaaaatccaaggtggcatgatcctctgcccattgtgggcaatttcacagaaatgtgtttgttttggccacttacttctccagggtgagaggggggaaggcaagctgttcccccagccatggctgcccatcagcccgtttcgggcagcactggacatgaggaaccagacacaggtgggttctgacactcaccctgctctgtctctctcaccagcttggagagtttagcccggatgacaggtgtgatgactaatgacaggaaaagcaacccatatcctgtggagaaacaaacactcatcaggaaaatggggctggggagaggggcgtccaagggaaaggcagcagagctcctatccataccccacgtggggcttaggttagacccaggaagaacttccttgatggtgagggtgggaagacagtagtcaaggaggaatggagactgcccttgtctgggcttggccacctgctagctctcatgaatgaatgctaattcccattgattgctttcttgtctgaacctcttgtggtcacagcaggcatcacccacccacttggcacttagtagggatatggcagggcacagaaaacaagcatgggctttggagtcagccctgagttcaaaacctgatgccattacatattatctgtgtggcctggggtacttaccctctctgatcctgactccctgtatgaggaagataataaggccttcatcacaggatggttctgaggcataggaggctgaataatggtgcccaatggcatcagattcatagccctggaacctgtaaatactaccttatttggaaaatgagtctatgcaggtgtgcagttaagcctcctgagagagcagagttatcctggattaggttgggccctaaatgccgtcacacatatctttataagaggaaagcagacggagatttggcaccgacagaattgagaaggcacaaagaggaggagagtcaatgtgagcacagaggcagagactggtgatggccgccccaagccaaggaatgccagcagccccagaagctggaagaaatgagaaacacgttctctcctggaggcttgcaagggagcactgcctgctgactgcttccattcagcccggtggtactgactttggacttctggcctccagaactgtgagagaatatgtttctgttgtgttaagcccccaagtttgtggtatgtcattacagcaatctcagggaaccaatacatgaggtaaaaaggtaacatctatgaagagcatggcatagggacacagcaaatgggagttccttttccctttgcattcagttacttacaggcttcctgttttcttcataaccatttctctccctgtgcgactgctgactcctcagcaaaactgcaaactcctacaggacagtggatcctccaaagaaggtatacgatgaggcatccagggaccctagcagtgtcaggcccctcaaatcccactctgttgagacctccccccgacccagagcaatgacagcatctttatcatctctgcatcccccagggccatcagcaggagggaaaggttcccttctgcttaattgtcagacaagcagttgagttaagaaatctgtgattattgtattgttgactatacacagcacattttagggctctatcaaaataaatctgtccctttaaaaaaagttaactaaagccgggcacggtggctcatgcctgtaatcccaacactttgggaggctgaggcaggcggatccttgagctcaggagttagagacctggactgggcaaaatggtgaggaccccatctctataaaaaatacaaaaattagcaaggtgtggtaatgtgcaccagtggtcccagctactagagaggccaaggtgggaggatcatctgggcccgggggatgaggctgcagtgagccatgatcgtgccactgcactctagcctgggtaacaaagcgagaccctgtctctaaatacatcaatcaaataaaaattttaaaaagttaa

SEQ ID NO: 26 - protein, 724 aa, Homo sapiens
MVLTLLLSAYKLCRFFAMSGPRPGAERLAVPGPDGGGGTGPWWAAGGRGPREVSPGAGTEVQDALERALPELQQALSALKQAGGARAVGAGLAEVFQLVEEAWLLPAVGREVAQGLCDAIRLDGGLDLLLRLLQAPELETRVQAARLLEQILVAENRDRVARIGLGVILNLAKEREPVELARSVAGILEHMFKHSEETCQRLVAAGGLDAVLYWCRRTDPALLRHCALALGNCALHGGQAVQRRMVEKRAAEWLFPLAFSKEDELLRLHACLAVAVLATNKEVEREVERSGTLALVEPLVASLDPGRFARCLVDASDTSQGRGPDDLQRLVPLLDSNRLEAQCIGAFYLCAEAAIKSLQGKTKVFSDIGAIQSLKRLVSYSTNGTKSALAKRALRLLGEEVPRPILPSVPSWKEAEVQTWLQQIGFSKYCESFREQQVDGDLLLRLTEEELQTDLGMKSGITRKRFFRELTELKTFANYSTCDRSNLADWLGSLDPRFRQYTYGLVSCGLDRSLLHRVSEQQLLEDCGIHLGVHRARILTAAREMLHSPLPCTGGKPSGDTPDVFISYRRNSGSQLASLLKVHLQLHGFSVFIDVEKLEAGKFEDKLIQSVMGARNFVLVLSPGALDKCMQDHDCKDWVHKEIVTALSCGKNIVPIIDGFEWPEPQVLPEDMQAVLTFNGIKWSHEYQEATIEKIIRFLQGRSSRDSSAGSDTSLEGAAPMGPT

SEQ ID NO: 27 - RNA, 23 nt, synthetic construct
  source 1..23: mol_type = other RNA
  modified_base 1: mod_base = OTHER; note = 2'-O-methyluridine with a 3'-phosphorothioate linkage
  modified_base 2: mod_base = OTHER; note = 2'-fluorocytidine with a 3'-phosphorothioate linkage
  modified_base 3: mod_base = OTHER; note = 2'-O-methyladenosine
  modified_base 4: mod_base = cm
  modified_base 5: mod_base = gm
  modified_base 6: mod_base = OTHER; note = 2'-fluorouridine
  modified_base 7: mod_base = OTHER; note = 2'-O-methyladenosine
  modified_base 8: mod_base = gm
  modified_base 9: mod_base = OTHER; note = 2'-O-methyladenosine
  modified_base 10: mod_base = OTHER; note = 2'-O-methyladenosine
  modified_base 11: mod_base = um
  modified_base 12: mod_base = OTHER; note = 2'-O-methyladenosine
  modified_base 13: mod_base = gm
  modified_base 14: mod_base = OTHER; note = 2'-fluorouridine
  modified_base 15: mod_base = um
  modified_base 16: mod_base = OTHER; note = 2'-fluoroguanosine
  modified_base 17: mod_base = gm
  modified_base 18: mod_base = cm
  modified_base 19: mod_base = gm
  modified_base 20: mod_base = OTHER; note = 2'-O-methyladenosine
  modified_base 21: mod_base = OTHER; note = 2'-O-methyladenosine with a 3'-phosphorothioate linkage
  modified_base 22: mod_base = OTHER; note = 2'-O-methylguanosine with a 3'-phosphorothioate linkage
  modified_base 23: mod_base = gm
tcacgtagaatagttggcgaagg

SEQ ID NO: 28 - RNA, 21 nt, synthetic construct
  source 1..21: mol_type = other RNA
  modified_base 1: mod_base = OTHER; note = 2'-O-methyluridine with a 3'-phosphorothioate linkage
  modified_base 2: mod_base = OTHER; note = 2'-O-methyluridine with a 3'-phosphorothioate linkage
  modified_base 3: mod_base = cm
  modified_base 4: mod_base = gm
  modified_base 5: mod_base = cm
  modified_base 6: mod_base = cm
  modified_base 7: mod_base = OTHER; note = 2'-O-methyladenosine
  modified_base 8: mod_base = OTHER; note = 2'-O-methyladenosine
  modified_base 9: mod_base = OTHER; note = 2'-fluorocytidine
  modified_base 10: mod_base = OTHER; note = 2'-fluorouridine
  modified_base 11: mod_base = OTHER; note = 2'-fluoroadenosine
  modified_base 12: mod_base = um
  modified_base 13: mod_base = um
  modified_base 14: mod_base = cm
  modified_base 15: mod_base = um
  modified_base 16: mod_base = OTHER; note = 2'-O-methyladenosine
  modified_base 17: mod_base = cm
  modified_base 18: mod_base = gm
  modified_base 19: mod_base = OTHER; note = 2'-O-methyluridine with a 3'-phosphorothioate linkage
  modified_base 20: mod_base = OTHER; note = 2'-O-methylguanosine with a 3'-phosphorothioate linkage
  modified_base 21: mod_base = OTHER; note = 2'-O-methyladenosine
ttcgccaactattctacgtga

SEQ ID NO: 29 - RNA, 23 nt, synthetic construct
  source 1..23: mol_type = other RNA
  modified_base 1: mod_base = OTHER; note = 2'-O-methyluridine with a 3'-phosphorothioate linkage
  modified_base 2: mod_base = OTHER; note = 2'-fluorocytidine with a 3'-phosphorothioate linkage
  modified_base 3: mod_base = OTHER; note = 2'-O-methyladenosine
  modified_base 4: mod_base = cm
  modified_base 5: mod_base = OTHER; note = 2'-fluoroguanosine
  modified_base 6: mod_base = um
  modified_base 7: mod_base = OTHER; note = 2'-fluoroadenosine
  modified_base 8: mod_base = gm
  modified_base 9: mod_base = OTHER; note = 2'-O-methyladenosine
  modified_base 10: mod_base = OTHER; note = 2'-O-methyladenosine
  modified_base 11: mod_base = um
  modified_base 12: mod_base = OTHER; note = 2'-O-methyladenosine
  modified_base 13: mod_base = gm
  modified_base 14: mod_base = OTHER; note = 2'-fluorouridine
  modified_base 15: mod_base = um
  modified_base 16: mod_base = OTHER; note = 2'-fluoroguanosine
  modified_base 17: mod_base = gm
  modified_base 18: mod_base = cm
  modified_base 19: mod_base = gm
  modified_base 20: mod_base = OTHER; note = 2'-O-methyladenosine
  modified_base 21: mod_base = OTHER; note = 2'-O-methyladenosine with a 3'-phosphorothioate linkage
  modified_base 22: mod_base = OTHER; note = 2'-O-methylguanosine with a 3'-phosphorothioate linkage
  modified_base 23: mod_base = gm
tcacgtagaatagttggcgaagg

SEQ ID NO: 30 - RNA, 23 nt, synthetic construct
  source 1..23: mol_type = other RNA
  modified_base 1: mod_base = OTHER; note = 2'-O-methyluridine with a 3'-phosphorothioate linkage
  modified_base 2: mod_base = OTHER; note = 2'-fluorocytidine with a 3'-phosphorothioate linkage
  modified_base 3: mod_base = OTHER; note = 2'-fluoroadenosine
  modified_base 4: mod_base = cm
  modified_base 5: mod_base = gm
  modified_base 6: mod_base = um
  modified_base 7: mod_base = OTHER; note = 2'-fluoroadenosine
  modified_base 8: mod_base = gm
  modified_base 9: mod_base = OTHER; note = 2'-O-methyladenosine
  modified_base 10: mod_base = OTHER; note = 2'-O-methyladenosine
  modified_base 11: mod_base = um
  modified_base 12: mod_base = OTHER; note = 2'-O-methyladenosine
  modified_base 13: mod_base = gm
  modified_base 14: mod_base = OTHER; note = 2'-fluorouridine
  modified_base 15: mod_base = um
  modified_base 16: mod_base = OTHER; note = 2'-fluoroguanosine
  modified_base 17: mod_base = gm
  modified_base 18: mod_base = cm
  modified_base 19: mod_base = gm
  modified_base 20: mod_base = OTHER; note = 2'-O-methyladenosine
  modified_base 21: mod_base = OTHER; note = 2'-O-methyladenosine with a 3'-phosphorothioate linkage
  modified_base 22: mod_base = OTHER; note = 2'-O-methylguanosine with a 3'-phosphorothioate linkage
  modified_base 23: mod_base = gm
tcacgtagaatagttggcgaagg

SEQ ID NO: 31 - RNA, 23 nt, synthetic construct
  source 1..23: mol_type = other RNA
  modified_base 1: mod_base = OTHER; note = 2'-O-methyluridine with a 3'-phosphorothioate linkage
  modified_base 2: mod_base = OTHER; note = 2'-fluorocytidine with a 3'-phosphorothioate linkage
  modified_base 3: mod_base = OTHER; note = 2'-O-methyladenosine
  modified_base 4: mod_base = cm
  modified_base 5: mod_base = OTHER; note = 2'-fluoroguanosine
  modified_base 6: mod_base = um
  modified_base 7: mod_base = OTHER; note = 2'-O-methyladenosine
  modified_base 8: mod_base = OTHER; note = 2'-fluoroguanosine
  modified_base 9: mod_base = OTHER; note = 2'-O-methyladenosine
  modified_base 10: mod_base = OTHER; note = 2'-O-methyladenosine
  modified_base 11: mod_base = um
  modified_base 12: mod_base = OTHER; note = 2'-O-methyladenosine
  modified_base 13: mod_base = gm
  modified_base 14: mod_base = OTHER; note = 2'-fluorouridine
  modified_base 15: mod_base = um
  modified_base 16: mod_base = OTHER; note = 2'-fluoroguanosine
  modified_base 17: mod_base = gm
  modified_base 18: mod_base = cm
  modified_base 19: mod_base = gm
  modified_base 20: mod_base = OTHER; note = 2'-O-methyladenosine
  modified_base 21: mod_base = OTHER; note = 2'-O-methyladenosine with a 3'-phosphorothioate linkage
  modified_base 22: mod_base = OTHER; note = 2'-O-methylguanosine with a 3'-phosphorothioate linkage
  modified_base 23: mod_base = gm
tcacgtagaatagttggcgaagg

SEQ ID NO: 32 - RNA, 23 nt, synthetic construct
  source 1..23: mol_type = other RNA
  modified_base 1: mod_base = OTHER; note = 2'-O-methyluridine with a 3'-phosphorothioate linkage
  modified_base 2: mod_base = OTHER; note = 2'-fluorocytidine with a 3'-phosphorothioate linkage
  modified_base 3: mod_base = OTHER; note = 2'-O-methyladenosine
  modified_base 4: mod_base = cm
  modified_base 5: mod_base = gm
  modified_base 6: mod_base = um
  modified_base 7: mod_base = OTHER; note = 2'-O-methyladenosine
  modified_base 8: mod_base = gm
  modified_base 9: mod_base = OTHER; note = 2'-O-methyladenosine
  modified_base 10: mod_base = OTHER; note = 2'-O-methyladenosine
  modified_base 11: mod_base = um
  modified_base 12: mod_base = OTHER; note = 2'-O-methyladenosine
  modified_base 13: mod_base = gm
  modified_base 14: mod_base = OTHER; note = 2'-fluorouridine
  modified_base 15: mod_base = um
  modified_base 16: mod_base = gm
  modified_base 17: mod_base = gm
  modified_base 18: mod_base = cm
  modified_base 19: mod_base = gm
  modified_base 20: mod_base = OTHER; note = 2'-O-methyladenosine
  modified_base 21: mod_base = OTHER; note = 2'-O-methyladenosine with a 3'-phosphorothioate linkage
  modified_base 22: mod_base = OTHER; note = 2'-O-methylguanosine with a 3'-phosphorothioate linkage
  modified_base 23: mod_base = gm
tcacgtagaatagttggcgaagg

SEQ ID NO: 33 - RNA, 23 nt, synthetic construct
  source 1..23: mol_type = other RNA
  modified_base 1: mod_base = OTHER; note = 2'-O-methyluridine with a 3'-phosphorothioate linkage
  modified_base 2: mod_base = OTHER; note = 2'-fluoroadenosine with a 3'-phosphorothioate linkage
  modified_base 3: mod_base = gm
  modified_base 4: mod_base = cm
  modified_base 5: mod_base = gm
  modified_base 6: mod_base = OTHER; note = 2'-fluoroguanosine
  modified_base 7: mod_base = um
  modified_base 8: mod_base = um
  modified_base 9: mod_base = OTHER; note = 2'-O-methyladenosine
  modified_base 10: mod_base = gm
  modified_base 11: mod_base = OTHER; note = 2'-O-methyladenosine
  modified_base 12: mod_base = gm
  modified_base 13: mod_base = um
  modified_base 14: mod_base = OTHER; note = 2'-fluorocytidine
  modified_base 15: mod_base = gm
  modified_base 16: mod_base = OTHER; note = 2'-fluoroadenosine
  modified_base 17: mod_base = gm
  modified_base 18: mod_base = cm
  modified_base 19: mod_base = OTHER; note = 2'-O-methyladenosine
  modified_base 20: mod_base = OTHER; note = 2'-O-methyladenosine
  modified_base 21: mod_base = OTHER; note = 2'-O-methylcytidine with a 3'-phosphorothioate linkage
  modified_base 22: mod_base = OTHER; note = 2'-O-methylguanosine with a 3'-phosphorothioate linkage
  modified_base 23: mod_base = gm
tagcggttagagtcgagcaacgg

SEQ ID NO: 34 - RNA, 21 nt, synthetic construct
  source 1..21: mol_type = other RNA
  modified_base 1: mod_base = OTHER; note = 2'-O-methylguanosine with a 3'-phosphorothioate linkage
  modified_base 2: mod_base = OTHER; note = 2'-O-methyluridine with a 3'-phosphorothioate linkage
  modified_base 3: mod_base = um
  modified_base 4: mod_base = gm
  modified_base 5: mod_base = cm
  modified_base 6: mod_base = um
  modified_base 7: mod_base = cm
  modified_base 8: mod_base = gm
  modified_base 9: mod_base = OTHER; note = 2'-fluoroadenosine
  modified_base 10: mod_base = OTHER; note = 2'-fluorocytidine
  modified_base 11: mod_base = OTHER; note = 2'-fluorouridine
  modified_base 12: mod_base = cm
  modified_base 13: mod_base = um
  modified_base 14: mod_base = OTHER; note = 2'-O-methyladenosine
  modified_base 15: mod_base = OTHER; note = 2'-O-methyladenosine
  modified_base 16: mod_base = cm
  modified_base 17: mod_base = cm
  modified_base 18: mod_base = gm
  modified_base 19: mod_base = OTHER; note = 2'-O-methylcytidine with a 3'-phosphorothioate linkage
  modified_base 20: mod_base = OTHER; note = 2'-O-methyluridine with a 3'-phosphorothioate linkage
  modified_base 21: mod_base = OTHER; note = 2'-O-methyladenosine
gttgctcgactctaaccgcta

SEQ ID NO: 35 - RNA, 23 nt, synthetic construct
  source 1..23: mol_type = other RNA
  modified_base 1: mod_base = OTHER; note = 2'-O-methyluridine with a 3'-phosphorothioate linkage
  modified_base 2: mod_base = OTHER; note = 2'-fluoroadenosine with a 3'-phosphorothioate linkage
  modified_base 3: mod_base = gm
  modified_base 4: mod_base = cm
  modified_base 5: mod_base = OTHER; note = 2'-fluoroguanosine
  modified_base 6: mod_base = gm
  modified_base 7: mod_base = OTHER; note = 2'-fluorouridine
  modified_base 8: mod_base = um
  modified_base 9: mod_base = OTHER; note = 2'-O-methyladenosine
  modified_base 10: mod_base = gm
  modified_base 11: mod_base = OTHER; note = 2'-O-methyladenosine
  modified_base 12: mod_base = gm
  modified_base 13: mod_base = um
  modified_base 14: mod_base = OTHER; note = 2'-fluorocytidine
  modified_base 15: mod_base = gm
  modified_base 16: mod_base = OTHER; note = 2'-fluoroadenosine
  modified_base 17: mod_base = gm
  modified_base 18: mod_base = cm
  modified_base 19: mod_base = OTHER; note = 2'-O-methyladenosine
  modified_base 20: mod_base = OTHER; note = 2'-O-methyladenosine
  modified_base 21: mod_base = OTHER; note = 2'-O-methylcytidine with a 3'-phosphorothioate linkage
  modified_base 22: mod_base = OTHER; note = 2'-O-methylguanosine with a 3'-phosphorothioate linkage
  modified_base 23: mod_base = gm
tagcggttagagtcgagcaacgg

SEQ ID NO: 36 - RNA, 23 nt, synthetic construct
  source 1..23: mol_type = other RNA
  modified_base 1: mod_base = OTHER; note = 2'-O-methyluridine with a 3'-phosphorothioate linkage
  modified_base 2: mod_base = OTHER; note = 2'-fluoroadenosine with a 3'-phosphorothioate linkage
  modified_base 3: mod_base = OTHER; note = 2'-fluoroguanosine
  modified_base 4: mod_base = cm
  modified_base 5: mod_base = gm
  modified_base 6: mod_base = gm
  modified_base 7: mod_base = OTHER; note = 2'-fluorouridine
  modified_base 8: mod_base = um
  modified_base 9: mod_base = OTHER; note = 2'-O-methyladenosine
  modified_base 10: mod_base = gm
  modified_base 11: mod_base = OTHER; note = 2'-O-methyladenosine
  modified_base 12: mod_base = gm
  modified_base 13: mod_base = um
  modified_base 14: mod_base = OTHER; note = 2'-fluorocytidine
  modified_base 15: mod_base = gm
  modified_base 16: mod_base = OTHER; note = 2'-fluoroadenosine
  modified_base 17: mod_base = gm
  modified_base 18: mod_base = cm
  modified_base 19: mod_base = OTHER; note = 2'-O-methyladenosine
  modified_base 20: mod_base = OTHER; note = 2'-O-methyladenosine
  modified_base 21: mod_base = OTHER; note = 2'-O-methylcytidine with a 3'-phosphorothioate linkage
  modified_base 22: mod_base = OTHER; note = 2'-O-methylguanosine with a 3'-phosphorothioate linkage
  modified_base 23: mod_base = gm
tagcggttagagtcgagcaacgg

SEQ ID NO: 37 - RNA, 23 nt, synthetic construct
  source 1..23: mol_type = other RNA
  modified_base 1: mod_base = OTHER; note = 2'-O-methyluridine with a 3'-phosphorothioate linkage
  modified_base 2: mod_base = OTHER; note = 2'-fluoroadenosine with a 3'-phosphorothioate linkage
  modified_base 3: mod_base = gm
  modified_base 4: mod_base = cm
  modified_base 5: mod_base = OTHER; note = 2'-fluoroguanosine
  modified_base 6: mod_base = gm
  modified_base 7: mod_base = um
  modified_base 8: mod_base = OTHER; note = 2'-fluorouridine
  modified_base 9: mod_base = OTHER; note = 2'-O-methyladenosine
  modified_base 10: mod_base = gm
  modified_base 11: mod_base = OTHER; note = 2'-O-methyladenosine
  modified_base 12: mod_base = gm
  modified_base 13: mod_base = um
  modified_base 14: mod_base = OTHER; note = 2'-fluorocytidine
  modified_base 15: mod_base = gm
  modified_base 16: mod_base = OTHER; note = 2'-fluoroadenosine
  modified_base 17: mod_base = gm
  modified_base 18: mod_base = cm
  modified_base 19: mod_base = OTHER; note = 2'-O-methyladenosine
  modified_base 20: mod_base = OTHER; note = 2'-O-methyladenosine
  modified_base 21: mod_base = OTHER; note = 2'-O-methylcytidine with a 3'-phosphorothioate linkage
  modified_base 22: mod_base = OTHER; note = 2'-O-methylguanosine with a 3'-phosphorothioate linkage
  modified_base 23: mod_base = gm
tagcggttagagtcgagcaacgg

SEQ ID NO: 38 - RNA, 23 nt, synthetic construct
  source 1..23: mol_type = other RNA
  modified_base 1: mod_base = OTHER; note = 2'-O-methyluridine with a 3'-phosphorothioate linkage
  modified_base 2: mod_base = OTHER; note = 2'-fluoroadenosine with a 3'-phosphorothioate linkage
  modified_base 3: mod_base = gm
  modified_base 4: mod_base = cm
  modified_base 5: mod_base = gm
  modified_base 6: mod_base = gm
  modified_base 7: mod_base = um
  modified_base 8: mod_base = um
  modified_base 9: mod_base = OTHER; note = 2'-O-methyladenosine
  modified_base 10: mod_base = gm
  modified_base 11: mod_base = OTHER; note = 2'-O-methyladenosine
  modified_base 12: mod_base = gm
  modified_base 13: mod_base = um
  modified_base 14: mod_base = OTHER; note = 2'-fluorocytidine
  modified_base 15: mod_base = gm
  modified_base 16: mod_base = OTHER; note = 2'-O-methyladenosine
  modified_base 17: mod_base = gm
  modified_base 18: mod_base = cm
  modified_base 19: mod_base = OTHER; note = 2'-O-methyladenosine
  modified_base 20: mod_base = OTHER; note = 2'-O-methyladenosine
  modified_base 21: mod_base = OTHER; note = 2'-O-methylcytidine with a 3'-phosphorothioate linkage
  modified_base 22: mod_base = OTHER; note = 2'-O-methylguanosine with a 3'-phosphorothioate linkage
  modified_base 23: mod_base = gm
tagcggttagagtcgagcaacgg

SEQ ID NO: 39 - RNA, 23 nt, synthetic construct
  source 1..23: mol_type = other RNA
  modified_base 1: mod_base = OTHER; note = 2'-O-methyluridine with a 3'-phosphorothioate linkage
  modified_base 2: mod_base = OTHER; note = 2'-fluorocytidine with a 3'-phosphorothioate linkage
  modified_base 3: mod_base = cm
  modified_base 4: mod_base = cm
  modified_base 5: mod_base = um
  modified_base 6: mod_base = OTHER; note = 2'-fluoroadenosine
  modified_base 7: mod_base = OTHER; note = 2'-O-methyladenosine
  modified_base 8: mod_base = OTHER; note = 2'-O-methyladenosine
  modified_base 9: mod_base = gm
  modified_base 10: mod_base = OTHER; note = 2'-O-methyladenosine
  modified_base 11: mod_base = OTHER; note = 2'-O-methyladenosine
  modified_base 12: mod_base = cm
  modified_base 13: mod_base = cm
  modified_base 14: mod_base = OTHER; note = 2'-fluorouridine
  modified_base 15: mod_base = cm
  modified_base 16: mod_base = OTHER; note = 2'-fluorouridine
  modified_base 17: mod_base = um
  modified_base 18: mod_base = gm
  modified_base 19: mod_base = cm
  modified_base 20: mod_base = gm
  modified_base 21: mod_base = OTHER; note = 2'-O-methylguanosine with a 3'-phosphorothioate linkage
  modified_base 22: mod_base = OTHER; note = 2'-O-methylguanosine with a 3'-phosphorothioate linkage
  modified_base 23: mod_base = um
tccctaaagaacctcttgcgggt

SEQ ID NO: 40 - RNA, 21 nt, synthetic construct
  source 1..21: mol_type = other RNA
  modified_base 1: mod_base = OTHER; note = 2'-O-methylcytidine with a 3'-phosphorothioate linkage
  modified_base 2: mod_base = OTHER; note = 2'-O-methylcytidine with a 3'-phosphorothioate linkage
  modified_base 3: mod_base = gm
  modified_base 4: mod_base = cm
  modified_base 5: mod_base = OTHER; note = 2'-O-methyladenosine
  modified_base 6: mod_base = OTHER; note = 2'-O-methyladenosine
  modified_base 7: mod_base = gm
  modified_base 8: mod_base = OTHER; note = 2'-O-methyladenosine
  modified_base 9: mod_base = OTHER; note = 2'-fluoroguanosine
  modified_base 10: mod_base = OTHER; note = 2'-fluoroguanosine
  modified_base 11: mod_base = OTHER; note = 2'-fluorouridine
  modified_base 12: mod_base = um
  modified_base 13: mod_base = cm
  modified_base 14: mod_base = um
  modified_base 15: mod_base = um
  modified_base 16: mod_base = um
  modified_base 17: mod_base = OTHER; note = 2'-O-methyladenosine
  modified_base 18: mod_base = gm
  modified_base 19: mod_base = OTHER; note = 2'-O-methylguanosine with a 3'-phosphorothioate linkage
  modified_base 20: mod_base = OTHER; note = 2'-O-methylguanosine with a 3'-phosphorothioate linkage
  modified_base 21: mod_base = OTHER; note = 2'-O-methyladenosine
ccgcaagaggttctttaggga

SEQ ID NO: 41 - RNA, 23 nt, synthetic construct
  source 1..23: mol_type = other RNA
  modified_base 1: mod_base = OTHER; note = 2'-O-methyluridine with a 3'-phosphorothioate linkage
  modified_base 2: mod_base = OTHER; note = 2'-fluorocytidine with a 3'-phosphorothioate linkage
  modified_base 3: mod_base = cm
  modified_base 4: mod_base = cm
  modified_base 5: mod_base = OTHER; note = 2'-fluorouridine
  modified_base 6: mod_base = OTHER; note = 2'-O-methyladenosine
  modified_base 7: mod_base = OTHER; note = 2'-fluoroadenosine
  modified_base 8: mod_base = OTHER; note = 2'-O-methyladenosine
  modified_base 9: mod_base = gm
  modified_base 10: mod_base = OTHER; note = 2'-O-methyladenosine
  modified_base 11: mod_base = OTHER; note = 2'-O-methyladenosine
  modified_base 12: mod_base = cm
  modified_base 13: mod_base = cm
  modified_base 14: mod_base = OTHER; note = 2'-fluorouridine
  modified_base 15: mod_base = cm
  modified_base 16: mod_base = OTHER; note = 2'-fluorouridine
  modified_base 17: mod_base = um
  modified_base 18: mod_base = gm
  modified_base 19: mod_base = cm
  modified_base 20: mod_base = gm
  modified_base 21: mod_base = OTHER; note = 2'-O-methylguanosine with a 3'-phosphorothioate linkage
  modified_base 22: mod_base = OTHER; note = 2'-O-methylguanosine with a 3'-phosphorothioate linkage
  modified_base 23: mod_base = um
tccctaaagaacctcttgcgggt

SEQ ID NO: 42 - RNA, 23 nt, synthetic construct
  source 1..23: mol_type = other RNA
  modified_base 1: mod_base = OTHER; note = 2'-O-methyluridine with a 3'-phosphorothioate linkage
  modified_base 2: mod_base = OTHER; note = 2'-fluorocytidine with a 3'-phosphorothioate linkage
  modified_base 3: mod_base = OTHER; note = 2'-fluorocytidine
  modified_base 4: mod_base = cm
  modified_base 5: mod_base = um
  modified_base 6: mod_base = OTHER; note = 2'-O-methyladenosine
  modified_base 7: mod_base = OTHER; note = 2'-fluoroadenosine
  modified_base 8: mod_base = OTHER; note = 2'-O-methyladenosine
  modified_base 9: mod_base = gm
  modified_base 10: mod_base = OTHER; note = 2'-O-methyladenosine
  modified_base 11: mod_base = OTHER; note = 2'-O-methyladenosine
  modified_base 12: mod_base = cm
  modified_base 13: mod_base = cm
  modified_base 14: mod_base = OTHER; note = 2'-fluorouridine
  modified_base 15: mod_base = cm
  modified_base 16: mod_base = OTHER; note = 2'-fluorouridine
  modified_base 17: mod_base = um
  modified_base 18: mod_base = gm
  modified_base 19: mod_base = cm
  modified_base 20: mod_base = gm
  modified_base 21: mod_base = OTHER; note = 2'-O-methylguanosine with a 3'-phosphorothioate linkage
  modified_base 22: mod_base = OTHER; note = 2'-O-methylguanosine with a 3'-phosphorothioate linkage
  modified_base 23: mod_base = um
tccctaaagaacctcttgcgggt

SEQ ID NO: 43 - RNA, 23 nt, synthetic construct
  source 1..23: mol_type = other RNA
  modified_base 1: mod_base = OTHER; note = 2'-O-methyluridine with a 3'-phosphorothioate linkage
  modified_base 2: mod_base = OTHER; note = 2'-fluorocytidine with a 3'-phosphorothioate linkage
  modified_base 3: mod_base = cm
  modified_base 4: mod_base = cm
  modified_base 5: mod_base = OTHER; note = 2'-fluorouridine
  modified_base 6: mod_base = OTHER; note = 2'-O-methyladenosine
  modified_base 7: mod_base = OTHER; note = 2'-O-methyladenosine
  modified_base 8: mod_base = OTHER; note = 2'-fluoroadenosine
  modified_base 9: mod_base = gm
  modified_base 10: mod_base = OTHER; note = 2'-O-methyladenosine
  modified_base 11: mod_base = OTHER; note = 2'-O-methyladenosine
  modified_base 12: mod_base = cm
  modified_base 13: mod_base = cm
  modified_base 14: mod_base = OTHER; note = 2'-fluorouridine
  modified_base 15: mod_base = cm
  modified_base 16: mod_base = OTHER; note = 2'-fluorouridine
  modified_base 17: mod_base = um
  modified_base 18: mod_base = gm
  modified_base 19: mod_base = cm
  modified_base 20: mod_base = gm
  modified_base 21: mod_base = OTHER; note = 2'-O-methylguanosine with a 3'-phosphorothioate linkage
  modified_base 22: mod_base = OTHER; note = 2'-O-methylguanosine with a 3'-phosphorothioate linkage
  modified_base 23: mod_base = um
tccctaaagaacctcttgcgggt

SEQ ID NO: 44 - RNA, 23 nt, synthetic construct
  source 1..23: mol_type = other RNA
  modified_base 1: mod_base = OTHER; note = 2'-O-methyluridine with a 3'-phosphorothioate linkage
  modified_base 2: mod_base = OTHER; note = 2'-fluorocytidine with a 3'-phosphorothioate linkage
  modified_base 3: mod_base = cm
  modified_base 4: mod_base = cm
  modified_base 5: mod_base = um
  modified_base 6: mod_base = OTHER; note = 2'-O-methyladenosine
  modified_base 7: mod_base = OTHER; note = 2'-O-methyladenosine
  modified_base 8: mod_base = OTHER; note = 2'-O-methyladenosine
  modified_base 9: mod_base = gm
  modified_base 10: mod_base = OTHER; note = 2'-O-methyladenosine
  modified_base 11: mod_base = OTHER; note = 2'-O-methyladenosine
  modified_base 12: mod_base = cm
  modified_base 13: mod_base = cm
  modified_base 14: mod_base = OTHER; note = 2'-fluorouridine
  modified_base 15: mod_base = cm
  modified_base 16: mod_base = um
  modified_base 17: mod_base = um
  modified_base 18: mod_base = gm
  modified_base 19: mod_base = cm
  modified_base 20: mod_base = gm
  modified_base 21: mod_base = OTHER; note = 2'-O-methylguanosine with a 3'-phosphorothioate linkage
  modified_base 22: mod_base = OTHER; note = 2'-O-methylguanosine with a 3'-phosphorothioate linkage
  modified_base 23: mod_base = um
tccctaaagaacctcttgcgggt

SEQ ID NO: 45 - RNA, 23 nt, synthetic construct
  source 1..23: mol_type = other RNA
  modified_base 1: mod_base = OTHER; note = 2'-O-methyluridine with a 3'-phosphorothioate linkage
  modified_base 2: mod_base = OTHER; note = 2'-fluoroguanosine with a 3'-phosphorothioate linkage
  modified_base 3: mod_base = um
  modified_base 4: mod_base = OTHER; note = 2'-O-methyladenosine
  modified_base 5: mod_base = gm
  modified_base 6: mod_base = OTHER; note = 2'-fluoroadenosine
  modified_base 7: mod_base = OTHER; note = 2'-O-methyladenosine
  modified_base 8: mod_base = um
  modified_base 9: mod_base = OTHER; note = 2'-O-methyladenosine
  modified_base 10: mod_base = gm
  modified_base 11: mod_base = um
  modified_base 12: mod_base = um
  modified_base 13: mod_base = gm
  modified_base 14: mod_base = OTHER; note = 2'-fluoroguanosine
  modified_base 15: mod_base = cm
  modified_base 16: mod_base = OTHER; note = 2'-fluoroguanosine
  modified_base 17: mod_base = OTHER; note = 2'-O-methyladenosine
  modified_base 18: mod_base = OTHER; note = 2'-O-methyladenosine
  modified_base 19: mod_base = gm
  modified_base 20: mod_base = gm
  modified_base 21: mod_base = OTHER; note = 2'-O-methyluridine with a 3'-phosphorothioate linkage
  modified_base 22: mod_base = OTHER; note = 2'-O-methylcytidine with a 3'-phosphorothioate linkage
  modified_base 23: mod_base = um
tgtagaatagttggcgaaggtct

SEQ ID NO: 46 - RNA, 23 nt, synthetic construct
  source 1..23: mol_type = other RNA
  modified_base 1: mod_base = OTHER; note = 2'-O-methyluridine with a 3'-phosphorothioate linkage
  modified_base 2: mod_base = OTHER; note = 2'-fluorocytidine with a 3'-phosphorothioate linkage
  modified_base 3: mod_base = gm
  modified_base 4: mod_base = cm
  modified_base 5: mod_base = OTHER; note = 2'-O-methyladenosine
  modified_base 6: mod_base = OTHER; note = 2'-fluorocytidine
  modified_base 7: mod_base = gm
  modified_base 8: mod_base = um
  modified_base 9: mod_base = OTHER; note = 2'-O-methyladenosine
  modified_base 10: mod_base = gm
  modified_base 11: mod_base = OTHER; note = 2'-O-methyladenosine
  modified_base 12: mod_base = OTHER; note = 2'-O-methyladenosine
  modified_base 13: mod_base = um
  modified_base 14: mod_base = OTHER; note = 2'-fluoroadenosine
  modified_base 15: mod_base = gm
  modified_base 16: mod_base = OTHER; note = 2'-fluorouridine
  modified_base 17: mod_base = um
  modified_base 18: mod_base = gm
  modified_base 19: mod_base = gm
  modified_base 20: mod_base = cm
  modified_base 21: mod_base = OTHER; note = 2'-O-methylguanosine with a 3'-phosphorothioate linkage
  modified_base 22: mod_base = OTHER; note = 2'-O-methyladenosine with a 3'-phosphorothioate linkage
  modified_base 23: mod_base = OTHER; note = 2'-O-methyladenosine
tcgcacgtagaatagttggcgaa

SEQ ID NO: 47 - RNA, 23 nt, synthetic construct
  source 1..23: mol_type = other RNA
  modified_base 1: mod_base = OTHER; note = 2'-O-methyluridine with a 3'-phosphorothioate linkage
  modified_base 2: mod_base = OTHER; note = 2'-fluoroguanosine with a 3'-phosphorothioate linkage
  modified_base 3: mod_base = OTHER; note = 2'-O-methyladenosine
  modified_base 4: mod_base = gm
  modified_base 5: mod_base = um
  modified_base 6: mod_base = OTHER; note = 2'-fluoroadenosine
  modified_base 7: mod_base = gm
  modified_base 8: mod_base = gm
  modified_base 9: mod_base = OTHER; note = 2'-O-methyladenosine
  modified_base 10: mod_base = OTHER; note = 2'-O-methyladenosine
  modified_base 11: mod_base = OTHER; note = 2'-O-methyladenosine
  modified_base 12: mod_base = cm
  modified_base 13: mod_base = cm
  modified_base 14: mod_base = OTHER; note = 2'-fluoroadenosine
  modified_base 15: mod_base = gm
  modified_base 16: mod_base = OTHER; note = 2'-fluoroguanosine
  modified_base 17: mod_base = cm
  modified_base 18: mod_base = gm
  modified_base 19: mod_base = um
  modified_base 20: mod_base = um
  modified_base 21: mod_base = OTHER; note = 2'-O-methyluridine with a 3'-phosphorothioate linkage
  modified_base 22: mod_base = OTHER; note = 2'-O-methylcytidine with a 3'-phosphorothioate linkage
  modified_base 23: mod_base = OTHER; note = 2'-O-methyladenosine
tgagtaggaaaccaggcgtttca

(19) United States
(12) Patent Application Publication
(10) Pub. No.: US 2025/0188472 A1
(43) Pub. Date: Jun. 12, 2025

(54) SARM1 RNA INTERFERENCE AGENTS

(71) Applicant: ELI LILLY AND COMPANY, Indianapolis, IN (US)

(72) Inventors: Nicholas Alan BABB, Noblesville, IN (US); Selene HERNANDEZ BUQUER, Indianapolis, IN (US); Yea Jin KAESER-WOO, Newton, MA (US); Gregory Lawrence LACKNER, Indianapolis, IN (US); Rebecca Ruth MILES, Pendleton, IN (US); Douglas Raymond PERKINS, New Palestine, IN (US); Jibo WANG, Carmel, IN (US); Yaming WANG, Zionsville, IN (US)

(21) Appl. No.: 18/846,293

(22) PCT Filed: Feb. 28, 2023
(86) PCT No.: PCT/US2023/063385

§ 371 Date: Sep. 12, 2024

Related U.S. Application Data
(60) Provisional application No. 63/319,459, filed on Mar. 14, 2022.

(51) Int. Cl. C12N 15/113; A61P 25/00

(52) U.S. Cl. CPC C12N 15/1137; A61P 25/00; C12N 2310/11; C12N 2310/14; C12N 2310/315; C12N 2310/321; C12N 2310/322; C12Y 302/02006

(57) ABSTRACT

Provided herein are SARM1 RNAi agents and compositions comprising a SARM1 RNAi agent. Also provided herein are methods of using the SARM1 RNAi agents or compositions comprising a SARM1 RNAi agent for reducing SARM1 expression, reducing axon degeneration, and/or treating SARM1-mediated neurological disease in a subject.

### SEQUENCE LISTING

The present application is being filed along with a Sequence Listing in ST.26 XML format. The Sequence Listing is provided as a file titled “30243” created 13 Jan. 2023 and is 361 kilobytes in size. The Sequence Listing information in the ST.26 XML format is incorporated herein by reference in its entirety.

### BACKGROUND

Axon degeneration is a pathological feature of many neurological diseases including peripheral neuropathies, traumatic brain injury, and neurodegenerative diseases (Gerdts et al., Science, 2015, 348:453-457). Damaged or unhealthy axons are eliminated by an intrinsic self-destruction program known as Wallerian degeneration, which is an active process of retrograde degeneration of the distal end of an axon while keeping the proximal axon segment and cell body intact (Gerdts, J., et al., Neuron, 2016, 89, 449-460; Whitmore, A. et al., Cell Death Differ., 2003, 10, 260-261).

SARM1 (sterile alpha and TIR motif containing 1) is a NAD+ hydrolase that plays an important role in axon degeneration. It has been reported that knocking-down or eliminating the expression of SARM1 leads to long-lasting protection of sensory neurons against injury-induced axonal degeneration (Gerdts et al., J Neurosci, 2013, 33, 13569-13580). WO2021/108602 describes SARM1 antisense oligonucleotides.

RNA interference (RNAi) is a highly conserved regulatory mechanism in which RNA molecules are involved in sequence-specific suppression of gene expression by double-stranded RNA molecules (dsRNA) (Fire et al., Nature 391:806-811, 1998).

There remains a need for therapeutic agents that can inhibit or adjust the expression of SARM1 for treating SARM1-mediated neurological diseases e.g., by utilizing RNAi.

### SUMMARY OF INVENTION

Provided herein are SARM1 RNAi agents and compositions comprising a SARM1 RNAi agent. Also provided herein are methods of using the SARM1 RNAi agents or compositions comprising a SARM1 RNAi agent for reducing SARM1 expression, reducing axon degeneration, and/or treating SARM1-mediated neurological diseases in a subject.

In one aspect, provided herein are SARM1 RNAi agents having a sense strand and an antisense strand, wherein the sense strand and the antisense strand form a duplex, wherein the sense strand and the antisense strand comprise a pair of nucleic acid sequences selected from the group consisting of:
(a) the sense strand comprises a first nucleic acid sequence having at least 95% sequence identity to SEQ ID NO: 1, and the antisense strand comprises a second nucleic acid sequence having at least 95% sequence identity to SEQ ID NO: 2;
(b) the sense strand comprises a first nucleic acid sequence having at least 95% sequence identity to SEQ ID NO: 3, and the antisense strand comprises a second nucleic acid sequence having at least 95% sequence identity to SEQ ID NO: 4;
(c) the sense strand comprises a first nucleic acid sequence having at least 95% sequence identity to SEQ ID NO: 5, and the antisense strand comprises a second nucleic acid sequence having at least 95% sequence identity to SEQ ID NO: 6;
(d) the sense strand comprises a first nucleic acid sequence having at least 95% sequence identity to SEQ ID NO: 7, and the antisense strand comprises a second nucleic acid sequence having at least 95% sequence identity to SEQ ID NO: 8;
(e) the sense strand comprises a first nucleic acid sequence having at least 95% sequence identity to SEQ ID NO: 9, and the antisense strand comprises a second nucleic acid sequence having at least 95% sequence identity to SEQ ID NO: 10; and
(f) the sense strand comprises a first nucleic acid sequence having at least 95% sequence identity to SEQ ID NO: 11, and the antisense strand comprises a second nucleic acid sequence having at least 95% sequence identity to SEQ ID NO: 12,
wherein optionally one or more nucleotides of the sense strand and the antisense strand are independently modified nucleotides, and wherein optionally one or more internucleotide linkages of the sense strand and the antisense strand are modified internucleotide linkages.

In some embodiments, the sense strand and the antisense strand of the SARM1 RNAi agent described herein comprise a pair of nucleic acid sequences selected from the group consisting of:
(a) the sense strand comprises a first nucleic acid sequence of SEQ ID NO: 1, and the antisense strand comprises a second nucleic acid sequence of SEQ ID NO: 2;
(b) the sense strand comprises a first nucleic acid sequence of SEQ ID NO: 3, and the antisense strand comprises a second nucleic acid sequence of SEQ ID NO: 4;
(c) the sense strand comprises a first nucleic acid sequence of SEQ ID NO: 5, and the antisense strand comprises a second nucleic acid sequence of SEQ ID NO: 6;
(d) the sense strand comprises a first nucleic acid sequence of SEQ ID NO: 7, and the antisense strand comprises a second nucleic acid sequence of SEQ ID NO: 8;
(e) the sense strand comprises a first nucleic acid sequence of SEQ ID NO: 9, and the antisense strand comprises a second nucleic acid sequence of SEQ ID NO: 10; and
(f) the sense strand comprises a first nucleic acid sequence of SEQ ID NO: 11, and the antisense strand comprises a second nucleic acid sequence of SEQ ID NO: 12,
wherein optionally one or more nucleotides of the sense strand and the antisense strand are independently modified nucleotides, and wherein optionally one or more internucleotide linkages of the sense strand and the antisense strand are modified internucleotide linkages.

In some embodiments, the sense strand and the antisense strand of the SARM1 RNAi agent described herein have a pair of nucleic acid sequences selected from the group consisting of:
(a) the sense strand has (e.g., consists of, or consists essentially of) a first nucleic acid sequence of SEQ ID NO: 1, and the antisense strand has (e.g., consists of, or consists essentially of) a second nucleic acid sequence of SEQ ID NO: 2;
(b) the sense strand has (e.g., consists of, or consists essentially of) a first nucleic acid sequence of SEQ ID NO: 3, and the antisense strand has (e.g., consists of, or consists essentially of) a second nucleic acid sequence of SEQ ID NO: 4;
(c) the sense strand has (e.g., consists of, or consists essentially of) a first nucleic acid sequence of SEQ ID NO: 5, and the antisense strand has (e.g., consists of, or consists essentially of) a second nucleic acid sequence of SEQ ID NO: 6;
(d) the sense strand has (e.g., consists of, or consists essentially of) a first nucleic acid sequence of SEQ ID NO: 7, and the antisense strand has (e.g., consists of, or consists essentially of) a second nucleic acid sequence of SEQ ID NO: 8;
(e) the sense strand has (e.g., consists of, or consists essentially of) a first nucleic acid sequence of SEQ ID NO: 9, and the antisense strand has (e.g., consists of, or consists essentially of) a second nucleic acid sequence of SEQ ID NO: 10; and
(f) the sense strand has (e.g., consists of, or consists essentially of) a first nucleic acid sequence of SEQ ID NO: 11, and the antisense strand has (e.g., consists of, or consists essentially of) a second nucleic acid sequence of SEQ ID NO: 12.

The SARM1 RNAi agents described herein may include modifications. The modifications can be made to one or more nucleotides of the sense strand and/or antisense strand or to the internucleotide linkages. In some embodiments, one or more nucleotides of the sense strand are modified nucleotides. In some embodiments, each nucleotide of the sense strand is a modified nucleotide. In some embodiments, one or more nucleotides of the antisense strand are modified nucleotides. In some embodiments, each nucleotide of the antisense strand is a modified nucleotide. In some embodiments, the modified nucleotide is a 2′-fluoro modified nucleotide, 2′-O-methyl modified nucleotide, or 2′-O-alkyl modified nucleotide, e.g., 2′-O—C12-16 alkyl modified nucleotide. In some embodiments, the sense strand has four 2′-fluoro modified nucleotides at positions 7, 9, 10, and 11 from the 5′ end of the sense strand. In some embodiments, nucleotides at positions other than positions 7, 9, 10, and 11 of the sense strand are 2′-O-methyl modified nucleotides. In some embodiments, the antisense strand has four 2′-fluoro modified nucleotides at positions 2, 6, 14, and 16 from the 5′ end of the antisense strand. In some embodiments, nucleotides at positions other than positions 2, 6, 14 and 16 of the antisense strand are 2′-O-methyl modified nucleotides. In some embodiments, the sense strand has three 2′-fluoro modified nucleotides at positions 9, 10, and 11 from the 5′ end of the sense strand. In some embodiments, the nucleotides at positions other than positions 9, 10, and 11 of the sense strand are 2′-O-methyl modified nucleotides. In some embodiments, the antisense strand has five 2′-fluoro modified nucleotides at positions 2, 5, 7, 14, and 16 from the 5′ end of the antisense strand. In some embodiments, the nucleotides at positions other than positions 2, 5, 7, 14, and 16 of the antisense strand are 2′-O-methyl modified nucleotides. In some embodiments, the antisense strand has five 2′-fluoro modified nucleotides at positions 2, 5, 8, 14, and 16 from the 5′ end of the antisense strand. In some embodiments, the nucleotides at positions other than positions 2, 5, 8, 14, and 16 of the antisense strand are 2′-O-methyl modified nucleotides. In some embodiments, the antisense strand has five 2′-fluoro modified nucleotides at positions 2, 3, 7, 14, and 16 from the 5′ end of the antisense strand. In some embodiments, the nucleotides at positions other than positions 2, 3, 7, 14, and 16 of the antisense strand are 2′-O-methyl modified nucleotides. In some embodiments, the antisense strand has two 2′-fluoro modified nucleotides at positions 2 and 14 from the 5′ end of the antisense strand. In some embodiments, the nucleotides at positions other than positions 2 and 14 of the antisense strand are 2′-O-methyl modified nucleotides.

In some embodiments, the first nucleotide from the 5′ end of the antisense strand is a modified nucleotide that has a phosphate analog, e.g., a 5′-vinylphosphonate. In some embodiments, the first nucleotide from the 5′ end of the antisense strand has a 5′ phosphate. In some embodiments, the sense strand comprises an abasic moiety or inverted abasic moiety.

In some embodiments, the sense strand and the antisense strand have one or more modified internucleotide linkages, e.g., phosphorothioate linkage. In some embodiments, the sense strand has four or five phosphorothioate linkages. In some embodiments, the antisense strand has four or five phosphorothioate linkages. In some embodiments, the sense strand has four phosphorothioate linkages and the antisense strand has four phosphorothioate linkages.

In some embodiments, provided herein are SARM1 RNAi agents having a sense strand and an antisense strand, wherein the sense strand and the antisense strand form a duplex, wherein the sense strand and the antisense strand comprise a pair of nucleic acid sequences selected from the group consisting of:
(a) the sense strand comprises a first nucleic acid sequence of SEQ ID NO: 13, and the antisense strand comprises a second nucleic acid sequence of SEQ ID NO: 14;
(b) the sense strand comprises a first nucleic acid sequence of SEQ ID NO: 15, and the antisense strand comprises a second nucleic acid sequence of SEQ ID NO: 16;
(c) the sense strand comprises a first nucleic acid sequence of SEQ ID NO: 17, and the antisense strand comprises a second nucleic acid sequence of SEQ ID NO: 18;
(d) the sense strand comprises a first nucleic acid sequence of SEQ ID NO: 19, and the antisense strand comprises a second nucleic acid sequence of SEQ ID NO: 20;
(e) the sense strand comprises a first nucleic acid sequence of SEQ ID NO: 21, and the antisense strand comprises a second nucleic acid sequence of SEQ ID NO: 22;
(f) the sense strand comprises a first nucleic acid sequence of SEQ ID NO: 23, and the antisense strand comprises a second nucleic acid sequence of SEQ ID NO: 24;
(g) the sense strand comprises a first nucleic acid sequence of SEQ ID NO: 15, and the antisense strand comprises a second nucleic acid sequence of SEQ ID NO: 27;
(h) the sense strand comprises a first nucleic acid sequence of SEQ ID NO: 28, and the antisense strand comprises a second nucleic acid sequence of SEQ ID NO: 29;
(i) the sense strand comprises a first nucleic acid sequence of SEQ ID NO: 28, and the antisense strand comprises a second nucleic acid sequence of SEQ ID NO: 30;
(j) the sense strand comprises a first nucleic acid sequence of SEQ ID NO: 28, and the antisense strand comprises a second nucleic acid sequence of SEQ ID NO: 31;
(k) the sense strand comprises a first nucleic acid sequence of SEQ ID NO: 28, and the antisense strand comprises a second nucleic acid sequence of SEQ ID NO: 32;
(l) the sense strand comprises a first nucleic acid sequence of SEQ ID NO: 13, and the antisense strand comprises a second nucleic acid sequence of SEQ ID NO: 33;
(m) the sense strand comprises a first nucleic acid sequence of SEQ ID NO: 34, and the antisense strand comprises a second nucleic acid sequence of SEQ ID NO: 35;
(n) the sense strand comprises a first nucleic acid sequence of SEQ ID NO: 34, and the antisense strand comprises a second nucleic acid sequence of SEQ ID NO: 36;
(o) the sense strand comprises a first nucleic acid sequence of SEQ ID NO: 34, and the antisense strand comprises a second nucleic acid sequence of SEQ ID NO: 37;
(p) the sense strand comprises a first nucleic acid sequence of SEQ ID NO: 34, and the antisense strand comprises a second nucleic acid sequence of SEQ ID NO: 38;
(q) the sense strand comprises a first nucleic acid sequence of SEQ ID NO: 21, and the antisense strand comprises a second nucleic acid sequence of SEQ ID NO: 39;
(r) the sense strand comprises a first nucleic acid sequence of SEQ ID NO: 40, and the antisense strand comprises a second nucleic acid sequence of SEQ ID NO: 41;
(s) the sense strand comprises a first nucleic acid sequence of SEQ ID NO: 40, and the antisense strand comprises a second nucleic acid sequence of SEQ ID NO: 42;
(t) the sense strand comprises a first nucleic acid sequence of SEQ ID NO: 40, and the antisense strand comprises a second nucleic acid sequence of SEQ ID NO: 43;
(u) the sense strand comprises a first nucleic acid sequence of SEQ ID NO: 40, and the antisense strand comprises a second nucleic acid sequence of SEQ ID NO: 44;
(v) the sense strand comprises a first nucleic acid sequence of SEQ ID NO: 17, and the antisense strand comprises a second nucleic acid sequence of SEQ ID NO: 45;
(w) the sense strand comprises a first nucleic acid sequence of SEQ ID NO: 19, and the antisense strand comprises a second nucleic acid sequence of SEQ ID NO: 46; and
(x) the sense strand comprises a first nucleic acid sequence of SEQ ID NO: 23, and the antisense strand comprises a second nucleic acid sequence of SEQ ID NO: 47.

In some embodiments, provided herein are SARM1 RNAi agents having a sense strand and an antisense strand, wherein the sense strand and the antisense strand form a duplex, wherein the sense strand and the antisense strand have a pair of nucleic acid sequences selected from the group consisting of:
(a) the sense strand has (e.g., consists of, or consists essentially of) a first nucleic acid sequence of SEQ ID NO: 13, and the antisense strand has (e.g., consists of, or consists essentially of) a second nucleic acid sequence of SEQ ID NO: 14;
(b) the sense strand has (e.g., consists of, or consists essentially of) a first nucleic acid sequence of SEQ ID NO: 15, and the antisense strand has (e.g., consists of, or consists essentially of) a second nucleic acid sequence of SEQ ID NO: 16;
(c) the sense strand has (e.g., consists of, or consists essentially of) a first nucleic acid sequence of SEQ ID NO: 17, and the antisense strand has (e.g., consists of, or consists essentially of) a second nucleic acid sequence of SEQ ID NO: 18;
(d) the sense strand has (e.g., consists of, or consists essentially of) a first nucleic acid sequence of SEQ ID NO: 19, and the antisense strand has (e.g., consists of, or consists essentially of) a second nucleic acid sequence of SEQ ID NO: 20;
(e) the sense strand has (e.g., consists of, or consists essentially of) a first nucleic acid sequence of SEQ ID NO: 21, and the antisense strand has (e.g., consists of, or consists essentially of) a second nucleic acid sequence of SEQ ID NO: 22;
(f) the sense strand has (e.g., consists of, or consists essentially of) a first nucleic acid sequence of SEQ ID NO: 23, and the antisense strand has (e.g., consists of, or consists essentially of) a second nucleic acid sequence of SEQ ID NO: 24;
(g) the sense strand has (e.g., consists of, or consists essentially of) a first nucleic acid sequence of SEQ ID NO: 15, and the antisense strand has (e.g., consists of, or consists essentially of) a second nucleic acid sequence of SEQ ID NO: 27;
(h) the sense strand has (e.g., consists of, or consists essentially of) a first nucleic acid sequence of SEQ ID NO: 28, and the antisense strand has (e.g., consists of, or consists essentially of) a second nucleic acid sequence of SEQ ID NO: 29;
(i) the sense strand has (e.g., consists of, or consists essentially of) a first nucleic acid sequence of SEQ ID NO: 28, and the antisense strand has (e.g., consists of, or consists essentially of) a second nucleic acid sequence of SEQ ID NO: 30;
(j) the sense strand has (e.g., consists of, or consists essentially of) a first nucleic acid sequence of SEQ ID NO: 28, and the antisense strand has (e.g., consists of, or consists essentially of) a second nucleic acid sequence of SEQ ID NO: 31;
(k) the sense strand has (e.g., consists of, or consists essentially of) a first nucleic acid sequence of SEQ ID NO: 28, and the antisense strand has (e.g., consists of, or consists essentially of) a second nucleic acid sequence of SEQ ID NO: 32;
(l) the sense strand has (e.g., consists of, or consists essentially of) a first nucleic acid sequence of SEQ ID NO: 13, and the antisense strand has (e.g., consists of, or consists essentially of) a second nucleic acid sequence of SEQ ID NO: 33;
(m) the sense strand has (e.g., consists of, or consists essentially of) a first nucleic acid sequence of SEQ ID NO: 34, and the antisense strand has (e.g., consists of, or consists essentially of) a second nucleic acid sequence of SEQ ID NO: 35;
(n) the sense strand has (e.g., consists of, or consists essentially of) a first nucleic acid sequence of SEQ ID NO: 34, and the antisense strand has (e.g., consists of, or consists essentially of) a second nucleic acid sequence of SEQ ID NO: 36;
(o) the sense strand has (e.g., consists of, or consists essentially of) a first nucleic acid sequence of SEQ ID NO: 34, and the antisense strand has (e.g., consists of, or consists essentially of) a second nucleic acid sequence of SEQ ID NO: 37;
(p) the sense strand has (e.g., consists of, or consists essentially of) a first nucleic acid sequence of SEQ ID NO: 34, and the antisense strand has (e.g., consists of, or consists essentially of) a second nucleic acid sequence of SEQ ID NO: 38;
(q) the sense strand has (e.g., consists of, or consists essentially of) a first nucleic acid sequence of SEQ ID NO: 21, and the antisense strand has (e.g., consists of, or consists essentially of) a second nucleic acid sequence of SEQ ID NO: 39;
(r) the sense strand has (e.g., consists of, or consists essentially of) a first nucleic acid sequence of SEQ ID NO: 40, and the antisense strand has (e.g., consists of, or consists essentially of) a second nucleic acid sequence of SEQ ID NO: 41;
(s) the sense strand has (e.g., consists of, or consists essentially of) a first nucleic acid sequence of SEQ ID NO: 40, and the antisense strand has (e.g., consists of, or consists essentially of) a second nucleic acid sequence of SEQ ID NO: 42;
(t) the sense strand has (e.g., consists of, or consists essentially of) a first nucleic acid sequence of SEQ ID NO: 40, and the antisense strand has (e.g., consists of, or consists essentially of) a second nucleic acid sequence of SEQ ID NO: 43;
(u) the sense strand has (e.g., consists of, or consists essentially of) a first nucleic acid sequence of SEQ ID NO: 40, and the antisense strand has (e.g., consists of, or consists essentially of) a second nucleic acid sequence of SEQ ID NO: 44;
(v) the sense strand has (e.g., consists of, or consists essentially of) a first nucleic acid sequence of SEQ ID NO: 17, and the antisense strand has (e.g., consists of, or consists essentially of) a second nucleic acid sequence of SEQ ID NO: 45;
(w) the sense strand has (e.g., consists of, or consists essentially of) a first nucleic acid sequence of SEQ ID NO: 19, and the antisense strand has (e.g., consists of, or consists essentially of) a second nucleic acid sequence of SEQ ID NO: 46; and
(x) the sense strand has (e.g., consists of, or consists essentially of) a first nucleic acid sequence of SEQ ID NO: 23, and the antisense strand has (e.g., consists of, or consists essentially of) a second nucleic acid sequence of SEQ ID NO: 47.

In some embodiments, the sense strand of the SARM1 RNAi agent has a delivery moiety. In some embodiments, the sense strand of the SARM1 RNAi agent has a delivery moiety conjugated to the 3′ end of the sense strand. In some embodiments, the sense strand of the SARM1 RNAi agent has a delivery moiety conjugated to the 5′ end of the sense strand. In some embodiments, the sense strand of the SARM1 RNAi agent has delivery moieties conjugated to both the 5′ end and the 3′ end of the sense strand. In some embodiments, the sense strand of the SARM1 RNAi agent has a delivery moiety conjugated to a nucleotide of the sense strand. In some embodiments, the delivery moiety is a-tocopherol, palmitic acid, or another moiety in Table 4. In some embodiments, the delivery moiety is conjugated to the 5′ or 3′ end of the sense stand via a linker, e.g., a linker of Table 5. In some embodiments, the SARM1 RNAi agent is not a lipid conjugate. In some embodiments, the SARM1 RNAi agent has no delivery moiety.

In a further aspect, provided herein are SARM1 RNAi agents of Formula (I): R-L-D, wherein R is a double stranded RNA (dsRNA) having a sense stand and an antisense strand, wherein the sense strand and the antisense strand form a duplex; wherein D is a delivery means (e.g., delivery moiety) for delivering the dsRNA into a cell; and wherein L is a linking means (e.g., linker) for linking the dsRNA to the delivery means, or optionally absent, wherein the sense strand and the antisense strand comprises a pair of nucleic acid sequences selected from the group consisting of:
(a) the sense strand comprises a first nucleic acid sequence of SEQ ID NO: 1, and the antisense strand comprises a second nucleic acid sequence of SEQ ID NO: 2;
(b) the sense strand comprises a first nucleic acid sequence of SEQ ID NO: 3, and the antisense strand comprises a second nucleic acid sequence of SEQ ID NO: 4;
(c) the sense strand comprises a first nucleic acid sequence of SEQ ID NO: 5, and the antisense strand comprises a second nucleic acid sequence of SEQ ID NO: 6;
(d) the sense strand comprises a first nucleic acid sequence of SEQ ID NO: 7, and the antisense strand comprises a second nucleic acid sequence of SEQ ID NO: 8;
(e) the sense strand comprises a first nucleic acid sequence of SEQ ID NO: 9, and the antisense strand comprises a second nucleic acid sequence of SEQ ID NO: 10; and
(f) the sense strand comprises a first nucleic acid sequence of SEQ ID NO: 11, and the antisense strand comprises a second nucleic acid sequence of SEQ ID NO: 12,
wherein optionally one or more nucleotides of the sense strand and the antisense strand are independently modified nucleotides, and wherein optionally one or more internucleotide linkages of the sense strand and the antisense strand are modified internucleotide linkages.

In some embodiments, provided herein are SARM1 RNAi agents of Formula (I): R-L-D, wherein R is a dsRNA having a sense stand and an antisense strand, wherein the sense strand and the antisense strand form a duplex; wherein D is a delivery means (e.g., a delivery moiety) for delivering the dsRNA into a cell; and wherein L is a linking means (e.g., a linker) for linking the dsRNA to the delivery means, or optionally absent, wherein the sense strand and the antisense strand have a pair of nucleic acid sequences selected from the group consisting of:
(a) the sense strand has (e.g., consists of, or consists essentially of) a first nucleic acid sequence of SEQ ID NO: 1, and the antisense strand has (e.g., consist of, or consists essentially of) a second nucleic acid sequence of SEQ ID NO: 2;
(b) the sense strand has (e.g., consists of, or consists essentially of) a first nucleic acid sequence of SEQ ID NO: 3, and the antisense strand has (e.g., consist of, or consists essentially of) a second nucleic acid sequence of SEQ ID NO: 4;
(c) the sense strand has (e.g., consists of, or consists essentially of) a first nucleic acid sequence of SEQ ID NO: 5, and the antisense strand has (e.g., consists of, or consists essentially of) a second nucleic acid sequence of SEQ ID NO: 6;
(d) the sense strand has (e.g., consists of, or consists essentially of) a first nucleic acid sequence of SEQ ID NO: 7, and the antisense strand has (e.g., consists of, or consists essentially of) a second nucleic acid sequence of SEQ ID NO: 8;
(e) the sense strand has (e.g., consists of, or consists essentially of) a first nucleic acid sequence of SEQ ID NO: 9, and the antisense strand has (e.g., consists of, or consists essentially of) a second nucleic acid sequence of SEQ ID NO: 10; and
(f) the sense strand has (e.g., consists of, or consists essentially of) a first nucleic acid sequence of SEQ ID NO: 11, and the antisense strand has (e.g., consists of, or consists essentially of) a second nucleic acid sequence of SEQ ID NO: 12,
wherein optionally one or more nucleotides of the sense strand and the antisense strand are independently modified nucleotides, and wherein optionally one or more internucleotide linkages of the sense strand and the antisense strand are modified internucleotide linkages.

In some embodiments, provided herein are SARM1 RNAi agents of Formula (I): R-L-D, wherein R is a dsRNA having a sense stand and an antisense strand, wherein the sense strand and the antisense strand form a duplex; wherein D is a delivery means (e.g., delivery moiety) for delivering the dsRNA into a cell; and wherein L is a linking means (e.g., linker) for linking the dsRNA to the delivery means, or optionally absent, wherein the sense strand and the antisense strand comprise a pair of nucleic acid sequences selected from the group consisting of:
(a) the sense strand comprises a first nucleic acid sequence of SEQ ID NO: 13, and the antisense strand comprises a second nucleic acid sequence of SEQ ID NO: 14;
(b) the sense strand comprises a first nucleic acid sequence of SEQ ID NO: 15, and the antisense strand comprises a second nucleic acid sequence of SEQ ID NO: 16;
(c) the sense strand comprises a first nucleic acid sequence of SEQ ID NO: 17, and the antisense strand comprises a second nucleic acid sequence of SEQ ID NO: 18;
(d) the sense strand comprises a first nucleic acid sequence of SEQ ID NO: 19, and the antisense strand comprises a second nucleic acid sequence of SEQ ID NO: 20;
(e) the sense strand comprises a first nucleic acid sequence of SEQ ID NO: 21, and the antisense strand comprises a second nucleic acid sequence of SEQ ID NO: 22;
(f) the sense strand comprises a first nucleic acid sequence of SEQ ID NO: 23, and the antisense strand comprises a second nucleic acid sequence of SEQ ID NO: 24;
(g) the sense strand comprises a first nucleic acid sequence of SEQ ID NO: 15, and the antisense strand comprises a second nucleic acid sequence of SEQ ID NO: 27;
(h) the sense strand comprises a first nucleic acid sequence of SEQ ID NO: 28, and the antisense strand comprises a second nucleic acid sequence of SEQ ID NO: 29;
(i) the sense strand comprises a first nucleic acid sequence of SEQ ID NO: 28, and the antisense strand comprises a second nucleic acid sequence of SEQ ID NO: 30;
(j) the sense strand comprises a first nucleic acid sequence of SEQ ID NO: 28, and the antisense strand comprises a second nucleic acid sequence of SEQ ID NO: 31;
(k) the sense strand comprises a first nucleic acid sequence of SEQ ID NO: 28, and the antisense strand comprises a second nucleic acid sequence of SEQ ID NO: 32;
(l) the sense strand comprises a first nucleic acid sequence of SEQ ID NO: 13, and the antisense strand comprises a second nucleic acid sequence of SEQ ID NO: 33;
(m) the sense strand comprises a first nucleic acid sequence of SEQ ID NO: 34, and the antisense strand comprises a second nucleic acid sequence of SEQ ID NO: 35;
(n) the sense strand comprises a first nucleic acid sequence of SEQ ID NO: 34, and the antisense strand comprises a second nucleic acid sequence of SEQ ID NO: 36;
(o) the sense strand comprises a first nucleic acid sequence of SEQ ID NO: 34, and the antisense strand comprises a second nucleic acid sequence of SEQ ID NO: 37;
(p) the sense strand comprises a first nucleic acid sequence of SEQ ID NO: 34, and the antisense strand comprises a second nucleic acid sequence of SEQ ID NO: 38;
(q) the sense strand comprises a first nucleic acid sequence of SEQ ID NO: 21, and the antisense strand comprises a second nucleic acid sequence of SEQ ID NO: 39;
(r) the sense strand comprises a first nucleic acid sequence of SEQ ID NO: 40, and the antisense strand comprises a second nucleic acid sequence of SEQ ID NO: 41;
(s) the sense strand comprises a first nucleic acid sequence of SEQ ID NO: 40, and the antisense strand comprises a second nucleic acid sequence of SEQ ID NO: 42;
(t) the sense strand comprises a first nucleic acid sequence of SEQ ID NO: 40, and the antisense strand comprises a second nucleic acid sequence of SEQ ID NO: 43;
(u) the sense strand comprises a first nucleic acid sequence of SEQ ID NO: 40, and the antisense strand comprises a second nucleic acid sequence of SEQ ID NO: 44;
(v) the sense strand comprises a first nucleic acid sequence of SEQ ID NO: 17, and the antisense strand comprises a second nucleic acid sequence of SEQ ID NO: 45;
(w) the sense strand comprises a first nucleic acid sequence of SEQ ID NO: 19, and the antisense strand comprises a second nucleic acid sequence of SEQ ID NO: 46; and
(x) the sense strand comprises a first nucleic acid sequence of SEQ ID NO: 23, and the antisense strand comprises a second nucleic acid sequence of SEQ ID NO: 47.

In some embodiments, provided herein are SARM1 RNAi agents of Formula (I): R-L-D, wherein R is a dsRNA having a sense stand and an antisense strand, wherein the sense strand and the antisense strand form a duplex; wherein D is a delivery means (e.g., delivery moiety) for delivering the dsRNA into a cell; and wherein L is a linking means (e.g., linker) for linking the dsRNA to the delivery means, or optionally absent, wherein the sense strand and the antisense strand have a pair of nucleic acid sequences selected from the group consisting of:
(a) the sense strand has (e.g., consists of, or consists essentially of) a first nucleic acid sequence of SEQ ID NO: 13, and the antisense strand has (e.g., consists of, or consists essentially of) a second nucleic acid sequence of SEQ ID NO: 14;
(b) the sense strand has (e.g., consists of, or consists essentially of) a first nucleic acid sequence of SEQ ID NO: 15, and the antisense strand has (e.g., consists of, or consists essentially of) a second nucleic acid sequence of SEQ ID NO: 16;
(c) the sense strand has (e.g., consists of, or consists essentially of) a first nucleic acid sequence of SEQ ID NO: 17, and the antisense strand has (e.g., consists of, or consists essentially of) a second nucleic acid sequence of SEQ ID NO: 18;
(d) the sense strand has (e.g., consists of, or consists essentially of) a first nucleic acid sequence of SEQ ID NO: 19, and the antisense strand has (e.g., consists of, or consists essentially of) a second nucleic acid sequence of SEQ ID NO: 20;
(e) the sense strand has (e.g., consists of, or consists essentially of) a first nucleic acid sequence of SEQ ID NO: 21, and the antisense strand has (e.g., consists of, or consists essentially of) a second nucleic acid sequence of SEQ ID NO: 22; and
(f) the sense strand has (e.g., consists of, or consists essentially of) a first nucleic acid sequence of SEQ ID NO: 23, and the antisense strand has (e.g., consists of, or consists essentially of) a second nucleic acid sequence of SEQ ID NO: 24;
(e) the sense strand has (e.g., consists of, or consists essentially of) a first nucleic acid sequence of SEQ ID NO: 15, and the antisense strand has (e.g., consists of, or consists essentially of) a second nucleic acid sequence of SEQ ID NO: 27;
(f) the sense strand has (e.g., consists of, or consists essentially of) a first nucleic acid sequence of SEQ ID NO: 28, and the antisense strand has (e.g., consists of, or consists essentially of) a second nucleic acid sequence of SEQ ID NO: 29;
(g) the sense strand has (e.g., consists of, or consists essentially of) a first nucleic acid sequence of SEQ ID NO: 28, and the antisense strand has (e.g., consists of, or consists essentially of) a second nucleic acid sequence of SEQ ID NO: 30;
(h) the sense strand has (e.g., consists of, or consists essentially of) a first nucleic acid sequence of SEQ ID NO: 28, and the antisense strand has (e.g., consists of, or consists essentially of) a second nucleic acid sequence of SEQ ID NO: 31;
(i) the sense strand has (e.g., consists of, or consists essentially of) a first nucleic acid sequence of SEQ ID NO: 28, and the antisense strand has (e.g., consists of, or consists essentially of) a second nucleic acid sequence of SEQ ID NO: 32;
(j) the sense strand has (e.g., consists of, or consists essentially of) a first nucleic acid sequence of SEQ ID NO: 13, and the antisense strand has (e.g., consists of, or consists essentially of) a second nucleic acid sequence of SEQ ID NO: 33;
(k) the sense strand has (e.g., consists of, or consists essentially of) a first nucleic acid sequence of SEQ ID NO: 34, and the antisense strand has (e.g., consists of, or consists essentially of) a second nucleic acid sequence of SEQ ID NO: 35;
(l) the sense strand has (e.g., consists of, or consists essentially of) a first nucleic acid sequence of SEQ ID NO: 34, and the antisense strand has (e.g., consists of, or consists essentially of) a second nucleic acid sequence of SEQ ID NO: 36;
(m) the sense strand has (e.g., consists of, or consists essentially of) a first nucleic acid sequence of SEQ ID NO: 34, and the antisense strand has (e.g., consists of, or consists essentially of) a second nucleic acid sequence of SEQ ID NO: 37;
(n) the sense strand has (e.g., consists of, or consists essentially of) a first nucleic acid sequence of SEQ ID NO: 34, and the antisense strand has (e.g., consists of, or consists essentially of) a second nucleic acid sequence of SEQ ID NO: 38;
(o) the sense strand has (e.g., consists of, or consists essentially of) a first nucleic acid sequence of SEQ ID NO: 21, and the antisense strand has (e.g., consists of, or consists essentially of) a second nucleic acid sequence of SEQ ID NO: 39;
(p) the sense strand has (e.g., consists of, or consists essentially of) a first nucleic acid sequence of SEQ ID NO: 40, and the antisense strand has (e.g., consists of, or consists essentially of) a second nucleic acid sequence of SEQ ID NO: 41;
(q) the sense strand has (e.g., consists of, or consists essentially of) a first nucleic acid sequence of SEQ ID NO: 40, and the antisense strand has (e.g., consists of, or consists essentially of) a second nucleic acid sequence of SEQ ID NO: 42;
(r) the sense strand has (e.g., consists of, or consists essentially of) a first nucleic acid sequence of SEQ ID NO: 40, and the antisense strand has (e.g., consists of, or consists essentially of) a second nucleic acid sequence of SEQ ID NO: 43;
(s) the sense strand has (e.g., consists of, or consists essentially of) a first nucleic acid sequence of SEQ ID NO: 40, and the antisense strand has (e.g., consists of, or consists essentially of) a second nucleic acid sequence of SEQ ID NO: 44;
(t) the sense strand has (e.g., consists of, or consists essentially of) a first nucleic acid sequence of SEQ ID NO: 17, and the antisense strand has (e.g., consists of, or consists essentially of) a second nucleic acid sequence of SEQ ID NO: 45;
(u) the sense strand has (e.g., consists of, or consists essentially of) a first nucleic acid sequence of SEQ ID NO: 19, and the antisense strand has (e.g., consists of, or consists essentially of) a second nucleic acid sequence of SEQ ID NO: 46; and
(v) the sense strand has (e.g., consists of, or consists essentially of) a first nucleic acid sequence of SEQ ID NO: 23, and the antisense strand has (e.g., consists of, or consists essentially of) a second nucleic acid sequence of SEQ ID NO: 47.

In another aspect, provided herein are pharmaceutical compositions comprising a SARM1 RNAi agent described herein and a pharmaceutically acceptable carrier. Also provided herein are pharmaceutical compositions comprising a means for reducing SARM1 expression in a cell and a pharmaceutically acceptable carrier.

In another aspect, provided herein are methods of reducing SARM1 expression in a patient in need thereof, and such method comprises administering to the patient an effective amount of a SARM1 RNAi agent or a pharmaceutical composition described herein.

In another aspect, provided herein are methods of reducing axon degeneration in a patient in need thereof, and such method comprises administering to the patient an effective amount of a SARM1 RNAi agent or a pharmaceutical composition described herein.

In another aspect, provided herein are methods of treating a SARM1-mediated neurological disease in a patient in need thereof, and such method comprises administering to the patient an effective amount of the SARM1 RNAi agent or a pharmaceutical composition described herein.

Also provided herein are methods of reducing SARM1 expression in a cell (e.g., a neuron), and such methods can include introducing a SARM1 RNAi agent described herein into the cell; and incubating the cell for a time sufficient for degradation of SARM1 mRNA, thereby reducing SARM1 expression in the cell.

In another aspect, provided herein are SARM1 RNAi agents or pharmaceutical compositions comprising a SARM1 RNAi agent for use in reducing SARM1 expression. In another aspect, provided herein are SARM1 RNAi agents or pharmaceutical compositions comprising a SARM1 RNAi agent for use in reducing axon degeneration. Also provided herein are SARM1 RNAi agents or the pharmaceutical composition comprising a SARM1 RNAi agent for use in a therapy. Also provided are SARM1 RNAi agents or pharmaceutical compositions comprising a SARM1 RNAi agent for use in the treatment of a SARM1-mediated neurological disease. Also provided herein are uses of SARM1 RNAi agents in the manufacture of a medicament for reducing axon degeneration. Also provided herein are uses of SARM1 RNAi agents in the manufacture of a medicament for the treatment of a SARM1-mediated neurological disease.

### DETAILED DESCRIPTION

Provided herein are SARM1 RNAi agents and compositions comprising a SARM1 RNAi agent. Also provided herein are methods of using the SARM1 RNAi agents or compositions comprising a SARM1 RNAi agent for reducing SARM1 expression, reducing axon degeneration and/or treating SARM1-mediated neurological disease in a subject.

In some embodiments, provided herein are SARM1 RNAi agents having a sense strand and an antisense strand, and the sense strand and the antisense strand form a duplex. The antisense strand is complementary to a region of SARM1 mRNA. In a further embodiment, the sense strand and the antisense strand are each 15-30 nucleotides in length, e.g., 20-25 nucleotides in length. In some embodiments, provided herein are SARM1 RNAi agents having a sense strand of 21 nucleotides and an antisense strand of 23 nucleotides. In some embodiments, the sense strand and antisense strand of the SARM1 RNAi agent may have overhangs at either the 5′ end or the 3′ end (i.e., 5′ overhang or 3′ overhang). For example, the sense strand and the antisense strand may have 5′ or 3′ overhangs of 1 to 5 nucleotides or 1 to 3 nucleotides. In some embodiments, the antisense strand comprises a 3′ overhang of two nucleotides. In some embodiments, the sense strand and antisense strand sequences of the SARM1 RNAi agents are provided in Table 1.

Provided herein are SARM1 RNAi agents having a sense strand and an antisense strand, wherein the sense strand and the antisense strand form a duplex, wherein the sense strand and the antisense strand comprise a pair of nucleic acid sequences selected from the group consisting of:
(a) the sense strand comprises a first nucleic acid sequence having at least 90% sequence identity to SEQ ID NO: 1, and the antisense strand comprises a second nucleic acid sequence having at least 90% sequence identity to SEQ ID NO: 2;
(b) the sense strand comprises a first nucleic acid sequence having at least 90% sequence identity to SEQ ID NO: 3, and the antisense strand comprises a second nucleic acid sequence having at least 90% sequence identity to SEQ ID NO: 4;
(c) the sense strand comprises a first nucleic acid sequence having at least 90% sequence identity to SEQ ID NO: 5, and the antisense strand comprises a second nucleic acid sequence having at least 90% sequence identity to SEQ ID NO: 6;
(d) the sense strand comprises a first nucleic acid sequence having at least 90% sequence identity to SEQ ID NO: 7, and the antisense strand comprises a second nucleic acid sequence having at least 90% sequence identity to SEQ ID NO: 8;
(e) the sense strand comprises a first nucleic acid sequence having at least 90% sequence identity to SEQ ID NO: 9, and the antisense strand comprises a second nucleic acid sequence having at least 90% sequence identity to SEQ ID NO: 10; and
(f) the sense strand comprises a first nucleic acid sequence having at least 90% sequence identity to SEQ ID NO: 11, and the antisense strand comprises a second nucleic acid sequence having at least 90% sequence identity to SEQ ID NO: 12,
wherein optionally one or more nucleotides of the sense strand and the antisense strand are independently modified nucleotides, and wherein optionally one or more internucleotide linkages of the sense strand and the antisense strand are modified internucleotide linkages.

Provided herein are SARM1 RNAi agents having a sense strand and an antisense strand, wherein the sense strand and the antisense strand form a duplex, wherein the sense strand and the antisense strand comprise a pair of nucleic acid sequences selected from the group consisting of:
(a) the sense strand comprises a first nucleic acid sequence having at least 95% sequence identity to SEQ ID NO: 1, and the antisense strand comprises a second nucleic acid sequence having at least 95% sequence identity to SEQ ID NO: 2;
(b) the sense strand comprises a first nucleic acid sequence having at least 95% sequence identity to SEQ ID NO: 3, and the antisense strand comprises a second nucleic acid sequence having at least 95% sequence identity to SEQ ID NO: 4;
(c) the sense strand comprises a first nucleic acid sequence having at least 95% sequence identity to SEQ ID NO: 5, and the antisense strand comprises a second nucleic acid sequence having at least 95% sequence identity to SEQ ID NO: 6;
(d) the sense strand comprises a first nucleic acid sequence having at least 95% sequence identity to SEQ ID NO: 7, and the antisense strand comprises a second nucleic acid sequence having at least 95% sequence identity to SEQ ID NO: 8;
(e) the sense strand comprises a first nucleic acid sequence having at least 95% sequence identity to SEQ ID NO: 9, and the antisense strand comprises a second nucleic acid sequence having at least 95% sequence identity to SEQ ID NO: 10; and
(f) the sense strand comprises a first nucleic acid sequence having at least 95% sequence identity to SEQ ID NO: 11, and the antisense strand comprises a second nucleic acid sequence having at least 95% sequence identity to SEQ ID NO: 12, wherein optionally one or more nucleotides of the sense strand and the antisense strand are independently modified nucleotides, and wherein optionally one or more internucleotide linkages of the sense strand and the antisense strand are modified internucleotide linkages.

In some embodiments, provided herein are SARM1 RNAi agent having a sense strand and an antisense strand, wherein the sense strand and the antisense strand form a duplex, and wherein the sense strand comprises a first nucleic acid sequence having at least 95% sequence identity to SEQ ID NO: 1, and the antisense strand comprises a second nucleic acid sequence having at least 95% sequence identity to SEQ ID NO: 2, wherein one or more nucleotides of the sense strand and the antisense strand are independently modified nucleotides, and wherein one or more internucleotide linkages of the sense strand and the antisense strand are modified internucleotide linkages. In some embodiments, provided herein are SARM1 RNAi agent having a sense strand and an antisense strand, wherein the sense strand and the antisense strand form a duplex, and wherein the sense strand comprises a first nucleic acid sequence having at least 95% sequence identity to SEQ ID NO: 3, and the antisense strand comprises a second nucleic acid sequence having at least 95% sequence identity to SEQ ID NO: 4, wherein one or more nucleotides of the sense strand and the antisense strand are independently modified nucleotides, and wherein one or more internucleotide linkages of the sense strand and the antisense strand are modified internucleotide linkages. In some embodiments, provided herein are SARM1 RNAi agent having a sense strand and an antisense strand, wherein the sense strand and the antisense strand form a duplex, and wherein the sense strand comprises a first nucleic acid sequence having at least 95% sequence identity to SEQ ID NO: 5, and the antisense strand comprises a second nucleic acid sequence having at least 95% sequence identity to SEQ ID NO: 6, wherein one or more nucleotides of the sense strand and the antisense strand are independently modified nucleotides, and wherein one or more internucleotide linkages of the sense strand and the antisense strand are modified internucleotide linkages. In some embodiments, provided herein are SARM1 RNAi agent having a sense strand and an antisense strand, wherein the sense strand and the antisense strand form a duplex, and wherein the sense strand comprises a first nucleic acid sequence having at least 95% sequence identity to SEQ ID NO: 7, and the antisense strand comprises a second nucleic acid sequence having at least 95% sequence identity to SEQ ID NO: 8, wherein one or more nucleotides of the sense strand and the antisense strand are independently modified nucleotides, and wherein one or more internucleotide linkages of the sense strand and the antisense strand are modified internucleotide linkages. In some embodiments, provided herein are SARM1 RNAi agent having a sense strand and an antisense strand, wherein the sense strand and the antisense strand form a duplex, and wherein the sense strand comprises a first nucleic acid sequence having at least 95% sequence identity to SEQ ID NO: 9, and the antisense strand comprises a second nucleic acid sequence having at least 95% sequence identity to SEQ ID NO: 10, wherein one or more nucleotides of the sense strand and the antisense strand are independently modified nucleotides, and wherein one or more internucleotide linkages of the sense strand and the antisense strand are modified internucleotide linkages. In some embodiments, provided herein are SARM1 RNAi agent having a sense strand and an antisense strand, wherein the sense strand and the antisense strand form a duplex, and wherein the sense strand comprises a first nucleic acid sequence having at least 95% sequence identity to SEQ ID NO: 11, and the antisense strand comprises a second nucleic acid sequence having at least 95% sequence identity to SEQ ID NO: 12, wherein one or more nucleotides of the sense strand and the antisense strand are independently modified nucleotides, and wherein one or more internucleotide linkages of the sense strand and the antisense strand are modified internucleotide linkages.

TABLE 1
Unmodified Nucleic Acid Sequences of SARMI RNAi Agents

| | | | | | Start |
|---|---|---|---|---|---|
| | | | | | position of |
| | | | | | antisense |
| | | | | | strand target |
| SARM1 | | | | | region of |
| RNAi | | SEQ | | SEQ | human SARM1 |
| Agent | Sense Strand | ID | Antisense Strand | ID | transcript |
| No. | (5′ to 3′) | NO | (5′ to 3′) | NO | NM_015077.4* |
| 1 | GUUGCUCGACUCUAA | 1 | UAGCGGUUAGAGUCGAGC | 2 | 1333 |
| | CCGCUA | | AACGG | | |
| 2 | UUCGCCAACUAUUCU | 3 | UCACGUAGAAUAGUUGGC | 4 | 1763 |
| | ACGUGA | | GAAGG | | |
| 3 | ACCUUCGCCAACUAU | 5 | UGUAGAAUAGUUGGCGA | 6 | 1760 |
| | UCUACA | | AGGUCU | | |
| 4 | CGCCAACUAUUCUAC | 7 | UCGCACGUAGAAUAGUUG | 8 | 1765 |
| | GUGCGA | | GCGAA | | |
| 5 | CCGCAAGAGGUUCUU | 9 | UCCCUAAAGAACCUCUUG | 10 | 1723 |
| | UAGGGA | | CGGGU | | |
| 6 | AAACGCCUGGUUUCC | 11 | UGAGUAGGAAACCAGGCG | 12 | 1460 |
| | UACUCA | | UUUCA | | |

*Nucleotides 2-23 of the antisense strand are complementary to human SARM1 mRNA.

In some embodiments, the sense strand and the antisense strand of the SARM1 RNAi agent described herein comprise a pair of nucleic acid sequences selected from the group consisting of:
(a) the sense strand comprises a first nucleic acid sequence of SEQ ID NO: 1, and the antisense strand comprises a second nucleic acid sequence of SEQ TD NO: 2,
(b) the sense strand comprises a first nucleic acid sequence of SEQ ID NO: 3, and the antisense strand comprises a second nucleic acid sequence of SEQ TD NO: 4;
(c) the sense strand comprises a first nucleic acid sequence of SEQ ID NO: 5, and the antisense strand comprises a second nucleic acid sequence of SEQ TD NO: 6,
(d) the sense strand comprises a first nucleic acid sequence of SEQ ID NO: 7, and the antisense strand comprises a second nucleic acid sequence of SEQ TD NO: 8;
(e) the sense strand comprises a first nucleic acid sequence of SEQ ID NO: 9, and the antisense strand comprises a second nucleic acid sequence of SEQ ID NO: 10; and
(f) the sense strand comprises a first nucleic acid sequence of SEQ ID NO: 11, and the antisense strand comprises a second nucleic acid sequence of SEQ ID NO: 12,
wherein optionally one or more nucleotides of the sense strand and the antisense strand are independently modified nucleotides, and wherein optionally one or more internucleotide linkages of the sense strand and the antisense strand are modified internucleotide linkages.

In some embodiments, provided herein are SARM1 RNAi agent having a sense strand and an antisense strand, wherein the sense strand and the antisense strand form a duplex, and wherein the sense strand comprises a first nucleic acid sequence of SEQ ID NO: 1, and the antisense strand comprises a second nucleic acid sequence of SEQ ID NO: 2, wherein one or more nucleotides of the sense strand and the antisense strand are independently modified nucleotides, and wherein one or more internucleotide linkages of the sense strand and the antisense strand are modified internucleotide linkages. In some embodiments, provided herein are SARM1 RNAi agent having a sense strand and an antisense strand, wherein the sense strand and the antisense strand form a duplex, and wherein the sense strand comprises a first nucleic acid sequence of SEQ ID NO: 3, and the antisense strand comprises a second nucleic acid sequence of SEQ ID NO: 4, wherein one or more nucleotides of the sense strand and the antisense strand are independently modified nucleotides, and wherein one or more internucleotide linkages of the sense strand and the antisense strand are modified internucleotide linkages. In some embodiments, provided herein are SARM1 RNAi agent having a sense strand and an antisense strand, wherein the sense strand and the antisense strand form a duplex, and wherein the sense strand comprises a first nucleic acid sequence of SEQ ID NO: 5, and the antisense strand comprises a second nucleic acid sequence of SEQ ID NO: 6, wherein one or more nucleotides of the sense strand and the antisense strand are independently modified nucleotides, and wherein one or more internucleotide linkages of the sense strand and the antisense strand are modified internucleotide linkages. In some embodiments, provided herein are SARM1 RNAi agent having a sense strand and an antisense strand, wherein the sense strand and the antisense strand form a duplex, and wherein the sense strand comprises a first nucleic acid sequence of SEQ ID NO: 7, and the antisense strand comprises a second nucleic acid sequence of SEQ ID NO: 8, wherein one or more nucleotides of the sense strand and the antisense strand are independently modified nucleotides, and wherein one or more internucleotide linkages of the sense strand and the antisense strand are modified internucleotide linkages. In some embodiments, provided herein are SARM1 RNAi agent having a sense strand and an antisense strand, wherein the sense strand and the antisense strand form a duplex, and wherein the sense strand comprises a first nucleic acid sequence of SEQ ID NO: 9, and the antisense strand comprises a second nucleic acid sequence of SEQ ID NO: 10, wherein one or more nucleotides of the sense strand and the antisense strand are independently modified nucleotides, and wherein one or more internucleotide linkages of the sense strand and the antisense strand are modified internucleotide linkages. In some embodiments, provided herein are SARM1 RNAi agent having a sense strand and an antisense strand, wherein the sense strand and the antisense strand form a duplex, and wherein the sense strand comprises a first nucleic acid sequence of SEQ ID NO: 11, and the antisense strand comprises a second nucleic acid sequence of SEQ ID NO: 12, wherein one or more nucleotides of the sense strand and the antisense strand are independently modified nucleotides, and wherein one or more internucleotide linkages of the sense strand and the antisense strand are modified internucleotide linkages.

In some embodiments, the sense strand and the antisense strand of the SARM1 RNAi agent described herein have a pair of nucleic acid sequences selected from the group consisting of:
(a) the sense strand has (e.g., consists of, or consists essentially of) a first nucleic acid sequence of SEQ ID NO: 1, and the antisense strand has (e.g., consists of, or consists essentially of) a second nucleic acid sequence of SEQ ID NO: 2;
(b) the sense strand has (e.g., consists of, or consists essentially of) a first nucleic acid sequence of SEQ ID NO: 3, and the antisense strand has (e.g., consists of, or consists essentially of) a second nucleic acid sequence of SEQ ID NO: 4;
(c) the sense strand has (e.g., consists of, or consists essentially of) a first nucleic acid sequence of SEQ ID NO: 5, and the antisense strand has (e.g., consists of, or consists essentially of) a second nucleic acid sequence of SEQ ID NO: 6;
(d) the sense strand has (e.g., consists of, or consists essentially of) a first nucleic acid sequence of SEQ ID NO: 7, and the antisense strand has (e.g., consists of, or consists essentially of) a second nucleic acid sequence of SEQ ID NO: 8;
(e) the sense strand has (e.g., consists of, or consists essentially of) a first nucleic acid sequence of SEQ ID NO: 9, and the antisense strand has (e.g., consists of, or consists essentially of) a second nucleic acid sequence of SEQ ID NO: 10; and
(f) the sense strand has (e.g., consists of, or consists essentially of) a first nucleic acid sequence of SEQ ID NO: 11, and the antisense strand has (e.g., consists of, or consists essentially of) a second nucleic acid sequence of SEQ ID NO: 12,
wherein optionally one or more nucleotides of the sense strand and the antisense strand are independently modified nucleotides, and wherein optionally one or more internucleotide linkages of the sense strand and the antisense strand are modified internucleotide linkages.

In some embodiments, provided herein are SARM1 RNAi agent having a sense strand and an antisense strand, wherein the sense strand and the antisense strand form a duplex, and wherein the sense strand has a first nucleic acid sequence of SEQ ID NO: 1, and the antisense strand has a second nucleic acid sequence of SEQ ID NO: 2, wherein one or more nucleotides of the sense strand and the antisense strand are independently modified nucleotides, and wherein one or more internucleotide linkages of the sense strand and the antisense strand are modified internucleotide linkages. In some embodiments, provided herein are SARM1 RNAi agent having a sense strand and an antisense strand, wherein the sense strand and the antisense strand form a duplex, and wherein the sense strand has a first nucleic acid sequence of SEQ ID NO: 3, and the antisense strand has a second nucleic acid sequence of SEQ ID NO: 4, wherein one or more nucleotides of the sense strand and the antisense strand are independently modified nucleotides, and wherein one or more internucleotide linkages of the sense strand and the antisense strand are modified internucleotide linkages. In some embodiments, provided herein are SARM1 RNAi agent having a sense strand and an antisense strand, wherein the sense strand and the antisense strand form a duplex, and wherein the sense strand has a first nucleic acid sequence of SEQ ID NO: 5, and the antisense strand has a second nucleic acid sequence of SEQ ID NO: 6, wherein one or more nucleotides of the sense strand and the antisense strand are independently modified nucleotides, and wherein one or more internucleotide linkages of the sense strand and the antisense strand are modified internucleotide linkages. In some embodiments, provided herein are SARM1 RNAi agent having a sense strand and an antisense strand, wherein the sense strand and the antisense strand form a duplex, and wherein the sense strand has a first nucleic acid sequence of SEQ ID NO: 7, and the antisense strand has a second nucleic acid sequence of SEQ ID NO: 8, wherein one or more nucleotides of the sense strand and the antisense strand are independently modified nucleotides, and wherein one or more internucleotide linkages of the sense strand and the antisense strand are modified internucleotide linkages. In some embodiments, provided herein are SARM1 RNAi agent having a sense strand and an antisense strand, wherein the sense strand and the antisense strand form a duplex, and wherein the sense strand has a first nucleic acid sequence of SEQ ID NO: 9, and the antisense strand has a second nucleic acid sequence of SEQ ID NO: 10, wherein one or more nucleotides of the sense strand and the antisense strand are independently modified nucleotides, and wherein one or more internucleotide linkages of the sense strand and the antisense strand are modified internucleotide linkages. In some embodiments, provided herein are SARM1 RNAi agent having a sense strand and an antisense strand, wherein the sense strand and the antisense strand form a duplex, and wherein the sense strand has a first nucleic acid sequence of SEQ ID NO: 11, and the antisense strand has a second nucleic acid sequence of SEQ ID NO: 12, wherein one or more nucleotides of the sense strand and the antisense strand are independently modified nucleotides, and wherein one or more internucleotide linkages of the sense strand and the antisense strand are modified internucleotide linkages.

The SARM1 RNAi agents described herein may include modifications. The modifications can be made to one or more nucleotides of the sense strand and/or antisense strand or to the internucleotide linkages, which are the bonds between two nucleotides in the sense or antisense strand. For example, some 2′-modifications of ribose or deoxyribose can increase RNA or DNA stability and half-life. Such 2′-modifications can be 2′-fluoro, 2′-O-methyl (i.e., 2′-methoxy), 2′-O-alkyl, or 2′-O-methoxyethyl (2′-O-MOE).

In some embodiments, one or more nucleotides of the sense strand and/or the antisense strand are independently modified nucleotides, which means the sense strand and the antisense strand can have different modified nucleotides. In some embodiments, one or more nucleotides of the sense strand are modified nucleotides. In some embodiments, each nucleotide of the sense strand is a modified nucleotide. In some embodiments, one or more nucleotides of the antisense strand are modified nucleotides. In some embodiments, each nucleotide of the antisense strand is a modified nucleotide. In some embodiments, the modified nucleotide is a 2′-fluoro modified nucleotide, 2′-O-methyl modified nucleotide, or 2′-O-alkyl modified nucleotide, e.g., 2′-O—C₁₂₋₁₆ alkyl modified nucleotide. In some embodiments, each nucleotide of the sense strand and the antisense strand is independently a modified nucleotide, e.g., a 2′-fluoro modified nucleotide, 2′-O-methyl modified nucleotide, or 2′-O-alkyl modified nucleotide, e.g., 2′-O—C₁₂₋₁₆ alkyl modified nucleotide.

In some embodiments, the sense strand has four 2′-fluoro modified nucleotides, e.g., at positions 7, 9, 10, and 11 from the 5′ end of the sense strand. In some embodiments, nucleotides at positions other than positions 7, 9, 10, and 11 of the sense strand are 2′-O-methyl modified nucleotides. In some embodiments, the antisense strand has four 2′-fluoro modified nucleotides, e.g., at positions 2, 6, 14, and 16 from the 5′ end of the antisense strand. In some embodiments, nucleotides at positions other than positions 2, 6, 14 and 16 of the antisense strand are 2′-O-methyl modified nucleotides. In some embodiments, the sense strand has three 2′-fluoro modified nucleotides at positions 9, 10, and 11 from the 5′ end of the sense strand. In some embodiments, the nucleotides at positions other than positions 9, 10, and 11 of the sense strand are 2′-O-methyl modified nucleotides. In some embodiments, the antisense strand has five 2′-fluoro modified nucleotides at positions 2, 5, 7, 14, and 16 from the 5′ end of the antisense strand. In some embodiments, the nucleotides at positions other than positions 2, 5, 7, 14, and 16 of the antisense strand are 2′-O-methyl modified nucleotides. In some embodiments, the antisense strand has five 2′-fluoro modified nucleotides at positions 2, 5, 8, 14, and 16 from the 5′ end of the antisense strand. In some embodiments, the nucleotides at positions other than positions 2, 5, 8, 14, and 16 of the antisense strand are 2′-O-methyl modified nucleotides. In some embodiments, the antisense strand has five 2′-fluoro modified nucleotides at positions 2, 3, 7, 14, and 16 from the 5′ end of the antisense strand. In some embodiments, the nucleotides at positions other than positions 2, 3, 7, 14, and 16 of the antisense strand are 2′-O-methyl modified nucleotides. In some embodiments, the antisense strand has two 2′-fluoro modified nucleotides at positions 2 and 14 from the 5′ end of the antisense strand. In some embodiments, the nucleotides at positions other than positions 2 and 14 of the antisense strand are 2′-O-methyl modified nucleotides.

In some embodiments, the modified nucleotide is a 2′-O-alkyl modified nucleotide, e.g., 2′-O—C₁₂₋₁₆ alkyl modified nucleotide, which can serve as a delivery moiety. In some embodiments, the 2′-O-alkyl modified nucleotide is a 2′-O-hexadecyl uridine, 2′-O-hexadecyl cytidine, 2′-O-hexadecyl guanine, or 2′-O-hexadecyl adenosine. In some embodiments, 2′-O-hexadecyl uridine, 2′-O-hexadecyl cytidine, 2′-O-hexadecyl guanine, or 2′-O-hexadecyl adenosine is a modified nucleotide in the sense strand.

In some embodiments, the first nucleotide from the 5′ end of the antisense strand is a modified nucleotide that has a phosphate analog, e.g., 5′-vinylphosphonate (5′-VP). In some embodiments, the first nucleotide from the 5′ end of the antisense strand has a 5′ phosphate.

In some embodiments, the sense strand comprises an abasic moiety or inverted abasic moiety, e.g., a moiety shown in Table 3.

In some embodiments, the sense strand and the antisense strand have one or more modified internucleotide linkages. In some embodiments, the modified internucleotide linkage is phosphorothioate linkage. In some embodiments, the sense strand has four or five phosphorothioate linkages. In some embodiments, the antisense strand has four or five phosphorothioate linkages. In some embodiments, the sense strand and the antisense strand each has four or five phosphorothioate linkages. In some embodiments, the sense strand has four phosphorothioate linkages and the antisense strand has four phosphorothioate linkages.

In a further aspect, provided herein are SARM1 RNAi agent having a sense strand and an antisense strand, wherein the sense strand and the antisense strand form a duplex, wherein the sense strand and the antisense strand comprise a pair of nucleic acid sequences selected from the group consisting of:
(a) the sense strand comprises a first nucleic acid sequence of SEQ ID NO: 13, and the antisense strand comprises a second nucleic acid sequence of SEQ ID NO: 14;
(b) the sense strand comprises a first nucleic acid sequence of SEQ ID NO: 15, and the antisense strand comprises a second nucleic acid sequence of SEQ ID NO: 16;
(c) the sense strand comprises a first nucleic acid sequence of SEQ ID NO: 17, and the antisense strand comprises a second nucleic acid sequence of SEQ ID NO: 18;
(d) the sense strand comprises a first nucleic acid sequence of SEQ ID NO: 19, and the antisense strand comprises a second nucleic acid sequence of SEQ ID NO: 20;
(e) the sense strand comprises a first nucleic acid sequence of SEQ ID NO: 21, and the antisense strand comprises a second nucleic acid sequence of SEQ ID NO: 22;
(f) the sense strand comprises a first nucleic acid sequence of SEQ ID NO: 23, and the antisense strand comprises a second nucleic acid sequence of SEQ ID NO: 24;
(g) the sense strand comprises a first nucleic acid sequence of SEQ ID NO: 15, and the antisense strand comprises a second nucleic acid sequence of SEQ ID NO: 27;
(h) the sense strand comprises a first nucleic acid sequence of SEQ ID NO: 28, and the antisense strand comprises a second nucleic acid sequence of SEQ ID NO: 29;
(i) the sense strand comprises a first nucleic acid sequence of SEQ ID NO: 28, an the antisense strand comprises a second nucleic acid sequence of SEQ ID NO: 30;
(j) the sense strand comprises a first nucleic acid sequence of SEQ ID NO: 28, and the antisense strand comprises a second nucleic acid sequence of SEQ ID NO: 31;
(k) the sense strand comprises a first nucleic acid sequence of SEQ ID NO: 28, and the antisense strand comprises a second nucleic acid sequence of SEQ ID NO: 32;
(l) the sense strand comprises a first nucleic acid sequence of SEQ ID NO: 13, and the antisense strand comprises a second nucleic acid sequence of SEQ ID NO: 33;
(m) the sense strand comprises a first nucleic acid sequence of SEQ ID NO: 34, and the antisense strand comprises a second nucleic acid sequence of SEQ ID NO: 35;
(n) the sense strand comprises a first nucleic acid sequence of SEQ ID NO: 34, and the antisense strand comprises a second nucleic acid sequence of SEQ ID NO: 36;
(o) the sense strand comprises a first nucleic acid sequence of SEQ ID NO: 34, and the antisense strand comprises a second nucleic acid sequence of SEQ ID NO: 37;
(p) the sense strand comprises a first nucleic acid sequence of SEQ ID NO: 34, and the antisense strand comprises a second nucleic acid sequence of SEQ ID NO: 38;
(q) the sense strand comprises a first nucleic acid sequence of SEQ ID NO: 21, and the antisense strand comprises a second nucleic acid sequence of SEQ ID NO: 39;
(r) the sense strand comprises a first nucleic acid sequence of SEQ ID NO: 40, and the antisense strand comprises a second nucleic acid sequence of SEQ ID NO: 41;
(s) the sense strand comprises a first nucleic acid sequence of SEQ ID NO: 40, and the antisense strand comprises a second nucleic acid sequence of SEQ ID NO: 42;
(t) the sense strand comprises a first nucleic acid sequence of SEQ ID NO: 40, and the antisense strand comprises a second nucleic acid sequence of SEQ ID NO: 43;
(u) the sense strand comprises a first nucleic acid sequence of SEQ ID NO: 40, and the antisense strand comprises a second nucleic acid sequence of SEQ ID NO: 44;
(v) the sense strand comprises a first nucleic acid sequence of SEQ ID NO: 17, and the antisense strand comprises a second nucleic acid sequence of SEQ ID NO: 45;
(w) the sense strand comprises a first nucleic acid sequence of SEQ ID NO: 19, and the antisense strand comprises a second nucleic acid sequence of SEQ ID NO: 46; and
(x) the sense strand comprises a first nucleic acid sequence of SEQ ID NO: 23, and the antisense strand comprises a second nucleic acid sequence of SEQ ID NO: 47.

In some embodiments, provided herein are SARM1 RNAi agent having a sense strand and an antisense strand, wherein the sense strand and the antisense strand form a duplex, and wherein the sense strand comprises a first nucleic acid sequence of SEQ ID NO: 13, and the antisense strand comprises a second nucleic acid sequence of SEQ ID NO: 14. In some embodiments, provided herein are SARM1 RNAi agent having a sense strand and an antisense strand, wherein the sense strand and the antisense strand form a duplex, and wherein the sense strand comprises a first nucleic acid sequence of SEQ ID NO: 15, and the antisense strand comprises a second nucleic acid sequence of SEQ ID NO: 16. In some embodiments, provided herein are SARM1 RNAi agent having a sense strand and an antisense strand, wherein the sense strand and the antisense strand form a duplex, and wherein the sense strand comprises a first nucleic acid sequence of SEQ ID NO: 17, and the antisense strand comprises a second nucleic acid sequence of SEQ ID NO: 18. In some embodiments, provided herein are SARM1 RNAi agent having a sense strand and an antisense strand, wherein the sense strand and the antisense strand form a duplex, and wherein the sense strand comprises a first nucleic acid sequence of SEQ ID NO: 19, and the antisense strand comprises a second nucleic acid sequence of SEQ ID NO: 20. In some embodiments, provided herein are SARM1 RNAi agent having a sense strand and an antisense strand, wherein the sense strand and the antisense strand form a duplex, and wherein the sense strand comprises a first nucleic acid sequence of SEQ ID NO: 21, and the antisense strand comprises a second nucleic acid sequence of SEQ ID NO: 22. In some embodiments, provided herein are SARM1 RNAi agent having a sense strand and an antisense strand, wherein the sense strand and the antisense strand form a duplex, and wherein the sense strand comprises a first nucleic acid sequence of SEQ ID NO: 23, and the antisense strand comprises a second nucleic acid sequence of SEQ ID NO: 24. In some embodiments, provided herein are SARM1 RNAi agent having a sense strand and an antisense strand, wherein the sense strand and the antisense strand form a duplex, and wherein the sense strand comprises a first nucleic acid sequence of SEQ ID NO: 15, and the antisense strand comprises a second nucleic acid sequence of SEQ ID NO: 27. In some embodiments, provided herein are SARM1 RNAi agent having a sense strand and an antisense strand, wherein the sense strand and the antisense strand form a duplex, and wherein the sense strand comprises a first nucleic acid sequence of SEQ ID NO: 28, and the antisense strand comprises a second nucleic acid sequence of SEQ ID NO: 29. In some embodiments, provided herein are SARM1 RNAi agent having a sense strand and an antisense strand, wherein the sense strand and the antisense strand form a duplex, and wherein the sense strand comprises a first nucleic acid sequence of SEQ ID NO: 28, and the antisense strand comprises a second nucleic acid sequence of SEQ ID NO: 30. In some embodiments, provided herein are SARM1 RNAi agent having a sense strand and an antisense strand, wherein the sense strand and the antisense strand form a duplex, and wherein the sense strand comprises a first nucleic acid sequence of SEQ ID NO: 28, and the antisense strand comprises a second nucleic acid sequence of SEQ ID NO: 31. In some embodiments, provided herein are SARM1 RNAi agent having a sense strand and an antisense strand, wherein the sense strand and the antisense strand form a duplex, and wherein the sense strand comprises a first nucleic acid sequence of SEQ ID NO: 28, and the antisense strand comprises a second nucleic acid sequence of SEQ ID NO: 32. In some embodiments, provided herein are SARM1 RNAi agent having a sense strand and an antisense strand, wherein the sense strand and the antisense strand form a duplex, and wherein the sense strand comprises a first nucleic acid sequence of SEQ ID NO: 13, and the antisense strand comprises a second nucleic acid sequence of SEQ ID NO: 33. In some embodiments, provided herein are SARM1 RNAi agent having a sense strand and an antisense strand, wherein the sense strand and the antisense strand form a duplex, and wherein the sense strand comprises a first nucleic acid sequence of SEQ ID NO: 34, and the antisense strand comprises a second nucleic acid sequence of SEQ ID NO: 35. In some embodiments, provided herein are SARM1 RNAi agent having a sense strand and an antisense strand, wherein the sense strand and the antisense strand form a duplex, and wherein the sense strand comprises a first nucleic acid sequence of SEQ ID NO: 34, and the antisense strand comprises a second nucleic acid sequence of SEQ ID NO: 36. In some embodiments, provided herein are SARM1 RNAi agent having a sense strand and an antisense strand, wherein the sense strand and the antisense strand form a duplex, and wherein the sense strand comprises a first nucleic acid sequence of SEQ ID NO: 34, and the antisense strand comprises a second nucleic acid sequence of SEQ ID NO: 37. In some embodiments, provided herein are SARM1 RNAi agent having a sense strand and an antisense strand, wherein the sense strand and the antisense strand form a duplex, and wherein the sense strand comprises a first nucleic acid sequence of SEQ ID NO: 34, and the antisense strand comprises a second nucleic acid sequence of SEQ ID NO: 38. In some embodiments, provided herein are SARM1 RNAi agent having a sense strand and an antisense strand, wherein the sense strand and the antisense strand form a duplex, and wherein the sense strand comprises a first nucleic acid sequence of SEQ ID NO: 21, and the antisense strand comprises a second nucleic acid sequence of SEQ ID NO: 39. In some embodiments, provided herein are SARM1 RNAi agent having a sense strand and an antisense strand, wherein the sense strand and the antisense strand form a duplex, and wherein the sense strand comprises a first nucleic acid sequence of SEQ ID NO: 40, and the antisense strand comprises a second nucleic acid sequence of SEQ ID NO: 41. In some embodiments, provided herein are SARM1 RNAi agent having a sense strand and an antisense strand, wherein the sense strand and the antisense strand form a duplex, and wherein the sense strand comprises a first nucleic acid sequence of SEQ ID NO: 40, and the antisense strand comprises a second nucleic acid sequence of SEQ ID NO: 42. In some embodiments, provided herein are SARM1 RNAi agent having a sense strand and an antisense strand, wherein the sense strand and the antisense strand form a duplex, and wherein the sense strand comprises a first nucleic acid sequence of SEQ ID NO: 40, and the antisense strand comprises a second nucleic acid sequence of SEQ ID NO: 43. In some embodiments, provided herein are SARM1 RNAi agent having a sense strand and an antisense strand, wherein the sense strand and the antisense strand form a duplex, and wherein the sense strand comprises a first nucleic acid sequence of SEQ ID NO: 40, and the antisense strand comprises a second nucleic acid sequence of SEQ ID NO: 44. In some embodiments, provided herein are SARM1 RNAi agent having a sense strand and an antisense strand, wherein the sense strand and the antisense strand form a duplex, and wherein the sense strand comprises a first nucleic acid sequence of SEQ ID NO: 17, and the antisense strand comprises a second nucleic acid sequence of SEQ ID NO: 45. In some embodiments, provided herein are SARM1 RNAi agent having a sense strand and an antisense strand, wherein the sense strand and the antisense strand form a duplex, and wherein the sense strand comprises a first nucleic acid sequence of SEQ ID NO: 19, and the antisense strand comprises a second nucleic acid sequence of SEQ ID NO: 46. In some embodiments, provided herein are SARM1 RNAi agent having a sense strand and an antisense strand, wherein the sense strand and the antisense strand form a duplex, and wherein the sense strand comprises a first nucleic acid sequence of SEQ ID NO: 23, and the antisense strand comprises a second nucleic acid sequence of SEQ ID NO: 47.

In some embodiments, provided herein are SARM1 RNAi agent having a sense strand and an antisense strand, wherein the sense strand and the antisense strand form a duplex, wherein the sense strand and the antisense strand have a pair of nucleic acid sequences selected from the group consisting of:
(a) the sense strand has (e.g., consists of, or consists essentially of) a first nucleic acid sequence of SEQ ID NO: 13, and the antisense strand has (e.g., consists of, or consists essentially of) a second nucleic acid sequence of SEQ ID NO: 14;
(b) the sense strand has (e.g., consists of, or consists essentially of) a first nucleic acid sequence of SEQ ID NO: 15, and the antisense strand has (e.g., consists of, or consists essentially of) a second nucleic acid sequence of SEQ ID NO: 16;
(c) the sense strand has (e.g., consists of, or consists essentially of) a first nucleic acid sequence of SEQ ID NO: 17, and the antisense strand has (e.g., consists of, or consists essentially of) a second nucleic acid sequence of SEQ ID NO: 18;
(d) the sense strand has (e.g., consists of, or consists essentially of) a first nucleic acid sequence of SEQ ID NO: 19, and the antisense strand has (e.g., consists of, or consists essentially of) a second nucleic acid sequence of SEQ ID NO: 20;
(e) the sense strand has (e.g., consists of, or consists essentially of) a first nucleic acid sequence of SEQ ID NO: 21, and the antisense strand has (e.g., consists of, or consists essentially of) a second nucleic acid sequence of SEQ ID NO: 22;
(f) the sense strand has (e.g., consists of, or consists essentially of) a first nucleic acid sequence of SEQ ID NO: 23, and the antisense strand has (e.g., consists of, or consists essentially of) a second nucleic acid sequence of SEQ ID NO: 24;
(g) the sense strand has (e.g., consists of, or consists essentially of) a first nucleic acid sequence of SEQ ID NO: 15, and the antisense strand has (e.g., consists of, or consists essentially of) a second nucleic acid sequence of SEQ ID NO: 27;
(h) the sense strand has (e.g., consists of, or consists essentially of) a first nucleic acid sequence of SEQ ID NO: 28, and the antisense strand has (e.g., consists of, or consists essentially of) a second nucleic acid sequence of SEQ ID NO: 29;
(i) the sense strand has (e.g., consists of, or consists essentially of) a first nucleic acid sequence of SEQ ID NO: 28, and the antisense strand has (e.g., consists of, or consists essentially of) a second nucleic acid sequence of SEQ ID NO: 30;
(j) the sense strand has (e.g., consists of, or consists essentially of) a first nucleic acid sequence of SEQ ID NO: 28, and the antisense strand has (e.g., consists of, or consists essentially of) a second nucleic acid sequence of SEQ ID NO: 31;
(k) the sense strand has (e.g., consists of, or consists essentially of) a first nucleic acid
(l) sequence of SEQ ID NO: 28, and the antisense strand has (e.g., consists of, or consists essentially of) a second nucleic acid sequence of SEQ ID NO: 32;
(m) the sense strand has (e.g., consists of, or consists essentially of) a first nucleic acid sequence of SEQ ID NO: 13, and the antisense strand has (e.g., consists of, or consists essentially of) a second nucleic acid sequence of SEQ ID NO: 33;
(n) the sense strand has (e.g., consists of, or consists essentially of) a first nucleic acid sequence of SEQ ID NO: 34, and the antisense strand has (e.g., consists of, or consists essentially of) a second nucleic acid sequence of SEQ ID NO: 35;
(o) the sense strand has (e.g., consists of, or consists essentially of) a first nucleic acid sequence of SEQ ID NO: 34, and the antisense strand has (e.g., consists of, or consists essentially of) a second nucleic acid sequence of SEQ ID NO: 36;
(p) the sense strand has (e.g., consists of, or consists essentially of) a first nucleic acid sequence of SEQ ID NO: 34, and the antisense strand has (e.g., consists of, or consists essentially of) a second nucleic acid sequence of SEQ ID NO: 37;
(q) the sense strand has (e.g., consists of, or consists essentially of) a first nucleic acid sequence of SEQ ID NO: 34, and the antisense strand has (e.g., consists of, or consists essentially of) a second nucleic acid sequence of SEQ ID NO: 38;
(r) the sense strand has (e.g., consists of, or consists essentially of) a first nucleic acid sequence of SEQ ID NO: 21, and the antisense strand has (e.g., consists of, or consists essentially of) a second nucleic acid sequence of SEQ ID NO: 39;
(s) the sense strand has (e.g., consists of, or consists essentially of) a first nucleic acid sequence of SEQ ID NO: 40, and the antisense strand has (e.g., consists of, or consists essentially of) a second nucleic acid sequence of SEQ ID NO: 41;
(t) the sense strand has (e.g., consists of, or consists essentially of) a first nucleic acid sequence of SEQ ID NO: 40, and the antisense strand has (e.g., consists of, or consists essentially of) a second nucleic acid sequence of SEQ ID NO: 42;
(u) the sense strand has (e.g., consists of, or consists essentially of) a first nucleic acid sequence of SEQ ID NO: 40, and the antisense strand has (e.g., consists of, or consists essentially of) a second nucleic acid sequence of SEQ ID NO: 43;
(v) the sense strand has (e.g., consists of, or consists essentially of) a first nucleic acid sequence of SEQ ID NO: 40, and the antisense strand has (e.g., consists of, or consists essentially of) a second nucleic acid sequence of SEQ ID NO: 44;
(v) the sense strand has (e.g., consists of, or consists essentially of) a first nucleic acid sequence of SEQ ID NO: 17, and the antisense strand has (e.g., consists of, or consists essentially of) a second nucleic acid sequence of SEQ ID NO: 45;
(w) the sense strand has (e.g., consists of, or consists essentially of) a first nucleic acid sequence of SEQ ID NO: 19, and the antisense strand has (e.g., consists of, or consists essentially of) a second nucleic acid sequence of SEQ ID NO: 46; and
(x) the sense strand has (e.g., consists of, or consists essentially of) a first nucleic acid sequence of SEQ ID NO: 23, and the antisense strand has (e.g., consists of, or consists essentially of) a second nucleic acid sequence of SEQ ID NO: 47.

In some embodiments, provided herein are SARM1 RNAi agent having a sense strand and an antisense strand, wherein the sense strand and the antisense strand form a duplex, and wherein the sense strand has a first nucleic acid sequence of SEQ ID NO: 13, and the antisense strand has a second nucleic acid sequence of SEQ ID NO: 14. In some embodiments, provided herein are SARM1 RNAi agent having a sense strand and an antisense strand, wherein the sense strand and the antisense strand form a duplex, and wherein the sense strand has a first nucleic acid sequence of SEQ ID NO: 15, and the antisense strand has a second nucleic acid sequence of SEQ ID NO: 16. In some embodiments, provided herein are SARM1 RNAi agent having a sense strand and an antisense strand, wherein the sense strand and the antisense strand form a duplex, and wherein the sense strand has a first nucleic acid sequence of SEQ ID NO: 17, and the antisense strand has a second nucleic acid sequence of SEQ ID NO: 18. In some embodiments, provided herein are SARM1 RNAi agent having a sense strand and an antisense strand, wherein the sense strand and the antisense strand form a duplex, and wherein the sense strand has a first nucleic acid sequence of SEQ ID NO: 19, and the antisense strand has a second nucleic acid sequence of SEQ ID NO: 20. In some embodiments, provided herein are SARM1 RNAi agent having a sense strand and an antisense strand, wherein the sense strand and the antisense strand form a duplex, and wherein the sense strand has a first nucleic acid sequence of SEQ ID NO: 21, and the antisense strand has a second nucleic acid sequence of SEQ ID NO: 22. In some embodiments, provided herein are SARM1 RNAi agent having a sense strand and an antisense strand, wherein the sense strand and the antisense strand form a duplex, and wherein the sense strand has a first nucleic acid sequence of SEQ ID NO: 23, and the antisense strand has a second nucleic acid sequence of SEQ ID NO: 24. In some embodiments, provided herein are SARM1 RNAi agent having a sense strand and an antisense strand, wherein the sense strand and the antisense strand form a duplex, and wherein the sense strand has a first nucleic acid sequence of SEQ ID NO: 15, and the antisense strand has a second nucleic acid sequence of SEQ ID NO: 27. In some embodiments, provided herein are SARM1 RNAi agent having a sense strand and an antisense strand, wherein the sense strand and the antisense strand form a duplex, and wherein the sense strand has a first nucleic acid sequence of SEQ ID NO: 28, and the antisense strand has a second nucleic acid sequence of SEQ ID NO: 29. In some embodiments, provided herein are SARM1 RNAi agent having a sense strand and an antisense strand, wherein the sense strand and the antisense strand form a duplex, and wherein the sense strand has a first nucleic acid sequence of SEQ ID NO: 28, and the antisense strand has a second nucleic acid sequence of SEQ ID NO: 30. In some embodiments, provided herein are SARM1 RNAi agent having a sense strand and an antisense strand, wherein the sense strand and the antisense strand form a duplex, and wherein the sense strand has a first nucleic acid sequence of SEQ ID NO: 28, and the antisense strand has a second nucleic acid sequence of SEQ ID NO: 31. In some embodiments, provided herein are SARM1 RNAi agent having a sense strand and an antisense strand, wherein the sense strand and the antisense strand form a duplex, and wherein the sense strand has a first nucleic acid sequence of SEQ ID NO: 28, and the antisense strand has a second nucleic acid sequence of SEQ ID NO: 32. In some embodiments, provided herein are SARM1 RNAi agent having a sense strand and an antisense strand, wherein the sense strand and the antisense strand form a duplex, and wherein the sense strand has a first nucleic acid sequence of SEQ ID NO: 13, and the antisense strand has a second nucleic acid sequence of SEQ ID NO: 33. In some embodiments, provided herein are SARM1 RNAi agent having a sense strand and an antisense strand, wherein the sense strand and the antisense strand form a duplex, and wherein the sense strand has a first nucleic acid sequence of SEQ ID NO: 34, and the antisense strand has a second nucleic acid sequence of SEQ ID NO: 35. In some embodiments, provided herein are SARM1 RNAi agent having a sense strand and an antisense strand, wherein the sense strand and the antisense strand form a duplex, and wherein the sense strand has a first nucleic acid sequence of SEQ ID NO: 34, and the antisense strand has a second nucleic acid sequence of SEQ ID NO: 36. In some embodiments, provided herein are SARM1 RNAi agent having a sense strand and an antisense strand, wherein the sense strand and the antisense strand form a duplex, and wherein the sense strand has a first nucleic acid sequence of SEQ ID NO: 34, and the antisense strand has a second nucleic acid sequence of SEQ ID NO: 37. In some embodiments, provided herein are SARM1 RNAi agent having a sense strand and an antisense strand, wherein the sense strand and the antisense strand form a duplex, and wherein the sense strand has a first nucleic acid sequence of SEQ ID NO: 34, and the antisense strand has a second nucleic acid sequence of SEQ ID NO: 38. In some embodiments, provided herein are SARM1 RNAi agent having a sense strand and an antisense strand, wherein the sense strand and the antisense strand form a duplex, and wherein the sense strand has a first nucleic acid sequence of SEQ ID NO: 21, and the antisense strand has a second nucleic acid sequence of SEQ ID NO: 39. In some embodiments, provided herein are SARM1 RNAi agent having a sense strand and an antisense strand, wherein the sense strand and the antisense strand form a duplex, and wherein the sense strand has a first nucleic acid sequence of SEQ ID NO: 40, and the antisense strand has a second nucleic acid sequence of SEQ ID NO: 41. In some embodiments, provided herein are SARM1 RNAi agent having a sense strand and an antisense strand, wherein the sense strand and the antisense strand form a duplex, and wherein the sense strand has a first nucleic acid sequence of SEQ ID NO: 40, and the antisense strand has a second nucleic acid sequence of SEQ ID NO: 42. In some embodiments, provided herein are SARM1 RNAi agent having a sense strand and an antisense strand, wherein the sense strand and the antisense strand form a duplex, and wherein the sense strand has a first nucleic acid sequence of SEQ ID NO: 40, and the antisense strand has a second nucleic acid sequence of SEQ ID NO: 43. In some embodiments, provided herein are SARM1 RNAi agent having a sense strand and an antisense strand, wherein the sense strand and the antisense strand form a duplex, and wherein the sense strand has a first nucleic acid sequence of SEQ ID NO: 40, and the antisense strand has a second nucleic acid sequence of SEQ ID NO: 44. In some embodiments, provided herein are SARM1 RNAi agent having a sense strand and an antisense strand, wherein the sense strand and the antisense strand form a duplex, and wherein the sense strand has a first nucleic acid sequence of SEQ ID NO: 17, and the antisense strand has a second nucleic acid sequence of SEQ ID NO: 45. In some embodiments, provided herein are SARM1 RNAi agent having a sense strand and an antisense strand, wherein the sense strand and the antisense strand form a duplex, and wherein the sense strand has a first nucleic acid sequence of SEQ ID NO: 19, and the antisense strand has a second nucleic acid sequence of SEQ ID NO: 46. In some embodiments, provided herein are SARM1 RNAi agent having a sense strand and an antisense strand, wherein the sense strand and the antisense strand form a duplex, and wherein the sense strand has a first nucleic acid sequence of SEQ ID NO: 23, and the antisense strand has a second nucleic acid sequence of SEQ ID NO: 47.

TABLE 2
SARM1 RNAi Agents with Modifications

| | | | SEQ |
|---|---|---|---|
| SARM1 RNAi | | | ID |
| Agent No. | Strand | Sequence from 5′ to 3′ end | NO |
| 13 | S | mG*mU*mUmGmCmUfCmGfAfCfUmCmUmAmAmCmCmGmC*mU*mA | 13 |
| | AS | VPmU*fA*mGmCmGfGmUmUmAmGmAmGmUfCmGfAmGmCmAmAmC*mG*mG | 14 |
| 14 | S | mU*mU*mCmGmCmCfAmAfCfUfAmUmUmCmUmAmCmGmU*mG*mA | 15 |
| | AS | VPmU*fC*mAmCmGfUmAmGmAmAmUmAmGfUmUfGmGmCmGmAmA*mG*mG | 16 |
| 15 | S | mA*mC*mCmUmUmCfGmCfCfAfAmCmUmAmUmUmCmUmA*mC*mA | 17 |
| | AS | VPmU*fG*mUmAmGfAmAmUmAmGmUmUmGfGmCfGmAmAmGmGmU*mC*mU | 18 |
| 16 | S | mC*mG*mCmCmAmAfCmUfAfUfUmCmUmAmCmGmUmGmC*mG*mA | 19 |
| | AS | VPmU*fC*mGmCmAfCmGmUmAmGmAmAmUfAmGfUmUmGmGmCmG*mA*mA | 20 |
| 17 | S | mC*mC*mGmCmAmAfGmAfGfGfUmUmCmUmUmUmAmGmG*mG*mA | 21 |
| | AS | VPmU*fC*mCmCmUfAmAmAmGmAmAmCmCfUmCfUmUmGmCmGmG*mG*mU | 22 |
| 18 | S | mA*mA*mAmCmGmCfCmUfGfGfUmUmUmCmCmUmAmCmU*mC*mA | 23 |
| | AS | VPmU*fG*mAmGmUfAmGmGmAmAmAmCmCfAmGfGmCmGmUmUmU*mC*mA | 24 |
| 19 | S | mU*mU*mCmGmCmCmAmAfCfUfAmUmUmCmUmAmCmGmU*mG*mA | 28 |
| | AS | mU*fC*mAmCfGmUfAmGmAmAmUmAmGfUmUfGmGmCmGmAmA*mG*mG | 29 |
| 20 | S | mU*mU*mCmGmCmCmAmAfCfUfAmUmUmCmUmAmCmGmU*mG*mA | 28 |
| | AS | mU*fC*fAmCmGmUfAmGmAmAmUmAmGfUmUfGmGmCmGmAmA*mG*mG | 30 |
| 21 | S | mU*mU*mCmGmCmCmAmAfCfUfAmUmUmCmUmAmCmGmU*mG*mA | 28 |
| | AS | mU*fC*mAmCfGmUmAfGmAmAmUmAmGfUmUfGmGmCmGmAmA*mG*mG | 31 |
| 22 | S | mU*mU*mCmGmCmCmAmAfCfUfAmUmUmCmUmAmCmGmU*mG*mA | 28 |
| | AS | mU*fC*mAmCmGmUmAmGmAmAmUmAmGfUmUmGmGmCmGmAmA*mG*mG | 32 |
| 23 | S | mG*mU*mUmGmCmUmCmGfAfCfUmCmUmAmAmCmCmGmC*mU*mA | 34 |
| | AS | mU*fA*mGmCfGmGfUmUmAmGmAmGmUfCmGfAmGmCmAmAmC*mG*mG | 35 |
| 24 | S | mG*mU*mUmGmCmUmCmGfAfCfUmCmUmAmAmCmCmGmC*mU*mA | 34 |
| | AS | mU*fA*fGmCmGmGfUmUmAmGmAmGmUfCmGfAmGmCmAmAmC*mG*mG | 36 |
| 25 | S | mG*mU*mUmGmCmUmCmGfAfCfUmCmUmAmAmCmCmGmC*mU*mA | 34 |
| | AS | mU*fA*mGmCfGmGmUfUmAmGmAmGmUfCmGfAmGmCmAmAmC*mG*mG | 37 |
| 26 | S | mG*mU*mUmGmCmUmCmGfAfCfUmCmUmAmAmCmCmGmC*mU*mA | 34 |
| | AS | mU*fA*mGmCmGmGmUmUmAmGmAmGmUfCmGmAmGmCmAmAmC*mG*mG | 38 |
| 27 | S | mC*mC*mGmCmAmAmGmAfGfGfUmUmCmUmUmUmAmGmG*mG*mA | 40 |
| | AS | mU*fC*mCmCfUmAfAmAmGmAmAmCmCfUmCfUmUmGmCmGmG*mG*mU | 41 |
| 28 | S | mC*mC*mGmCmAmAmGmAfGfGfUmUmCmUmUmUmAmGmG*mG*mA | 40 |
| | AS | mU*fC*fCmCmUmAfAmAmGmAmAmCmCfUmCfUmUmGmCmGmG*mG*mU | 42 |
| 29 | S | mC*mC*mGmCmAmAmGmAfGfGfUmUmCmUmUmUmAmGmG*mG*mA | 40 |
| | AS | mU*fC*mCmCfUmAmAfAmGmAmAmCmCfUmCfUmUmGmCmGmG*mG*mU | 43 |
| 30 | S | mC*mC*mGmCmAmAmGmAfGfGfUmUmCmUmUmUmAmGmG*mG*mA | 40 |
| | AS | mU*fC*mCmCmUmAmAmAmGmAmAmCmCfUmCmUmUmGmCmGmG*mG*mU | 44 |
| 7 | S | mG*mU*mUmGmCmUfCmGfAfCfUmCmUmAmAmCmCmGmC*mU*mA | 13 |
| | AS | mU*fA*mGmCmGfGmUmUmAmGmAmGmUfCmGfAmGmCmAmAmC*mG*mG | 33 |
| 8 | S | mU*mU*mCmGmCmCfAmAfCfUfAmUmUmCmUmAmCmGmU*mG*mA | 15 |
| | AS | mU*fC*mAmCmGfUmAmGmAmAmUmAmGfUmUfGmGmCmGmAmA*mG*mG | 27 |
| 9 | S | mA*mC*mCmUmUmCfGmCfCfAfAmCmUmAmUmUmCmUmA*mC*mA | 17 |
| | AS | mU*fG*mUmAmGfAmAmUmAmGmUmUmGfGmCfGmAmAmGmGmU*mC*mU | 45 |
| 10 | S | mC*mG*mCmCmAmAfCmUfAfUfUmCmUmAmCmGmUmGmC*mG*mA | 19 |
| | AS | mU*fC*mGmCmAfCmGmUmAmGmAmAmUfAmGfUmUmGmGmCmG*mA*mA | 46 |
| 11 | S | mC*mC*mGmCmAmAfGmAfGfGfUmUmCmUmUmUmAmGmG*mG*mA | 21 |
| | AS | mU*fC*mCmCmUfAmAmAmGmAmAmCmCfUmCfUmUmGmCmGmG*mG*mU | 39 |
| 12 | S | mA*mA*mAmCmGmCfCmUfGfGfUmUmUmCmCmUmAmCmU*mC*mA | 23 |
| | AS | mU*fG*mAmGmUfAmGmGmAmAmAmCmCfAmGfGmCmGmUmUmU*mC*mA | 47 |

Abbreviations-“m” indicates 2′-OMe; “f” indicates 2′-fluoro; “*” indicates phosphorothioate linkage; “VP” indicates 5′-vinylphosphonate; “S” means the sense strand; “AS” means the antisense strand; unless otherwise noted, the 5′ position of the AS can include 5′-phosphate or 5′-vinylphosphonate.

TABLE 3
Abasic or inverted abasic (iAb) moieties

| | Structure |
|---|---|
| 1 (abasic) | 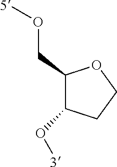 |
| 2 (iAb) | 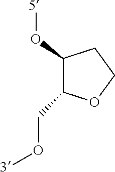 |

“5′” and “3′” indicate the 5′ to 3′ direction of the sequences.

In some embodiments, the sense strand of the SARM1 RNAi agent has a delivery moiety. In some embodiments, the sense strand of the SARM1 RNAi agent has a delivery moiety conjugated to the 3′ end of the sense strand. In some embodiments, the sense strand of the SARM1 RNAi agent has a delivery moiety conjugated to the 5′ end of the sense strand. In some embodiments, the sense strand of the SARM1 RNAi agent has delivery moieties conjugated to both the 5′ end and the 3′ end of the sense strand. In some embodiments, the sense strand of the SARM1 RNAi agent has a delivery moiety conjugated to a nucleotide of the sense strand. The delivery moiety can facilitate the entry of RNAi agent into the cells. In some embodiments, the delivery moiety is α-tocopherol, palmitic acid, or another moiety in Table 4. In some embodiments, the delivery moiety is a known delivery moiety for delivering RNAi agent into a cell. Placement of a delivery moiety on the RNAi agent needs to overcome potential inefficient loading of AGO2 (Argonaute-2), or other hindrance of the RNA-induced silencing complex (RISC) complex activity. In some embodiments, the SARM1 RNAi agent is not a lipid conjugate. In some embodiments, the SARM1 RNAi agent has no delivery moiety.

In some embodiments, the delivery moiety is conjugated to the 5′ or 3′ end of the sense stand via a linker. In some embodiments, the linker is selected from Linker 1, Linker 2, Linker 3, or Linker 4 of Table 5. Other suitable linkers are known in the art. Exemplary linker—delivery moiety pairs are shown in Table 6. In some embodiments, the SARM1 RNAi agent has a linker—delivery moiety pair of Table 6.

In some embodiments, the delivery moiety is conjugated to a nucleotide of the sense strand. In that case, the delivery moiety is a modified nucleotide (e.g., a 2′-O—C₁₂₋₁₆ alkyl modified nucleotide) located in the sense strand. In some embodiments, the modified nucleotide is 2′-O-hexadecyl uridine, 2′-O-hexadecyl cytidine, 2′-O-hexadecyl guanine, or 2′-O-hexadecyl adenosine (Table 4).

TABLE 4
Delivery Moieties

| Delivery Moiety | Structure |
|---|---|
| 1 | 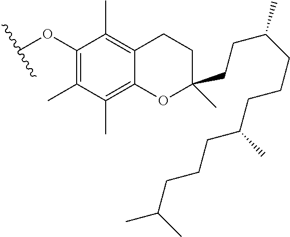 [SMILES: *Oc1c(C)c(C)c2c(c1C)CC[C@@](C)(CC[C@H](C)CCC[C@H](C)CCCC(C)C)O2] |

α-Tocopherol

| | |
|---|---|
| 2 | 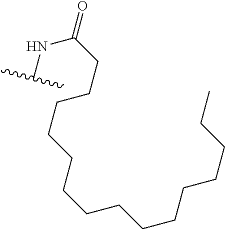 [SMILES: *NC(=O)CCCCCCCCCCCCCCC] |

Palmitic Acid

| | |
|---|---|
| 3 | 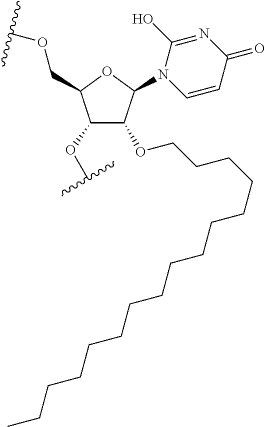 [SMILES: *OC[C@H]1O[C@@H](n2ccc(=O)nc2O)[C@H](OCCCCCCCCCCCCCCCC)[C@@H]1O*] |

Uhd (2′-O-hexadecyl uridine)

| | |
|---|---|
| 4 | 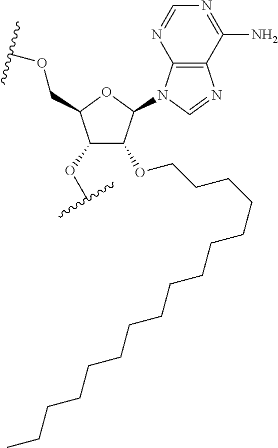 [SMILES: *OC[C@H]1O[C@@H](n2cnc3c(N)ncnc32)[C@H](OCCCCCCCCCCCCCCCC)[C@@H]1O*] |
| | Ahd (2′-O-hexadecyl adenosine) |
| 5 | 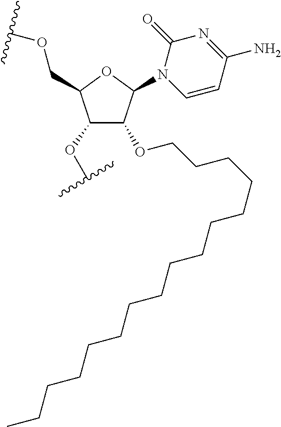 [SMILES: *OC[C@H]1O[C@@H](n2ccc(N)nc2=O)[C@H](OCCCCCCCCCCCCCCCC)[C@@H]1O*] |

Chd (2′-O-hexadecyl cytidine)

| | |
|---|---|
| 6 | 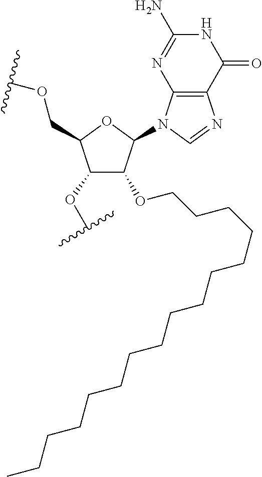 [SMILES: *OC[C@H]1O[C@@H](n2cnc3c(=O)[nH]c(N)nc32)[C@H](OCCCCCCCCCCCCCCCC)[C@@H]1O*] |

Ghd (2′-O-hexadecyl guanine)

| | |
|---|---|
| 7 | 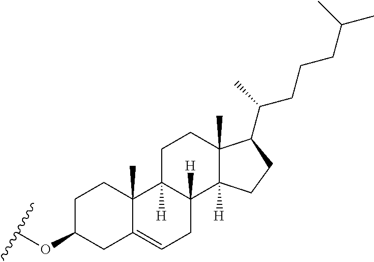 |

Cholesterol

| | |
|---|---|
| 8 | 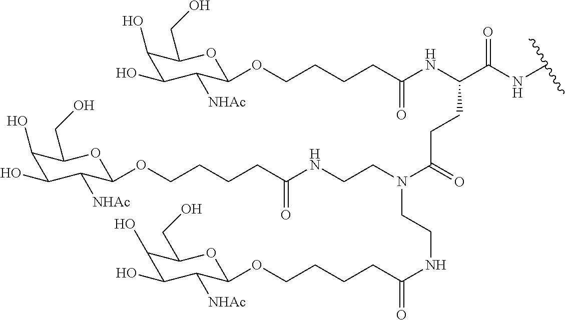 [SMILES: *NC(=O)[C@H](CCC(=O)N(CCNC(=O)CCCCOC1OC(CO)C(O)C(O)C1NC(C)=O)CCNC(=O)CCCCOC1OC(CO)C(O)C(O)C1NC(C)=O)NC(=O)CCCCOC1OC(CO)C(O)C(O)C1NC(C)=O] |
| | GalNAc |

TABLE 5
Linkers

| Linker | Structure |
|---|---|
| 1 | 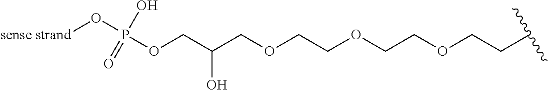 |
| | Teg (tetraethylene glycol) linker |
| 2 | 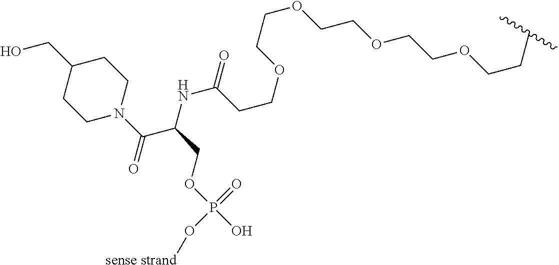 |
| | Piperidinol-PEG linker |
| 3 | 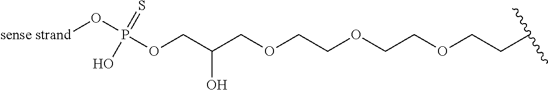 |
| | Teg (tetraethylene glycol) linker PS |
| 4 | 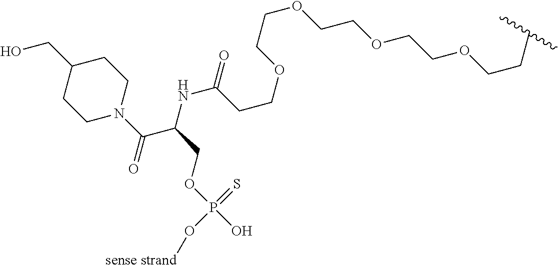 |
| | Piperidinol-PEG linker PS |
| 5 | 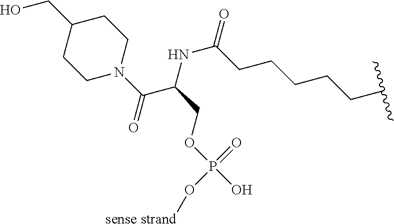 |
| | Piperidinol linker |

TABLE 6
Linker Delivery Moiety Pairs (LDP)

| LDP | Linker | Delivery Moiety |
|---|---|---|
| 1 | 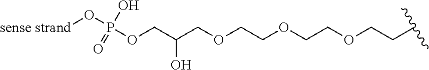 Teg (tetraethylene glycol) linker | 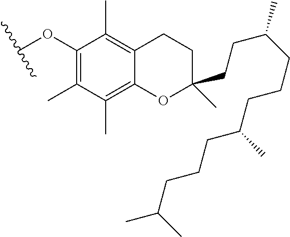 [SMILES: *Oc1c(C)c(C)c2c(c1C)CC[C@@](C)(CC[C@H](C)CCC[C@H](C)CCCC(C)C)O2] |
| | | a-Tocopherol |
| 2 | 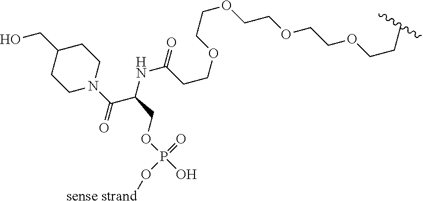 Piperidinol-PEG linker | 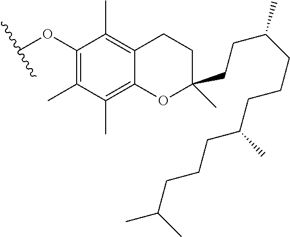 [SMILES: *Oc1c(C)c(C)c2c(c1C)CC[C@@](C)(CC[C@H](C)CCC[C@H](C)CCCC(C)C)O2] |
| | | a-Tocopherol |
| 3 | 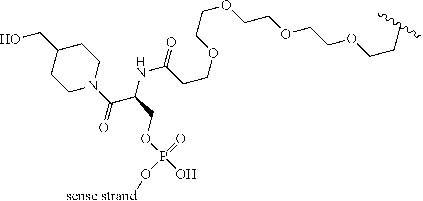 Piperidinol-PEG linker | 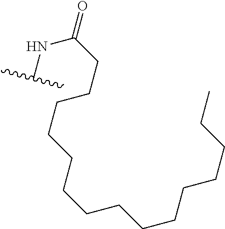 [SMILES: *NC(=O)CCCCCCCCCCCCCCC] |
| | | Palmitic Acid |
| 4 | None | 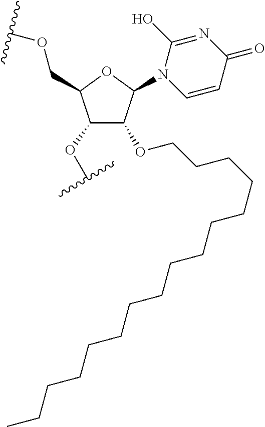 [SMILES: *OC[C@H]1O[C@@H](n2ccc(=O)nc2O)[C@H](OCCCCCCCCCCCCCCCC)[C@@H]1O*] |
| | | Uhd |
| 5 | None | 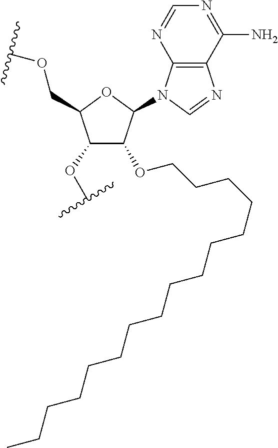 [SMILES: *OC[C@H]1O[C@@H](n2cnc3c(N)ncnc32)[C@H](OCCCCCCCCCCCCCCCC)[C@@H]1O*] |
| | | Ahd |
| 6 | None | 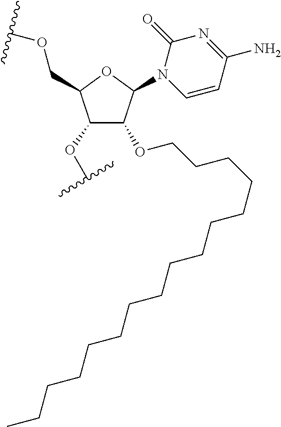 [SMILES: *OC[C@H]1O[C@@H](n2ccc(N)nc2=O)[C@H](OCCCCCCCCCCCCCCCC)[C@@H]1O*] |
| | | Chd |
| 7 | None | 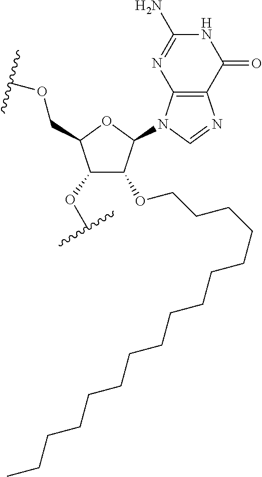 [SMILES: *OC[C@H]1O[C@@H](n2cnc3c(=O)[nH]c(N)nc32)[C@H](OCCCCCCCCCCCCCCCC)[C@@H]1O*] |
| | | Ghd |
| 8 | 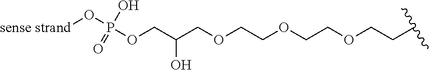 Teg (tetraethylene glycol) linker | 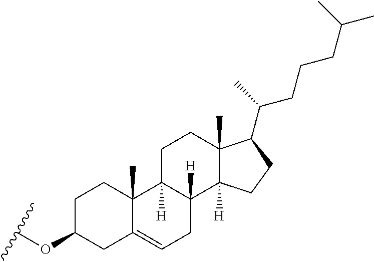 |
| | | Cholesterol |
| 9 | 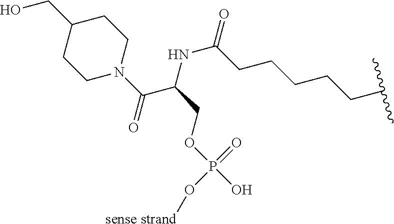 | 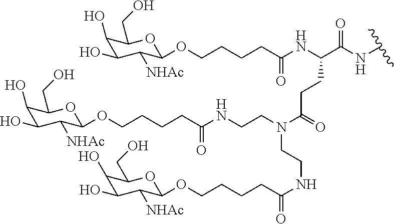 [SMILES: *NC(=O)[C@H](CCC(=O)N(CCNC(=O)CCCCOC1OC(CO)C(O)C(O)C1NC(C)=O)CCNC(=O)CCCCOC1OC(CO)C(O)C(O)C1NC(C)=O)NC(=O)CCCCOC1OC(CO)C(O)C(O)C1NC(C)=O] |
| | Piperidinol linker | GalNAc |

In a further aspect, provided herein are SARM1 RNAi agents of Formula (I): R-L-D, wherein R is a double stranded RNA (dsRNA) having a sense stand and an antisense strand, wherein the sense strand and the antisense strand form a duplex; wherein D is a delivery means (e.g., a delivery moiety) for delivering the dsRNA into a cell; and wherein L is a linking means (e.g., a linker) for linking the dsRNA to the delivery means, or optionally absent, wherein the sense strand and the antisense strand comprise a pair of nucleic acid sequences selected from the group consisting of:
(a) the sense strand comprises a first nucleic acid sequence of SEQ ID NO: 1, and the antisense strand comprises a second nucleic acid sequence of SEQ ID NO: 2;
(b) the sense strand comprises a first nucleic acid sequence of SEQ ID NO: 3, and the antisense strand comprises a second nucleic acid sequence of SEQ ID NO: 4;
(c) the sense strand comprises a first nucleic acid sequence of SEQ ID NO: 5, and the antisense strand comprises a second nucleic acid sequence of SEQ ID NO: 6;
(d) the sense strand comprises a first nucleic acid sequence of SEQ ID NO: 7, and the antisense strand comprises a second nucleic acid sequence of SEQ ID NO: 8;
(e) the sense strand comprises a first nucleic acid sequence of SEQ ID NO: 9, and the antisense strand comprises a second nucleic acid sequence of SEQ ID NO: 10; and
(f) the sense strand comprises a first nucleic acid sequence of SEQ ID NO: 11, and the antisense strand comprises a second nucleic acid sequence of SEQ ID NO: 12,
wherein optionally one or more nucleotides of the sense strand and the antisense strand are independently modified nucleotides, and wherein optionally one or more internucleotide linkages of the sense strand and the antisense strand are modified internucleotide linkages.

In some embodiments, provided herein are SARM1 RNAi agents of Formula (I): R-L-D, wherein R is a double stranded RNA (dsRNA) having a sense stand and an antisense strand, wherein the sense strand and the antisense strand form a duplex; wherein D is a delivery means (e.g., a delivery moiety) for delivering the dsRNA into a cell; and wherein L is a linking means (e.g., a linker) for linking the dsRNA to the delivery means, or optionally absent, wherein the sense strand and the antisense strand have a pair of nucleic acid sequences selected from the group consisting of:
(a) the sense strand has (e.g., consists of, or consists essentially of) a first nucleic acid sequence of SEQ ID NO: 1, and the antisense strand has (e.g., consists of, or consists essentially of) a second nucleic acid sequence of SEQ ID NO: 2;
(b) the sense strand has (e.g., consists of, or consists essentially of) a first nucleic acid sequence of SEQ ID NO: 3, and the antisense strand has (e.g., consists of, or consists essentially of) a second nucleic acid sequence of SEQ ID NO: 4;
(c) the sense strand has (e.g., consists of, or consists essentially of) a first nucleic acid sequence of SEQ ID NO: 5, and the antisense strand has (e.g., consists of, or consists essentially of) a second nucleic acid sequence of SEQ ID NO: 6;
(d) the sense strand has (e.g., consists of, or consists essentially of) a first nucleic acid sequence of SEQ ID NO: 7, and the antisense strand has (e.g., consists of, or consists essentially of) a second nucleic acid sequence of SEQ ID NO: 8;
(e) the sense strand has (e.g., consists of, or consists essentially of) a first nucleic acid sequence of SEQ ID NO: 9, and the antisense strand has (e.g., consists of, or consists essentially of) a second nucleic acid sequence of SEQ ID NO: 10; and
(f) the sense strand has (e.g., consists of, or consists essentially of) a first nucleic acid sequence of SEQ ID NO: 11, and the antisense strand has (e.g., consists of, or consists essentially of) a second nucleic acid sequence of SEQ ID NO: 12,
wherein optionally one or more nucleotides of the sense strand and the antisense strand are independently modified nucleotides, and wherein optionally one or more internucleotide linkages of the sense strand and the antisense strand are modified internucleotide linkages.

In some embodiments, provided herein are SARM1 RNAi agents of Formula (I): R-L-D, wherein R is a double stranded RNA (dsRNA) having a sense stand and an antisense strand, wherein the sense strand and the antisense strand form a duplex; wherein D is a delivery means (e.g., a delivery moiety) for delivering the dsRNA into a cell; and wherein L is a linking means (e.g., a linker) for linking the dsRNA to the delivery means, or optionally absent, wherein the sense strand and the antisense strand comprise a pair of nucleic acid sequences selected from the group consisting of:
(a) the sense strand comprises a first nucleic acid sequence of SEQ ID NO: 13, and the antisense strand comprises a second nucleic acid sequence of SEQ ID NO: 14;
(b) the sense strand comprises a first nucleic acid sequence of SEQ ID NO: 15, and the antisense strand comprises a second nucleic acid sequence of SEQ ID NO: 16;
(c) the sense strand comprises a first nucleic acid sequence of SEQ ID NO: 17, and the antisense strand comprises a second nucleic acid sequence of SEQ ID NO: 18;
(d) the sense strand comprises a first nucleic acid sequence of SEQ ID NO: 19, and the antisense strand comprises a second nucleic acid sequence of SEQ ID NO: 20;
(e) the sense strand comprises a first nucleic acid sequence of SEQ ID NO: 21, and the antisense strand comprises a second nucleic acid sequence of SEQ ID NO: 22;
(f) the sense strand comprises a first nucleic acid sequence of SEQ ID NO: 23, and the antisense strand comprises a second nucleic acid sequence of SEQ ID NO: 24;
(g) the sense strand comprises a first nucleic acid sequence of SEQ ID NO: 15, and the antisense strand comprises a second nucleic acid sequence of SEQ ID NO: 27;
(h) the sense strand comprises a first nucleic acid sequence of SEQ ID NO: 28, and the antisense strand comprises a second nucleic acid sequence of SEQ ID NO: 29;
(i) the sense strand comprises a first nucleic acid sequence of SEQ ID NO: 28, and the antisense strand comprises a second nucleic acid sequence of SEQ ID NO: 30;
(j) the sense strand comprises a first nucleic acid sequence of SEQ ID NO: 28, and the antisense strand comprises a second nucleic acid sequence of SEQ ID NO: 31;
(k) the sense strand comprises a first nucleic acid sequence of SEQ ID NO: 28, and the antisense strand comprises a second nucleic acid sequence of SEQ ID NO: 32;
(l) the sense strand comprises a first nucleic acid sequence of SEQ ID NO: 13, and the antisense strand comprises a second nucleic acid sequence of SEQ ID NO: 33;
(m) the sense strand comprises a first nucleic acid sequence of SEQ ID NO: 34, and the antisense strand comprises a second nucleic acid sequence of SEQ ID NO: 35;
(n) the sense strand comprises a first nucleic acid sequence of SEQ ID NO: 34, and the antisense strand comprises a second nucleic acid sequence of SEQ ID NO: 36;
(o) the sense strand comprises a first nucleic acid sequence of SEQ ID NO: 34, and the antisense strand comprises a second nucleic acid sequence of SEQ ID NO: 37;
(p) the sense strand comprises a first nucleic acid sequence of SEQ ID NO: 34, and the antisense strand comprises a second nucleic acid sequence of SEQ ID NO: 38;
(q) the sense strand comprises a first nucleic acid sequence of SEQ ID NO: 21, and the antisense strand comprises a second nucleic acid sequence of SEQ ID NO: 39;
(r) the sense strand comprises a first nucleic acid sequence of SEQ ID NO: 40, and the antisense strand comprises a second nucleic acid sequence of SEQ ID NO: 41;
(s) the sense strand comprises a first nucleic acid sequence of SEQ ID NO: 40, and the antisense strand comprises a second nucleic acid sequence of SEQ ID NO: 42;
(t) the sense strand comprises a first nucleic acid sequence of SEQ ID NO: 40, and the antisense strand comprises a second nucleic acid sequence of SEQ ID NO: 43;
(u) the sense strand comprises a first nucleic acid sequence of SEQ ID NO: 40, and the antisense strand comprises a second nucleic acid sequence of SEQ ID NO: 44;
(v) the sense strand comprises a first nucleic acid sequence of SEQ ID NO: 17, and the antisense strand comprises a second nucleic acid sequence of SEQ ID NO: 45;
(w) the sense strand comprises a first nucleic acid sequence of SEQ ID NO: 19, and the antisense strand comprises a second nucleic acid sequence of SEQ ID NO: 46; and
(x) the sense strand comprises a first nucleic acid sequence of SEQ ID NO: 23, and the antisense strand comprises a second nucleic acid sequence of SEQ ID NO: 47.

In some embodiments, provided herein are SARM1 RNAi agents of Formula (I): R-L-D, wherein R is a double stranded RNA (dsRNA) having a sense stand and an antisense strand, wherein the sense strand and the antisense strand form a duplex; wherein D is a delivery means (e.g., a delivery moiety) for delivering the dsRNA into a cell; and wherein L is a linking means (e.g., a linker) for linking the dsRNA to the delivery means, or optionally absent, wherein the sense strand and the antisense strand have a pair of nucleic acid sequences selected from the group consisting of:
(a) the sense strand has (e.g., consists of, or consists essentially of) a first nucleic acid sequence of SEQ ID NO: 13, and the antisense strand has (e.g., consists of, or consists essentially of) a second nucleic acid sequence of SEQ ID NO: 14;
(b) the sense strand has (e.g., consists of, or consists essentially of) a first nucleic acid sequence of SEQ ID NO: 15, and the antisense strand has (e.g., consists of, or consists essentially of) a second nucleic acid sequence of SEQ ID NO: 16;
(c) the sense strand has (e.g., consists of, or consists essentially of) a first nucleic acid sequence of SEQ ID NO: 17, and the antisense strand has (e.g., consists of, or consists essentially of) a second nucleic acid sequence of SEQ ID NO: 18;
(d) the sense strand has (e.g., consists of, or consists essentially of) a first nucleic acid sequence of SEQ ID NO: 19, and the antisense strand has (e.g., consists of, or consists essentially of) a second nucleic acid sequence of SEQ ID NO: 20;
(e) the sense strand has (e.g., consists of, or consists essentially of) a first nucleic acid sequence of SEQ ID NO: 21, and the antisense strand has (e.g., consists of, or consists essentially of) a second nucleic acid sequence of SEQ ID NO: 22;
(f) the sense strand has (e.g., consists of, or consists essentially of) a first nucleic acid sequence of SEQ ID NO: 23, and the antisense strand has (e.g., consists of, or consists essentially of) a second nucleic acid sequence of SEQ ID NO: 24;
(g) the sense strand has (e.g., consists of, or consists essentially of) a first nucleic acid sequence of SEQ ID NO: 15, and the antisense strand has (e.g., consists of, or consists essentially of) a second nucleic acid sequence of SEQ ID NO: 27;
(h) the sense strand has (e.g., consists of, or consists essentially of) a first nucleic acid sequence of SEQ ID NO: 28, and the antisense strand has (e.g., consists of, or consists essentially of) a second nucleic acid sequence of SEQ ID NO: 29;
(i) the sense strand has (e.g., consists of, or consists essentially of) a first nucleic acid sequence of SEQ ID NO: 28, and the antisense strand has (e.g., consists of, or consists essentially of) a second nucleic acid sequence of SEQ ID NO: 30;
(j) the sense strand has (e.g., consists of, or consists essentially of) a first nucleic acid sequence of SEQ ID NO: 28, and the antisense strand has (e.g., consists of, or consists essentially of) a second nucleic acid sequence of SEQ ID NO: 31;
(k) the sense strand has (e.g., consists of, or consists essentially of) a first nucleic acid sequence of SEQ ID NO: 28, and the antisense strand has (e.g., consists of, or consists essentially of) a second nucleic acid sequence of SEQ ID NO: 32;
(l) the sense strand has (e.g., consists of, or consists essentially of) a first nucleic acid sequence of SEQ ID NO: 13, and the antisense strand has (e.g., consists of, or consists essentially of) a second nucleic acid sequence of SEQ ID NO: 33;
(m) the sense strand has (e.g., consists of, or consists essentially of) a first nucleic acid sequence of SEQ ID NO: 34, and the antisense strand has (e.g., consists of, or consists essentially of) a second nucleic acid sequence of SEQ ID NO: 35;
(n) the sense strand has (e.g., consists of, or consists essentially of) a first nucleic acid sequence of SEQ ID NO: 34, and the antisense strand has (e.g., consists of, or consists essentially of) a second nucleic acid sequence of SEQ ID NO: 36;
(o) the sense strand has (e.g., consists of, or consists essentially of) a first nucleic acid sequence of SEQ ID NO: 34, and the antisense strand has (e.g., consists of, or consists essentially of) a second nucleic acid sequence of SEQ ID NO: 37;
(p) the sense strand has (e.g., consists of, or consists essentially of) a first nucleic acid sequence of SEQ ID NO: 34, and the antisense strand has (e.g., consists of, or consists essentially of) a second nucleic acid sequence of SEQ ID NO: 38;
(q) the sense strand has (e.g., consists of, or consists essentially of) a first nucleic acid sequence of SEQ ID NO: 21, and the antisense strand has (e.g., consists of, or consists essentially of) a second nucleic acid sequence of SEQ ID NO: 39;
(r) the sense strand has (e.g., consists of, or consists essentially of) a first nucleic acid sequence of SEQ ID NO: 40, and the antisense strand has (e.g., consists of, or consists essentially of) a second nucleic acid sequence of SEQ ID NO: 41;
(s) the sense strand has (e.g., consists of, or consists essentially of) a first nucleic acid sequence of SEQ ID NO: 40, and the antisense strand has (e.g., consists of, or consists essentially of) a second nucleic acid sequence of SEQ ID NO: 42;
(t) the sense strand has (e.g., consists of, or consists essentially of) a first nucleic acid sequence of SEQ ID NO: 40, and the antisense strand has (e.g., consists of, or consists essentially of) a second nucleic acid sequence of SEQ ID NO: 43;
(u) the sense strand has (e.g., consists of, or consists essentially of) a first nucleic acid sequence of SEQ ID NO: 40, and the antisense strand has (e.g., consists of, or consists essentially of) a second nucleic acid sequence of SEQ ID NO: 44;
(v) the sense strand has (e.g., consists of, or consists essentially of) a first nucleic acid sequence of SEQ ID NO: 17, and the antisense strand has (e.g., consists of, or consists essentially of) a second nucleic acid sequence of SEQ ID NO: 45;
(w) the sense strand has (e.g., consists of, or consists essentially of) a first nucleic acid sequence of SEQ ID NO: 19, and the antisense strand has (e.g., consists of, or consists essentially of) a second nucleic acid sequence of SEQ ID NO: 46; and
(x) the sense strand has (e.g., consists of, or consists essentially of) a first nucleic acid sequence of SEQ ID NO: 23, and the antisense strand has (e.g., consists of, or consists essentially of) a second nucleic acid sequence of SEQ ID NO: 47.

In some embodiments, the delivery means is conjugated to the 3′ end of the sense strand. In some embodiments, the delivery means is conjugated to the 5′ end of the sense strand. In some embodiments, the delivery means is conjugated to a nucleotide of the sense strand. In some embodiments, the delivery means is palmitic acid, α-tocopherol, or another moiety in Table 4. In some embodiments, the linking means is selected from the group consisting of Linker 1, Linker 2, Linker 3, and Linker 4 of Table 5.

The sense strand and antisense strand of SARM1 RNAi agent can be synthesized using any nucleic acid polymerization methods known in the art, for example, solid-phase synthesis by employing phosphoramidite chemistry methodology (e.g., Current Protocols in Nucleic Acid Chemistry, Beaucage, S. L. et al. (Edrs.), John Wiley & Sons, Inc., New York, NY, USA), H-phosphonate, phosphortriester chemistry, or enzymatic synthesis. Automated commercial synthesizers can be used, for example, MerMade™ 12 from LGC Biosearch Technologies, or other synthesizers from BioAutomation or Applied Biosystems. Phosphorothioate linkages can be introduced using a sulfurizing reagent such as phenylacetyl disulfide or DDTT (((dimethylaminomethylidene) amino)-3H-1,2,4-dithiazaoline-3-thione). It is well known to use similar techniques and commercially available modified amidites and controlled-pore glass (CPG) products to synthesize modified oligonucleotides or conjugated oligonucleotides.

Purification methods can be used to exclude the unwanted impurities from the final oligonucleotide product. Commonly used purification techniques for single stranded oligonucleotides include reverse-phase ion pair high performance liquid chromatography (RP-IP-HPLC), capillary gel electrophoresis (CGE), anion exchange HPLC (AX-HPLC), and size exclusion chromatography (SEC). After purification, oligonucleotides can be analyzed by mass spectrometry and quantified by spectrophotometry at a wavelength of 260 nm. The sense strand and antisense strand can then be annealed to form a duplex.

In another aspect, provided herein are pharmaceutical compositions comprising a SARM1 RNAi agent described herein and a pharmaceutically acceptable carrier. Also provided herein are pharmaceutical compositions comprising a means for reducing SARM1 expression in a cell and a pharmaceutically acceptable carrier. Such pharmaceutical compositions can also comprise one or more pharmaceutically acceptable excipient, diluent, or carrier. Pharmaceutical compositions can be prepared by methods well known in the art (e.g., Remington: The Science and Practice of Pharmacy, 23rd edition (2020), A. Loyd et al., Academic Press).

In a further aspect, provided herein are methods of reducing SARM1 expression in a cell (e.g., a neuron), such methods can include introducing a SARM1 RNAi agent described herein into the cell; and incubating the cell for a time sufficient for degradation of SARM1 mRNA, thereby reducing SARM1 expression in the cell. The SARM1 RNAi agent can be introduced into the cell (e.g., a neuron) using a method known in the art, e.g., transfection, electroporation, microinjection, or uptake by the cell via natural transport mechanisms.

In another aspect, provided herein are methods of reducing SARM1 expression in a patient in need thereof, and such method comprises administering to the patient an effective amount of a SARM1 RNAi agent or a pharmaceutical composition described herein.

In another aspect, provided herein are methods of reducing axon degeneration in a patient in need thereof, and such method comprises administering to the patient an effective amount of a SARM1 RNAi agent or a pharmaceutical composition described herein.

In another aspect, provided herein are methods of treating a SARM1-mediated neurological disease in a patient in need thereof, and such method comprises administering to the patient an effective amount of the SARM1 RNAi agent or a pharmaceutical composition described herein. Exemplary SARM1-mediated neurological disease includes, but are not limited to, amyotrophic lateral sclerosis (ALS, or Lou Gehrig's disease), Alzheimer's disease, Parkinson's disease, multiple sclerosis (MS), Huntington's disease (HD), senile dementia, Pick's disease, Gaucher's disease, Hurler syndrome, progressive multifocal leukoencephalopathy, Alexander's disease, congenital hypomyelination, encephalomyelitis, acute disseminated encephalomyelitis, central pontine myelinolysis, osmotic hyponatremia, Tay-Sachs disease, motor neuron disease, ataxia, spinal muscular atrophy (SMA), Niemann-Pick disease, acute hemorrhagic leukoencephalitis, trigeminal neuralgia, Bell's palsy, cerebral ischemia, multiple system atrophy, Pelizaeus Merzbacher disease, periventricular leukomalacia, a hereditary ataxia, noise-induced hearing loss, congenital hearing loss, age-related hearing loss, Creutzfeldt-Jakob disease, transmissible spongiform encephalopathy, Lewy Body Dementia, frontotemporal dementia, tauopathy, synucleinopathy, amyloidosis, diabetic neuropathy, globoid cell leukodystrophy (Krabbe's disease), Bassen-Komzweig syndrome, transverse myelitis, motor neuron disease, spinocerebellar ataxia, pre-eclampsia, hereditary spastic paraplegias, spastic paraparesis, familial spastic paraplegia, French settlement disease, Strumpell-Lorrain disease, non-alcoholic steatohepatitis (NASH), adrenomyeloneuropathy, progressive supra nuclear palsy (PSP), Friedrich's ataxia, spinal cord injury, acute optic neuropathy (AON), a genetic or idiopathic retinal condition, Leber congenital amaurosis (LCA), Leber hereditary optic neuropathy (LHON), primary open-angle glaucoma (POAG), acute angle-closure glaucoma (AACG), autosomal dominant optic atrophy, retinal ganglion degeneration, retinitis pigmentosa, an outer retinal neuropathy, optic nerve neuritis, optic nerve degeneration associated with multiple sclerosis, Kjer's optic neuropathy, ischemic optic neuropathy, chemotherapy-induced peripheral neuropathy, neuromyelitis optica, Charcot Marie Tooth disease, deficiency in vitamin B12, deficiency in folic acid (vitamin B9), isolated vitamin E deficiency syndrome, non-arteritic anterior ischemic optic neuropathy, exposure to ethambutol, exposure to cyanide, traumatic brain injury (TBI), spinal cord injury, traumatic axonal injury or chronic traumatic encephalopathy (CTE). In some embodiments, the SARM1-mediated neurological disease is amyotrophic lateral sclerosis, multiple sclerosis, chemotherapy-induced peripheral neuropathy (CIPN), diabetic peripheral neuropathy (DPN), tauopathy, or Charcot Marie Tooth disease. In some embodiments, the SARM1-mediated neurological disease is amyotrophic lateral sclerosis.

The SARM1 RNAi agent can be administered to the patient intrathecally, intracerebroventricularly, or via intracisternal magna injection. In some embodiments, the SARM1 RNAi agent is administered to the patient intrathecally, e.g., via a catheter or direct injection into the intrathecal space.

RNAi dosage regimens may be adjusted to provide the optimum desired response (e.g., a therapeutic response). For example, a single bolus may be administered, several divided doses may be administered over time, or the dose may be proportionally reduced or increased as indicated by the exigencies of the therapeutic situation.

Dosage values may vary with the type and severity of the condition to be alleviated. It is further understood that for any particular subject, specific dosage regimens should be adjusted over time according to the individual need and the professional judgment of the person administering or supervising the administration of the compositions.

In another aspect, provided herein are SARM1 RNAi agents or pharmaceutical compositions comprising a SARM1 RNAi agent for use in reducing SARM1 expression. Also provided herein are SARM1 RNAi agents or pharmaceutical compositions comprising a SARM1 RNAi agent for use in reducing axon degeneration. Also provided herein are SARM1 RNAi agents or the pharmaceutical composition comprising a SARM1 RNAi agent for use in a therapy. Also provided herein are SARM1 RNAi agents or pharmaceutical compositions comprising a SARM1 RNAi agent for use in the treatment of a SARM1-mediated neurological disease. Also provided herein are uses of SARM1 RNAi agents in the manufacture of a medicament for reducing axon degeneration. Also provided herein are uses of SARM1 RNAi agents in the manufacture of a medicament for the treatment of a SARM1-mediated neurological disease.

As used herein, the terms “a,” “an,” “the,” and similar terms used in the context of the present disclosure (especially in the context of the claims) are to be construed to cover both the singular and plural unless otherwise indicated herein or clearly contradicted by the context.

As used herein, the term “alkyl” means saturated linear or branched-chain monovalent hydrocarbon radical, containing the indicated number of carbon atoms. For example, “C₁-C₂₀ alkyl” means a radical having 1-20 carbon atoms in a linear or branched arrangement.

As used herein, “antisense strand” means a single-stranded oligonucleotide that is complementary to a region of a target sequence. Likewise, and as used herein, “sense strand” means a single-stranded oligonucleotide that is complementary to a region of an antisense strand.

As used herein, “complementary” means a structural relationship between two nucleotides (e.g., on two opposing nucleic acids or on opposing regions of a single nucleic acid strand, e.g., a hairpin) that permits the two nucleotides to form base pairs with one another. For example, a purine nucleotide of one nucleic acid that is complementary to a pyrimidine nucleotide of an opposing nucleic acid may base pair together by forming hydrogen bonds with one another. Complementary nucleotides can base pair in the Watson-Crick manner or in any other manner that allows for the formation of stable duplexes. Likewise, two nucleic acids may have regions of multiple nucleotides that are complementary with each other to form regions of complementarity, as described herein.

As used herein, a “delivery moiety” refers to a moiety that facilitates the entry of an oligonucleotide or RNAi agent into a cell. The delivery moiety can be lipid, cholesterol, vitamin E, carbohydrate, amino sugar, polypeptide, or combination thereof.

As used herein, “duplex,” in reference to nucleic acids or oligonucleotides, means a structure formed through complementary base pairing of two antiparallel sequences of nucleotides (i.e., in opposite directions), whether formed by two covalently separate nucleic acid strands or by a single, folded strand (e.g., via a hairpin).

An “effective amount” refers to an amount necessary (for periods of time and for the means of administration) to achieve the desired therapeutic result. An effective amount of a RNAi agent may vary according to factors such as the disease state, age, sex, and weight of the individual, and the ability of the RNAi agent to elicit a desired response in the individual. An effective amount is also one in which any toxic or detrimental effects of the RNAi agent are outweighed by the therapeutically beneficial effects.

The term “knockdown” or “expression knockdown” refers to reduced mRNA or protein expression of a gene after treatment of a reagent, e.g., a RNAi agent.

As used herein, “modified internucleotide linkage” means an internucleotide linkage having one or more chemical modifications when compared with a reference internucleotide linkage having a phosphodiester bond. A modified internucleotide linkage can be a non-naturally occurring linkage. In some embodiments, the modified internucleotide linkage is phosphorothioate linkage.

As used herein, “modified nucleotide” refers to a nucleotide having one or more chemical modifications when compared with a corresponding reference nucleotide selected from: adenine ribonucleotide, guanine ribonucleotide, cytosine ribonucleotide, uracil ribonucleotide, adenine deoxyribonucleotide, guanine deoxyribonucleotide, cytosine deoxyribonucleotide, and thymidine deoxyribonucleotide. A modified nucleotide can have, for example, one or more chemical modification in its sugar, nucleobase, and/or phosphate group. Additionally, or alternatively, a modified nucleotide can have one or more chemical moieties conjugated to a corresponding reference nucleotide. In some embodiments, the modified nucleotide is a 2′-fluoro modified nucleotide, 2′-O-methyl modified nucleotide, or 2′-O-alkyl modified nucleotide. In some embodiments, the modified nucleotide has a phosphate analog, e.g., 5′-vinylphosphonate. In some embodiments, the modified nucleotide has an abasic moiety or inverted abasic moiety, e.g., a moiety shown in Table 3.

As used herein, the term “SARM1-mediated neurological disease” refers to a neurological disease, disorder, or injury mediated by SARM1 and/or by axonal degeneration.

As used herein, “nucleotide” means an organic compound having a nucleoside (a nucleobase, e.g., adenine, cytosine, guanine, thymine, or uracil, and a pentose sugar, e.g., ribose or 2′-deoxyribose) linked to a phosphate group. A “nucleotide” can serve as a monomeric unit of nucleic acid polymers such as deoxyribonucleic acid (DNA) and ribonucleic acid (RNA).

As used herein, “oligonucleotide” means a polymer of linked nucleotides, each of which can be modified or unmodified. An oligonucleotide is typically less than about 100 nucleotides in length.

As used herein, “overhang” means the unpaired nucleotide or nucleotides that protrude from the duplex structure of a double stranded oligonucleotide. An overhang may include one or more unpaired nucleotides extending from a duplex region at the 5′ terminus or 3′ terminus of a double stranded oligonucleotide. The overhang can be a 3′ or 5′ overhang on the antisense strand or sense strand of a double stranded oligonucleotide.

The term “patient”, as used herein, refers to a human patient.

As used herein, “phosphate analog” means a chemical moiety that mimics the electrostatic and/or steric properties of a phosphate group. In some embodiments, a phosphate analog is positioned at the 5′ terminal nucleotide of an oligonucleotide in place of a 5′-phosphate, which is often susceptible to enzymatic removal. A 5′ phosphate analog can include a phosphatase-resistant linkage. Examples of phosphate analogs include 5′ methylene phosphonate (5′-MP) and 5′-(E)-vinylphosphonate (5′-VP). In some embodiments, the phosphate analog is 5′-VP.

The term “% sequence identity” or “percentage sequence identity” with respect to a reference nucleic acid sequence is defined as the percentage of nucleotides, nucleosides, or nucleobases in a candidate sequence that are identical with the nucleotides, nucleosides, or nucleobases in the reference nucleic acid sequence, after optimally aligning the sequences and introducing gaps or overhangs, if necessary, to achieve the maximum percent sequence identity. Alignment for purposes of determining percent nucleic acid sequence identity can be achieved in various ways that are within the skill in the art, for instance, using publicly available computer software programs, for example, those described in Current Protocols in Molecular Biology (Ausubel et al., eds., 1987, Supp. 30, section 7.7.18, Table 7.7.1), and including BLAST, BLAST-2, ALIGN, Megalign (DNASTAR), Clustal W2.0 or Clustal X2.0 software. Those skilled in the art can determine appropriate parameters for measuring alignment, including any algorithms needed to achieve maximal alignment over the full length of the sequences being compared. Percentage of “sequence identity” can be determined by comparing two optimally aligned sequences over a comparison window, where the fragment of the nucleic acid sequence in the comparison window may comprise additions or deletions (e.g., gaps or overhangs) as compared to the reference sequence (which does not comprise additions or deletions) for optimal alignment of the two sequences. The percentage can be calculated by determining the number of positions at which the identical nucleotide, nucleoside, or nucleobase occurs in both sequences to yield the number of matched positions, dividing the number of matched positions by the total number of positions in the window of comparison, and multiplying the result by 100 to yield the percentage of sequence identity. The output is the percent identity of the subject sequence with respect to the query sequence.

As used herein, “RNAi,” “RNAi agent,” “iRNA,” “iRNA agent,” and “RNA interference agent” means an agent that mediates sequence-specific degradation of a target mRNA by RNA interference, e.g., via RNA-induced silencing complex (RISC) pathway. In some embodiments, the RNAi agent has a sense strand and an antisense strand, and the sense strand and the antisense strand form a duplex (e.g., a double stranded RNA). In some embodiments, the sense strand has a delivery moiety conjugated to the 5′ or 3′ end of the sense strand or a nucleotide of the sense strand.

As used herein, “strand” refers to a single, contiguous sequence of nucleotides linked together through internucleotide linkages (e.g., phosphodiester linkages or phosphorothioate linkages). A strand can have two free ends (e.g., a 5′ end and a 3′ end).

As used herein, “SARM1” (sterile alpha and TIR motif containing 1, also known as SARM; HsTIR; SAMD2; hSARM1; MyD88-5) refers to a human SARM1 mRNA transcript or a human SARM1 protein. The nucleotide sequence of human SARM1 mRNA can be found at NM_015077.4:

| (SEQ ID NO: 25) | |
|---|---|
| 1 | ATCTCCCAGC TCAGCCGAGC CCGTGCCCAG GCCACGCTTT GTTCCAGCCG CCGCCTCCTC |
| 61 | TACCCTACGG CGTCCGGAGC CATCCCTCGC CTGCTCGCTC TCTCCTTTCG CCCACTCCCT |
| 121 | GCATCTGGGC CTGCATCACC TTTGCCAACC GCTCCCCCGA TCCTGCCGAC ACTCCTCCCC |
| 181 | CAAACTTCTG ACCGGCACCC TTGCCTGGTA CCCTTCTCTC CATTCCTCCC CCTCCATCTT |
| 241 | CTTTCCCCGA CCCCTCTCGG GTCCCTCTTT TCCCAAAACC CGGGTCTCTC CGCGTGGCCC |
| 301 | CGCCTCCAGG CCGGGGATGT CCCCCGCGGC CCCGCGCCCA TGGTCCTGAC GCTGCTTCTC |
| 361 | TCCGCCTACA AGCTGTGTCG CTTCTTCGCC ATGTCGGGCC CACGGCCGGG CGCCGAGCGG |
| 421 | CTGGCGGTGC CTGGGCCAGA TGGGGGCGGT GGCACGGGCC CATGGTGGGC TGCGGGTGGC |
| 481 | CGCGGGCCCC GCGAAGTGTC GCCGGGGGCA GGCACCGAGG TGCAGGACGC CCTGGAGCGC |
| 541 | GCGCTGCCGG AGCTGCAGCA GGCCTTGTCC GCGCTGAAGC AGGCGGGCGG CGCGCGGGCC |
| 601 | GTGGGCGCCG GCCTGGCCGA GGTCTTCCAA CTGGTGGAGG AGGCCTGGCT GCTGCCGGCC |
| 661 | GTGGGCCGCG AGGTAGCCCA GGGTCTGTGC GACGCCATCC GCCTCGATGG CGGCCTCGAC |
| 721 | CTGCTGTTGC GGCTGCTGCA GGCGCCGGAG TTGGAGACGC GTGTGCAGGC CGCGCGCCTG |
| 781 | CTGGAGCAGA TCCTGGTGGC TGAGAACCGA GACCGCGTGG CGCGCATTGG GCTGGGCGTG |
| 841 | ATCCTGAACC TGGCGAAGGA ACGCGAACCC GTAGAGCTGG CGCGGAGCGT GGCAGGCATC |
| 901 | TTGGAGCACA TGTTCAAGCA TTCGGAGGAG ACATGCCAGA GGCTGGTGGC GGCCGGCGGC |
| 961 | CTGGACGCGG TGCTGTATTG GTGCCGCCGC ACGGACCCCG CGCTGCTGCG CCACTGCGCG |
| 1021 | CTGGCGCTGG GCAACTGCGC GCTGCACGGG GGCCAGGCGG TGCAGCGACG CATGGTAGAG |
| 1081 | AAGCGCGCAG CCGAGTGGCT CTTCCCGCTC GCCTTCTCCA AGGAGGACGA GCTGCTTCGG |
| 1141 | CTGCACGCCT GCCTCGCAGT AGCGGTGTTG GCGACTAACA AGGAGGTGGA GCGCGAGGTG |
| 1201 | GAGCGCTCGG GCACGCTGGC GCTCGTGGAG CCGCTTGTGG CCTCGCTGGA CCCTGGCCGC |
| 1261 | TTCGCCCGCT GTCTGGTGGA CGCCAGCGAC ACAAGCCAGG GCCGCGGGCC CGACGACCTG |
| 1321 | CAGCGCCTCG TGCCGTTGCT CGACTCTAAC CGCTTGGAGG CGCAGTGCAT CGGGGCTTTC |
| 1381 | TACCTCTGCG CCGAGGCTGC CATCAAGAGC CTGCAAGGCA AGACCAAGGT GTTCAGCGAC |
| 1441 | ATCGGCGCCA TCCAGAGCCT GAAACGCCTG GTTTCCTACT CTACCAATGG CACTAAGTCG |
| 1501 | GCGCTGGCCA AGCGCGCGCT GCGCCTGCTG GGCGAGGAGG TGCCACGGCC CATCCTGCCC |
| 1561 | TCCGTGCCCA GCTGGAAGGA GGCCGAGGTT CAGACGTGGC TGCAGCAGAT CGGTTTCTCC |
| 1621 | AAGTACTGCG AGAGCTTCCG GGAGCAGCAG GTGGATGGCG ACCTGCTTCT GCGGCTCACG |
| 1681 | GAGGAGGAAC TCCAGACCGA CCTGGGCATG AAATCGGGCA TCACCCGCAA GAGGTTCTTT |
| 1741 | AGGGAGCTCA CGGAGCTCAA GACCTTCGCC AACTATTCTA CGTGCGACCG CAGCAACCTG |
| 1801 | GCGGACTGGC TGGGCAGCCT GGACCCGCGC TTCCGCCAGT ACACCTACGG CCTGGTCAGC |
| 1861 | TGCGGCCTGG ACCGCTCCCT GCTGCACCGC GTGTCTGAGC AGCAGCTGCT GGAAGACTGC |
| 1921 | GGCATCCACC TGGGCGTGCA CCGCGCCCGC ATCCTCACGG CGGCCAGAGA AATGCTACAC |
| 1981 | TCCCCGCTGC CCTGTACTGG TGGCAAACCC AGTGGGGACA CTCCAGATGT CTTCATCAGC |
| 2041 | TACCGCCGGA ACTCAGGTTC CCAGCTGGCC AGTCTCCTGA AGGTGCACCT GCAGCTGCAT |
| 2101 | GGCTTCAGTG TCTTCATTGA TGTGGAGAAG CTGGAAGCAG GCAAGTTCGA GGACAAACTC |
| 2161 | ATCCAGAGTG TCATGGGTGC CCGCAACTTT GTGTTGGTGC TATCACCTGG AGCACTGGAC |
| 2221 | AAGTGCATGC AAGACCATGA CTGCAAGGAT TGGGTGCATA AGGAGATTGT GACTGCTTTA |
| 2281 | AGCTGCGGCA AGAACATTGT GCCCATCATT GATGGCTTCG AGTGGCCTGA GCCCCAGGTC |
| 2341 | CTGCCTGAGG ACATGCAGGC TGTGCTTACT TTCAACGGTA TCAAGTGGTC CCACGAATAC |
| 2401 | CAGGAGGCCA CCATTGAGAA GATCATCCGC TTCCTGCAGG GCCGCTCCTC CCGGGACTCA |
| 2461 | TCTGCAGGCT CTGACACCAG TTTGGAGGGT GCTGCACCCA TGGGTCCAAC CTAACCAGTC |
| 2521 | CCCAGTTCCC CAGCCCTGCT GTGACTTCCA TTTCCATCGT CCTTTCTGAA GGAACAGCTC |
| 2581 | CTGAAACCAG TCTCCCTGGG CTGAGACAAC CTGGGCTCTT CTTAGGAAAT GGCTCTCCCT |
| 2641 | CCCCCTGTCC CCCACCCTCA TGGCCCACCT CCAACCCACT TTCCTCAGTA TCTGGAGAGG |
| 2701 | GAAGGGAAGT CAGGCTTGGG CACGGGAGGT TAGAACTCCC CCAGGCCCTG CCATTGGGTT |
| 2761 | GTCTGTCTCC GTCATGGGGA GGGTCCCTGC TCAGTTCTGG AGACACTGGA GTTGGGGTGG |
| 2821 | GGGTGGTTCT GCATTCCCTT CTCCTGCTGA TAGCAGTCAG CTTGAGGAGG ATGACGGAAG |
| 2881 | GCAGCCTCAG ACAGGAATTA AGGCAATGCC CAGGCGGGCC TGGGCACTGT ATTCTGAGCA |
| 2941 | AGGGCCTGGG CCCAGGAGCC AGCCAGGGAT GAGTGCCATC ATGGCTCTCC ACTCAGACTG |
| 3001 | TGCCTGGCCC CTGCACTTAC AACTTCCTGC CGCTCTGTGG CCTTGCCCTG TAATCACTCA |
| 3061 | GTGCCCTTAG CTAGCCTGAC TAAGTCCCAG ATCCCCTACA GCTTCCTTCG GTGTGGTATC |
| 3121 | TTTTGCCACA TCCAGGGCGA GGGTTGAGGC AAACCAGCCC TCCCTCTGAC TTCCTTGTCA |
| 3181 | CTGCAGCCAG CTTTGCTGCA CTTGCTGGTG CACAGGAGCC TCCTGTTTGG GCCTGGGTCT |
| 3241 | GGGCATGGGG AGGCCGTGCC TCAAAGCCCA CCCTACCCCA TGCCTTGGTG CTGTGCCTCA |
| 3301 | GGCTCCTTCC TGGTCTGGCC CAGCTGGCTT CCCCAGCCCC TCAGCCATCC AGGGCTACCC |
| 3361 | ACTGCTTACT CAGGGACCAG GCAGCCCCCA TGGCAGTAAA AGCAGCCTAG ACAGAACCTG |
| 3421 | CAGCTCTGTG GAAAGAGGCA AAGTCCTGAA AAGGCAAAGG GTTGTCACTT AGGGCAGCTT |
| 3481 | CTCCAACTTT AACATGCATC CAAGTCACCT GGGAATGTTG TTAAAATCAG GAGATCTGGG |
| 3541 | GTGGGGCCTA GGACTCTGCA TTTCTTACAG ATTCCCAGGT GAGCTGATGC TGGTGGTTAA |
| 3601 | GGGTAGCAAA TCTCTAAAGC ACGAAGCCCT CACAAATCTT TGCCATTTCC CAAACACTCC |
| 3661 | GCTCCATGGT CTCCAGTCAT CAGAGCAACT CTACCTGGTA TTATCATCCC CATTTTACAG |
| 3721 | ATAATGACAC TGAGGCTCAG AAAGGTTGAG GATAAGCCCA CTTTCCTGTC ATTAGTGGCA |
| 3781 | GCCCCAGATC CAGACCTAGG CCTCCTGGCA CCCAGTCCAC TGGCAGTGGA ATTGCTTTCC |
| 3841 | TGAGAATCAT TCTGAGGCTG GGCTATTGCT TCTCCCTTGC TTCAAAGAAT CTAGCAGCGG |
| 3901 | GGGATAGGAT TTTGCAACAA AAAGCTGACC CAGAGGCCAT ACAGAGCAGG AATATCCCAT |
| 3961 | TGCCCCCTCC TCCACTGGGT TCAGAGGGTA AGAAAGCACC CTCCAATAAA CCCAGGCTCC |
| 4021 | AGGCCGTGGG GGCTGCTGAA GGCTCTTTCC CCGCAAGGGC CAGGTGTTGA CACCTTAAAG |
| 4081 | CTGGCTGCGC CCCCAGCCCC ACTCTTGGCT GTGCTGGCCA GGTGACTCCT AGTTCTTGGC |
| 4141 | CACATCATCA GAAAGTCAAA GGTCTCACTC CAGGTTTGGG GCTCCTTCCT TCCACTCCCC |
| 4201 | TCCCTGCCAG AGTCTGTCTT GGCCAGTGCC AGCCTCGATG CTTTGGTTTT GACCCCACCT |
| 4261 | GATCCTCCTT TCCTCATGCA GCACAAGTGC TCACCGGGGC CAGAGCCAGG GCATGGATAT |
| 4321 | GACAAGCAGG GCAGCCTGGA CACTGCCCTC ACAGGACAGC GCCAATAACA ATACAGTGTC |
| 4381 | TGAGTATCTC CAGGGGATGA TTTCTGGCTC TTTGTCTCCA ATCAGTCCCA CTCCCTCCTG |
| 4441 | AGGTCCCCAA GGGCAGTATT CAGAGAGGTT TCCTGCGTTT TATTTCTATT TGGTATACCC |
| 4501 | TCCACTGTTG TCCACTGCCC TGTGTGGCCT TCTGGTTGAC CTCTGCCCGA TCTTCTGTCT |
| 4561 | CTCTGAGGGA ATCAGAGTCC AGCATCCAGC CCCAGCTGGA ACAGCTGAAG TCACAAGCCT |
| 4621 | CCTCTAAGCC AAGGCCAGTG TGTTCAGAGG TGACTGCCAC CCATACTAGG ACAAACACAG |
| 4681 | CTCAGATCAC CAGGTCAAGC ACCTAGGCCT GGCTTCTCCT GAGACAGAGG ACTCAGAAGT |
| 4741 | GGCCTTTCCT CCAAAGCCTG CTCAGACACA GGTCTGTAGG GCCAGGGTGT TCTGCTTGGC |
| 4801 | TGGGCTGCAG CTGCTACCCC TCGGTTGGGG CTGAGTCAGC CAGATCCTCC CCCTACTTCT |
| 4861 | CCCCAAGGGC CAAGAACTGC TCAGGGACAT TAAAGGTCAA AAGTCCAGCC ACACTCATTC |
| 4921 | ATCCTTTCCC CAGGCCCATG AAGAGAGGCA TCTCATTGTA GAATGTATGA GGAAGTGGGA |
| 4981 | AGTATCTCAG AGAATCAGCT AAGTTTCCTA ACTTGTCCAT CCAAATGTGA TCACCACGAT |
| 5041 | TCAACAATTT GGGGCATTGC TGATCTAGCC GTTCCTAGTG GGGCTTGCTC AAGGTTGCAC |
| 5101 | AGCGAGTCAG TAGAAGCCCT GGCTGGCCCC ACTTGGTACC AATCCACCAG GCAGCTCAGG |
| 5161 | GCTCCTGCCC AGCCCAGCAG CTTCTGTTGT CTAACGTATG GCAGGCAGAC TGGGAGCAGG |
| 5221 | AAAACAGAGG GCCCCAAAGC CCAAGGCACC AGAAGGTTTG TTTCAGTTTG CTGAAGCTGA |
| 5281 | TTTGTAATGA TTGGCACTCT TCAGCCAGGG GAGTGGGTAG GCCATAGCCA AGGATCGATT |
| 5341 | CCCCAACCAC AGCAAAGGCA ACACTCTTCC TCCAGAGATC ACCAAGCCCC TCTTACCTCC |
| 5401 | CTCCCTCCTT CCCAAGGCTG GCACTAACCA GGTACCACAT TCATTGTTAA GGAATGGCTG |
| 5461 | ATGACTGCTA CACGTGTTGG GAACCTGGTT GGGGCTGTGC AGTTTGGGCT GGAAGGAGAG |
| 5521 | ATGCCAGCCC TCGTGCTGCC TCTGGTCCCT GAAGTGTCAC CTCTCTCAGG ACCTCTCCTC |
| 5581 | TGGCCTGTGG GGTTATAAGT GATGGATAGC AGAAAGGGAG AACTGACTCC TGTCCCAAAT |
| 5641 | AGCTCCTCTG CCACCTGTCC TGCAGTGGGC CTGTGTGGGT TATGATTCTA GATCCTAGAC |
| 5701 | AGAGGCTGGG TCAGCTGTGG ATGGGGTGGT GCCTTGGTCT CTCTTGACTA CCTCGTCCAA |
| 5761 | AGAGAGCACT GCCCTTAGAC AAGAGTTGCT TGTCCTGCTG TGGGCTGGGC TTCCAGCTGC |
| 5821 | AGACCTCCAG TTGCTTGGTG TTCACTTTGC TCCTCTTGCC CTCTGTCTTC TGGTCCAGGC |
| 5881 | AGATCAGGGG CTCTGGGGAA ACTGCTGGAA CTCGAGGTGA GGATCAGCCT TTTCCAGCAT |
| 5941 | CCTGTGAGAG ACCAGAGAGA GAGTTTGGAT TTCATGTGGG GAACCCTCAA GGCCTGTCTG |
| 6001 | GAGAAGTGAC ACAGGATTTA CTGGGGTGGG CTGGTCCAGG TAGCTCTCCT GAACCTCCTC |
| 6061 | CTTCCCCAAG CTGAGAAGCT GAGAGCTGGA GGACAATATC CAGGGACATG GCTCTGGAAA |
| 6121 | ATAACTTTTT TTTTTTTAAG AGACAGGGTC TTGCTCTGTT GTCCAGGCTG GAGGGCAGTG |
| 6181 | ACATAATCAT AGCTCACTGT ACCCTTGAAC TCCTGGGCTC AAGTGATCCT CCTGCCTCAG |
| 6241 | CCTCCTTAGT AGCTGGGACT ACCAGTGCAT ACCACCATGC CTGGGTGATT TTTTAAATTT |
| 6301 | TTTATACAGA CAAGGTCTTG CTATGTTGCC CAGGCTGATC TTGAATTCCC GGGCTCAAGT |
| 6361 | GGTCCTCCTG CCTCAGCCTC CCACAGGATC GGGATTACAG GCAAGAGCCT CCACGCCCGG |
| 6421 | CCATGAAATA TAATTCTTAA TATCATACAG GAAAAAGTCA GCGGGTCAAG CTAGCCTGTG |
| 6481 | GCCCAGCCAC AACTAGCTGA CAAAGCTTCC TGGCCTTCCC TTTAACACAG TTCTGCTGCC |
| 6541 | ATAGTTCCAT CTATAAAATG GGAATGGAGG GAAATAGGGG AACTGGGAGA GAGAACACAG |
| 6601 | CCTTGCCAAG CAGCAATGTT AGCCTGATCC TTCCTCCACC TAGCTCGCCA TCTCGCCCTT |
| 6661 | GGAAAATGGC TCCTGGAGGA TTAGGCAGCC ATCTGCAAGG AGAGGGGCAA CCTGGGACAA |
| 6721 | GACACCCAGA GGGTAAGGAT TCCAGGAATG AAGCTGCCAT TTCTGGTTGG GAGGAGAAGA |
| 6781 | GGAAACTTTT AAGAGAAAGG GCTCCATTAT GAGCATGGGT TCAGGGCCCT GCATTACCCA |
| 6841 | ATCAGAACAG CCGGGATGAG CAGGAGGCCA GCTCCCAGGA GGAAGGGGAA CCCCTTCATA |
| 6901 | AAGTTCAGAG TGGCTGGGTA GAGTGAGTTG AAGATGCCGG AGGCCGTCAG CATGGCCAGG |
| 6961 | CTATTCACAC AGGCCACAGC AGAAAAGAGA GCACCTGTGA AGAAATAAAT ACCATACTCT |
| 7021 | GGAGTCCGAA AGGGCCATAT TCCAACTCTG GCACCACCAC CTCACAGCTG TGTGACCGGG |
| 7081 | AGTAGTCACT TAACCTATGT CTCCCCTTCC TCACCAGTAA ATCCTGCTAC ATCATGTACT |
| 7141 | GTGACAAGGA TTCAGTAAGG TCATATGTGG ACAGTAGCTG GCACAGAGGG GCTACTAAAC |
| 7201 | AAATGGCTGC TATTAAATCC ACATTAAAAG TACATGTGAT CTGACAGAAC CCAGCACATA |
| 7261 | AAAGAAAAAA AAAGTACATG TGATATTGTC TGATGAAAGC TTGATGGAAA TGGCTTTTTT |
| 7321 | CTGGTTTATC CTCTTTGGAA TCATCTCCTG TTTGGGATTA ACTGCTGGTC TGATCAGTTC |
| 7381 | CAATATTCAT AGCGGTGTCA CCACTGAATA GCTTCTTATC CTTTGGGTTC CTGTTCCTCC |
| 7441 | TTCTGCTAAA TAAGGATAAT ACCTATTTCC TAGATTGTGA GCAACATTAA GTTCACATGG |
| 7501 | AAATCACCCA TCACTGGGCC TGGTCCCCTG GAAGTAGCTA GTTAGTAAGG GCTGTTCTTT |
| 7561 | TCTCCTGTTT CTCTTGACAT CTCTGGGCAC AGAGAAAGTG CTGGGAAAAA AAGTTTAGGT |
| 7621 | GAATGAATGA AGACACATGG ATTCTGGGGA CACCAGAACC CACAGTGGGC TCTGTATGGC |
| 7681 | ACCAGAGTCT CTGTCATCAT CAGATCCTCA TTCCAGGACA GATGGAAAAA GATGAATGTT |
| 7741 | TCCAGACTGG GGCATAAAGA CCCAGAGGCT GGAGAAGCTG TTCTTTATAG ATATACCAGG |
| 7801 | AGAACCCACA GTTTACAAAA TGTGCAACAA CCCAACAGAA GTTGAGATTA AATTCTGTCA |
| 7861 | CATCTAGAGG GGTCTGTGAT GTCATCAAAA GCAAACCACC CACATCACAG ATGAAGAAAC |
| 7921 | AGGCCTGTGG CAGGGCTCGG ACTAAAACCC AGATCCTGAG ACCAGCTGCT TTTAAACACA |
| 7981 | GACGTAGGTT TGCATCCTAG CTCCACCATT TACTGAGTAA CCTTGGGTGA GCCAATGTAA |
| 8041 | CCCCCTGGGT CTCTGTTTCT TTATCTGTCA ACTGTGGAAA ATGAAACCCA TGTCACAAGG |
| 8101 | TTGTTCACTT CTGGGCTTGT ACACGCTGAC CCCAGAGAAA CAGGGAACTC TGGCATCACC |
| 8161 | ACACCCATCT TACAGACGGA AAAGCTGAGG TCTGCAGAGA GTAAATCCTC TGCTCTGGTT |
| 8221 | ATCTAGAAAG AACATAATTG TGCTCTGCTG ACTGCAAATC CCAACTCTGC GGTTTGAAAA |
| 8281 | TCCAAGGTGG CATGATCCTC TGCCCATTGT GGGCAATTTC ACAGAAATGT GTTTGTTTTG |
| 8341 | GCCACTTACT TCTCCAGGGT GAGAGGGGGG AAGGCAAGCT GTTCCCCCAG CCATGGCTGC |
| 8401 | CCATCAGCCC GTTTCGGGCA GCACTGGACA TGAGGAACCA GACACAGGTG GGTTCTGACA |
| 8461 | CTCACCCTGC TCTGTCTCTC TCACCAGCTT GGAGAGTTTA GCCCGGATGA CAGGTGTGAT |
| 8521 | GACTAATGAC AGGAAAAGCA ACCCATATCC TGTGGAGAAA CAAACACTCA TCAGGAAAAT |
| 8581 | GGGGCTGGGG AGAGGGGCGT CCAAGGGAAA GGCAGCAGAG CTCCTATCCA TACCCCACGT |
| 8641 | GGGGCTTAGG TTAGACCCAG GAAGAACTTC CTTGATGGTG AGGGTGGGAA GACAGTAGTC |
| 8701 | AAGGAGGAAT GGAGACTGCC CTTGTCTGGG CTTGGCCACC TGCTAGCTCT CATGAATGAA |
| 8761 | TGCTAATTCC CATTGATTGC TTTCTTGTCT GAACCTCTTG TGGTCACAGC AGGCATCACC |
| 8821 | CACCCACTTG GCACTTAGTA GGGATATGGC AGGGCACAGA AAACAAGCAT GGGCTTTGGA |
| 8881 | GTCAGCCCTG AGTTCAAAAC CTGATGCCAT TACATATTAT CTGTGTGGCC TGGGGTACTT |
| 8941 | ACCCTCTCTG ATCCTGACTC CCTGTATGAG GAAGATAATA AGGCCTTCAT CACAGGATGG |
| 9001 | TTCTGAGGCA TAGGAGGCTG AATAATGGTG CCCAATGGCA TCAGATTCAT AGCCCTGGAA |
| 9061 | CCTGTAAATA CTACCTTATT TGGAAAATGA GTCTATGCAG GTGTGCAGTT AAGCCTCCTG |
| 9121 | AGAGAGCAGA GTTATCCTGG ATTAGGTTGG GCCCTAAATG CCGTCACACA TATCTTTATA |
| 9181 | AGAGGAAAGC AGACGGAGAT TTGGCACCGA CAGAATTGAG AAGGCACAAA GAGGAGGAGA |
| 9241 | GTCAATGTGA GCACAGAGGC AGAGACTGGT GATGGCCGCC CCAAGCCAAG GAATGCCAGC |
| 9301 | AGCCCCAGAA GCTGGAAGAA ATGAGAAACA CGTTCTCTCC TGGAGGCTTG CAAGGGAGCA |
| 9361 | CTGCCTGCTG ACTGCTTCCA TTCAGCCCGG TGGTACTGAC TTTGGACTTC TGGCCTCCAG |
| 9421 | AACTGTGAGA GAATATGTTT CTGTTGTGTT AAGCCCCCAA GTTTGTGGTA TGTCATTACA |
| 9481 | GCAATCTCAG GGAACCAATA CATGAGGTAA AAAGGTAACA TCTATGAAGA GCATGGCATA |
| 9541 | GGGACACAGC AAATGGGAGT TCCTTTTCCC TTTGCATTCA GTTACTTACA GGCTTCCTGT |
| 9601 | TTTCTTCATA ACCATTTCTC TCCCTGTGCG ACTGCTGACT CCTCAGCAAA ACTGCAAACT |
| 9661 | CCTACAGGAC AGTGGATCCT CCAAAGAAGG TATACGATGA GGCATCCAGG GACCCTAGCA |
| 9721 | GTGTCAGGCC CCTCAAATCC CACTCTGTTG AGACCTCCCC CCGACCCAGA GCAATGACAG |
| 9781 | CATCTTTATC ATCTCTGCAT CCCCCAGGGC CATCAGCAGG AGGGAAAGGT TCCCTTCTGC |
| 9841 | TTAATTGTCA GACAAGCAGT TGAGTTAAGA AATCTGTGAT TATTGTATTG TTGACTATAC |
| 9901 | ACAGCACATT TTAGGGCTCT ATCAAAATAA ATCTGTCCCT TTAAAAAAAG TTAACTAAAG |
| 9961 | CCGGGCACGG TGGCTCATGC CTGTAATCCC AACACTTTGG GAGGCTGAGG CAGGCGGATC |
| 10021 | CTTGAGCTCA GGAGTTAGAG ACCTGGACTG GGCAAAATGG TGAGGACCCC ATCTCTATAA |
| 10081 | AAAATACAAA AATTAGCAAG GTGTGGTAAT GTGCACCAGT GGTCCCAGCT ACTAGAGAGG |
| 10141 | CCAAGGTGGG AGGATCATCT GGGCCCGGGG GATGAGGCTG CAGTGAGCCA TGATCGTGCC |
| 10201 | ACTGCACTCT AGCCTGGGTA ACAAAGCGAG ACCCTGTCTC TAAATACATC AATCAAATAA |
| 10261 | AAATTTTAAA AAGTTAA. |

| (SEQ ID NO: 26) | |
|---|---|
| 1 | MVLTLLLSAY KLCRFFAMSG PRPGAERLAV PGPDGGGGTG PWWAAGGRGP REVSPGAGTE |
| 61 | VQDALERALP ELQQALSALK QAGGARAVGA GLAEVFQLVE EAWLLPAVGR EVAQGLCDAI |
| 121 | RLDGGLDLLL RLLQAPELET RVQAARLLEQ ILVAENRDRV ARIGLGVILN LAKEREPVEL |
| 181 | ARSVAGILEH MFKHSEETCQ RLVAAGGLDA VLYWCRRTDP ALLRHCALAL GNCALHGGQA |
| 241 | VQRRMVEKRA AEWLFPLAFS KEDELLRLHA CLAVAVLATN KEVEREVERS GTLALVEPLV |
| 301 | ASLDPGRFAR CLVDASDTSQ GRGPDDLQRL VPLLDSNRLE AQCIGAFYLC AEAAIKSLQG |
| 361 | KTKVFSDIGA IQSLKRLVSY STNGTKSALA KRALRLLGEE VPRPILPSVP SWKEAEVQTW |
| 421 | LQQIGFSKYC ESFREQQVDG DLLLRLTEEE LQTDLGMKSG ITRKRFFREL TELKTFANYS |
| 481 | TCDRSNLADW LGSLDPRFRQ YTYGLVSCGL DRSLLHRVSE QQLLEDCGIH LGVHRARILT |
| 541 | AAREMLHSPL PCTGGKPSGD TPDVFISYRR NSGSQLASLL KVHLQLHGFS VFIDVEKLEA |
| 601 | GKFEDKLIQS VMGARNFVLV LSPGALDKCM QDHDCKDWVH KEIVTALSCG KNIVPIIDGE |
| 661 | EWPEPQVLPE DMQAVLTENG IKWSHEYQEA TIEKIIRFLQ GRSSRDSSAG SDTSLEGAAP |
| 721 | MGPT. |

As used herein, “subject” means a mammal, including cat, dog, mouse, rat, chimpanzee, ape, monkey, and human. Preferably the subject is a human.

As used herein, “treatment” or “treating” refers to all processes wherein there may be a slowing, controlling, delaying, or stopping of the progression of the disorders or disease disclosed herein, or ameliorating disorder or disease symptoms, but does not necessarily indicate a total elimination of all disorder or disease symptoms. Treatment includes administration of a protein or nucleic acid or vector or composition for treatment of a disease or condition in a patient, particularly in a human.

### EXAMPLES

### Example 1. Synthesis of Linker—Delivery Moiety Pairs

Certain abbreviations are defined as follows: “ACN” refers to acetonitrile; “AEX” refers to anion exchange; “C/D” refers to cleavage and deprotection; “CPG” refers to controlled pore glass; “DCM” refers to dichloromethane; “DEA” refers to diethylamine; “DIEA” refers to N,N-diisopropylethylamine; “DMAP” refers to 4-dimethylaminopyridine; “DMF” refers to dimethylformamide; “DMSO” refers to dimethyl sulfoxide; “DMTCl” refers to 4,4′-dimethoxytrityl chloride; “ES/MS” refers to electrospray mass spectrometry; “EtOAc” refers to ethyl acetate; “EtOH” refers to ethanol and ethyl alcohol; “HBTU” refers to 3-[bis(dimethylamino)methyliumyl]-3H-benzotriazol-1-oxide hexafluorophosphate; “HOBt” refers to 1-hydroxybenzotriazole; “IP-RP” refers to ion-pair reverse phase; “LCAA CPG” refers to long chain alkylamine controlled pore glass; “LC/MS” refers to liquid chromatography-mass spectrometry; “MeOH” refers to methanol and methyl alcohol; “MPA” refers to mobile phase A; “MPB” refers to mobile phase B; “MWCO” refers to molecular weight cut-off; “NMR” refers to nuclear magnetic resonance; “PBS” phosphate-buffered saline; “PEG” refers to polyethylene glycol; “PVDF” refers to polyvinylidene fluoride; “RP” refers to reverse phase; “RPM” refers to revolutions per minute; “siRNA” refers to small interfering ribonucleic acid; “TEA” refers to triethylamine; “THF” refers to tetrahydrofuran; “TLC” refers to thin line chromatography; “TMP” refers to 2,2,6,6-tetramethylpiperidine; “UPLC” refers to ultra-performance liquid chromatography; and “UV” refers to ultraviolet.

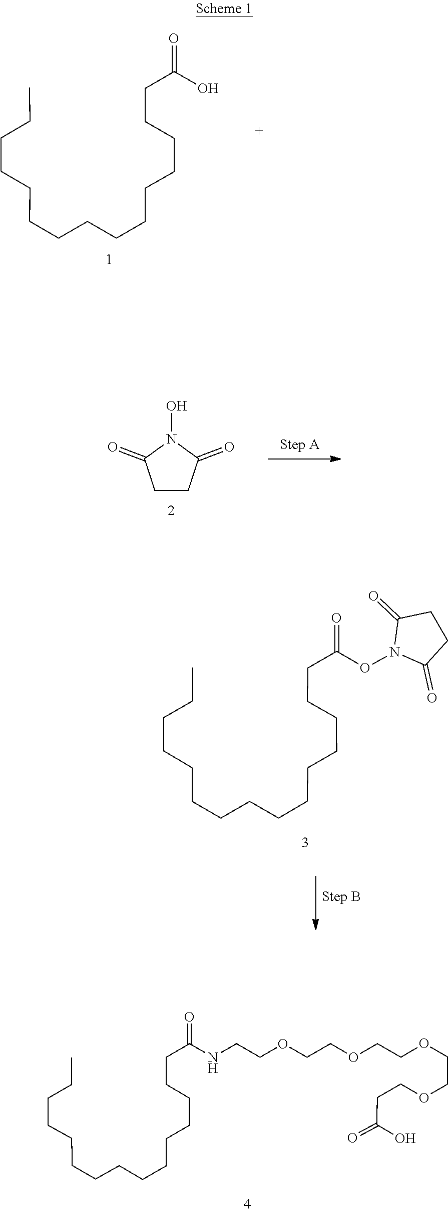 [SMILES: CCCCCCCCCCCCCCCC(=O)O ; O=C1CCC(=O)N1O ; CCCCCCCCCCCCCCCC(=O)ON1C(=O)CCC1=O ; CCCCCCCCCCCCCCCC(=O)NCCOCCOCCOCCOCCC(=O)O]

Scheme 1, step A depicts the coupling of compounds (1) and (2) using an appropriate base such as DMAP in a suitable solvent such as DCM to give compound (3). Step B shows the coupling of compound (3) with 1-amino-3,6,9,12-tetraoxapentadecan-15-oic acid in the presence of a base such as potassium carbonate and in a solvent system such as water and THF to give compound (4).

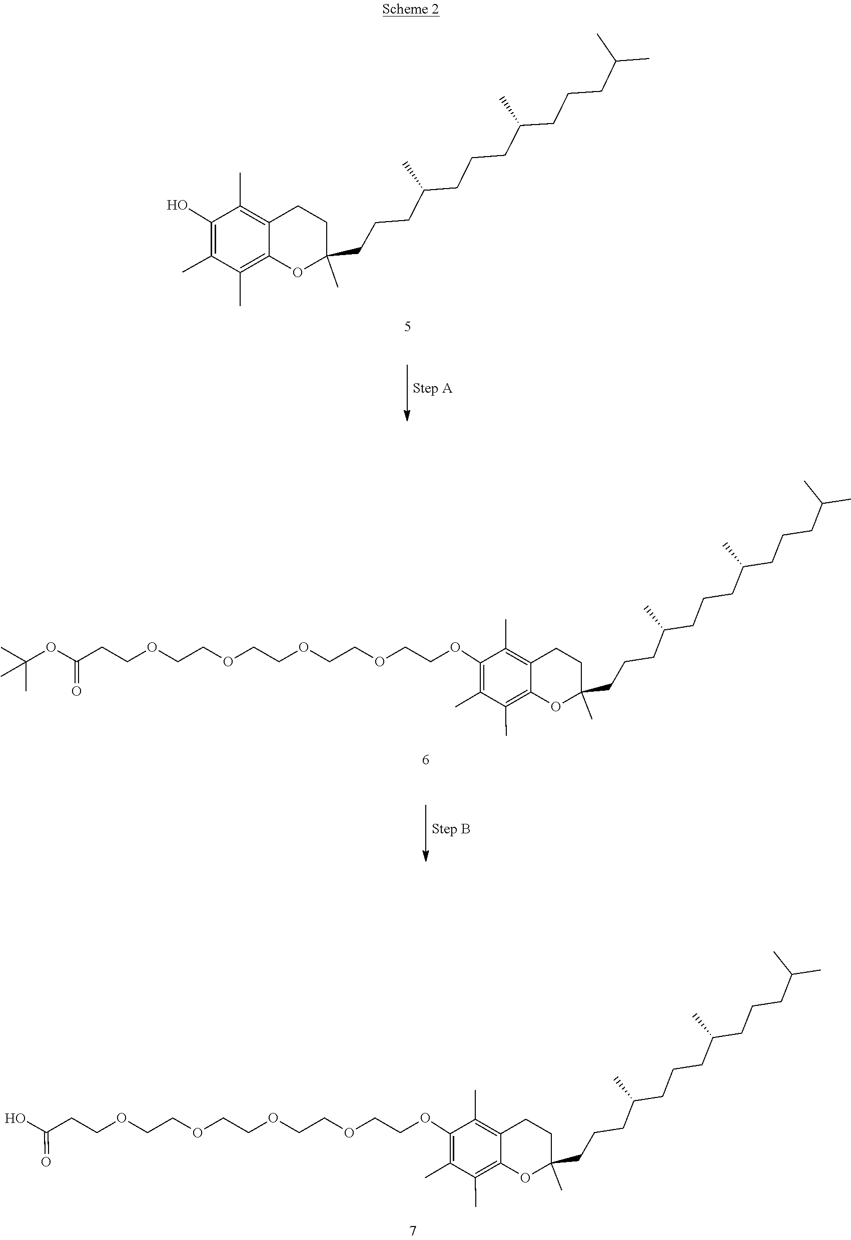 [SMILES: Cc1c(C)c2c(c(C)c1O)CC[C@@](C)(CCC[C@H](C)CCC[C@H](C)CCCC(C)C)O2 ; Cc1c(C)c2c(c(C)c1OCCOCCOCCOCCOCCC(=O)OC(C)(C)C)CC[C@@](C)(CCC[C@H](C)CCC[C@H](C)CCCC(C)C)O2 ; Cc1c(C)c2c(c(C)c1OCCOCCOCCOCCOCCC(=O)O)CC[C@@](C)(CCC[C@H](C)CCC[C@H](C)CCCC(C)C)O2]

Scheme 2, step A depicts a Mitsunobu reaction between compound (5) and tert-butyl 1-hydroxy-3,6,9,12-tetraoxapentadecan-15-oate using triphenyl phosphene and diisopropyl azodicarboxylate in a solvent such as THE to give compound (6). Step B shows the acidic deprotection of compound (6) using an acid such as HCl in a solvent such as 1,4-dioxane to give compound (7).

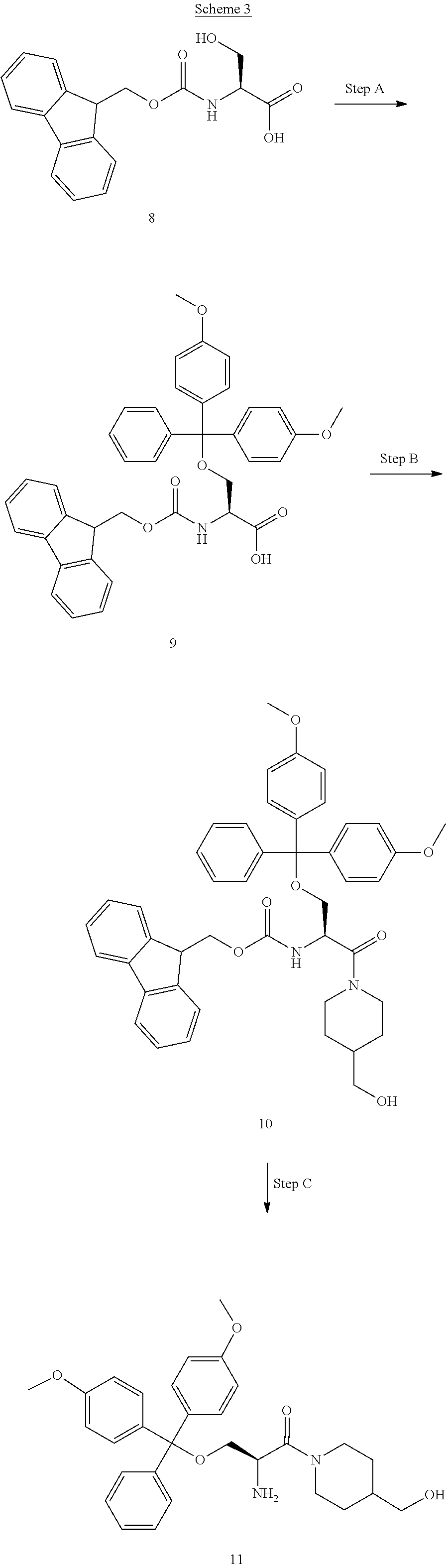

Scheme 3, step A depicts the protection of compound (8) using DMTCl with a suitable base such as DIEA in a solvent such as DCM to give compound (9). Step B shows an amide coupling between compound (9) and piperidin-4-yl methanol using HBTU and HOBt with TMP in a solvent such as DCM to give compound (10). The deprotection of compound (10) with 20% piperidine in DMF to give compound (11) is shown in step C.

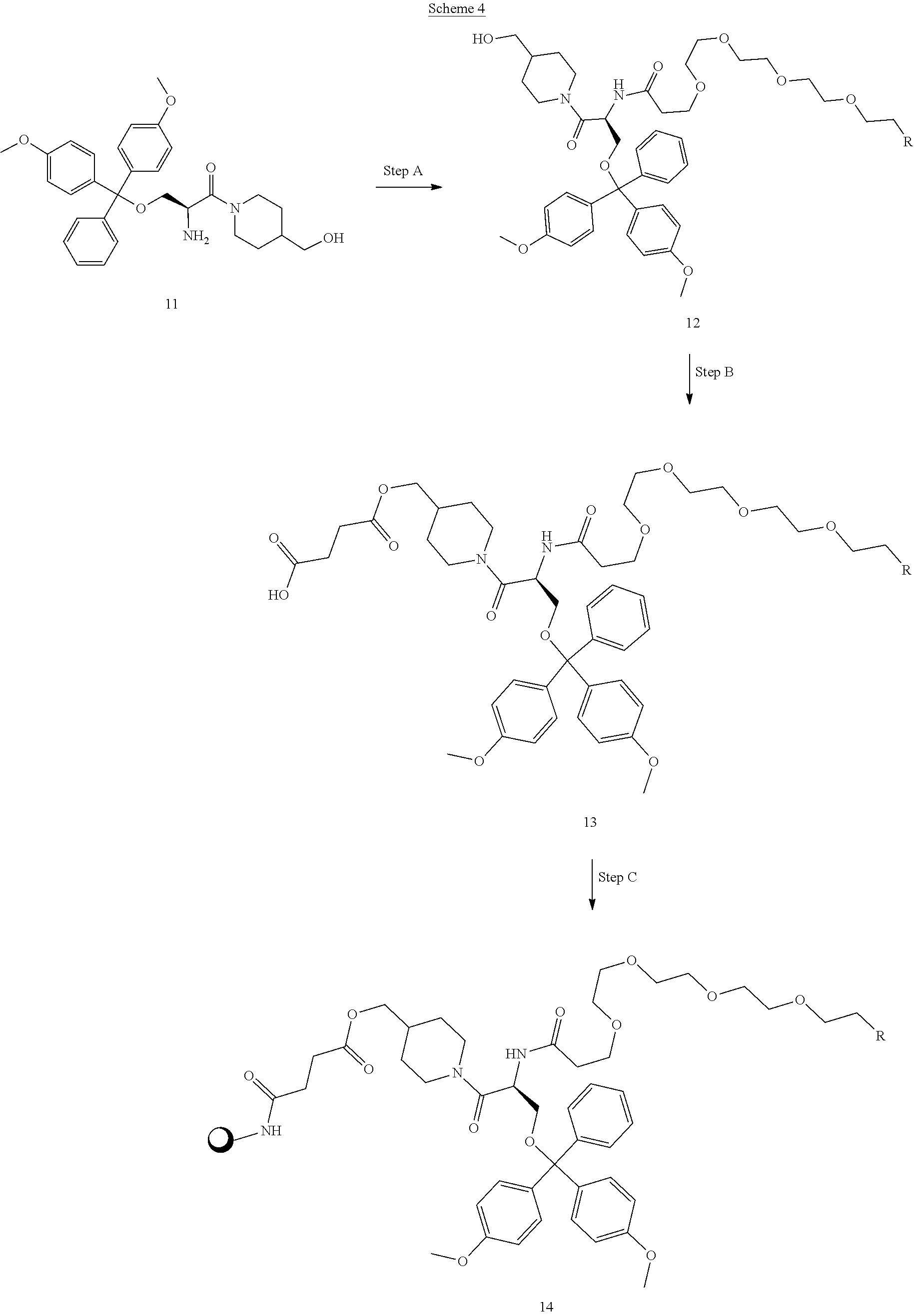

Scheme 4, step A depicts an amide coupling between compound (11) and either compound (4) or compound (7) using standard coupling reagents such as HBTU and HOBt with a base such as DIEA in a solvent such as DMF to give compound (12). One skilled in the art will recognize the variety of conditions which could be used to perform this amide coupling. Step B shows the coupling of compound (12) to succinic anhydride using a base such as TEA with catalytic DMAP in a solvent such as DCM to give compound (13). Step C shows the amide coupling of compound (13) to amino LCAA CPG using HBTU with a base such as DIEA in a solvent such as ACN followed by a multistep work up to give compound (14).

### Preparation 1

### 2,5-Dioxopyrrolidin-1-yl palmitate

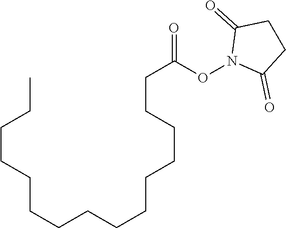 [SMILES: CCCCCCCCCCCCCCCC(=O)ON1C(=O)CCC1=O]

Added palmitic acid (2.00 g, 7.80 mmol) to a solution of 1-(3-dimethylaminopropyl)-3-ethylcarbodiimide hydrochloride (1.79 g, 9.36 mmol) and DMAP (0.19 g, 1.56 mmol) in DCM (31 mL). Stirred the mixture at ambient temperature for 5 minutes then added N-hydroxysuccinimide (0.99 g, 8.58 mmol) and stirred at ambient temperature for 18 hours. Concentrated in vacuo and purified the resulting crude material via silica gel flash chromatography eluting with a gradient of 0-80% EtOAc in hexanes to give the title compound as a white solid (2.65 g, 96%). ¹H NMR (DMSO-d₆) δ 2.81 (s, 4H), 2.66 (t, 2H), 1.62 (m, 2H), 1.25 (br s, 24H), 0.87 (t, 3H).

### Preparation 2

### 3-[2-[2-[2-[2-(Hexadecanoylamino)ethoxy]ethoxy]ethoxy]ethoxy]propanoic acid

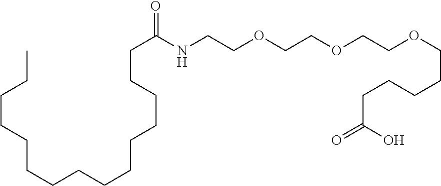 [SMILES: CCCCCCCCCCCCCCCC(=O)NCCOCCOCCOCCCCCC(=O)O]

Added 1-amino-3,6,9,12-tetraoxapentadecan-15-oic acid (0.14 g, 0.53 mmol) to a solution of potassium carbonate (0.14 g, 1.00 mmol) in THE (1 mL) and water (2 mL). Added 2,5-dioxopyrrolidin-1-yl palmitate (0.18 g, 0.51 mmol) and stirred the reaction at ambient temperature for 18 hours. Quenched the reaction with water (30 mL) and adjusted the pH to ˜3 with 1N aqueous HCl. A precipitate formed and was collected by vacuum filtration to give the title compound as a white solid (0.19 g, 74%). ES/MS m z 504 (M+H).

### Preparation 3

### tert-Butyl 3-[2-[2-[2-[2-[(2R)-2,5,7,8-tetramethyl-2-[(4R,8R)-4,8,12-trimethyltridecyl]chroman-6-yl]oxyethoxy]ethoxy]ethoxy]ethoxy]propanoate

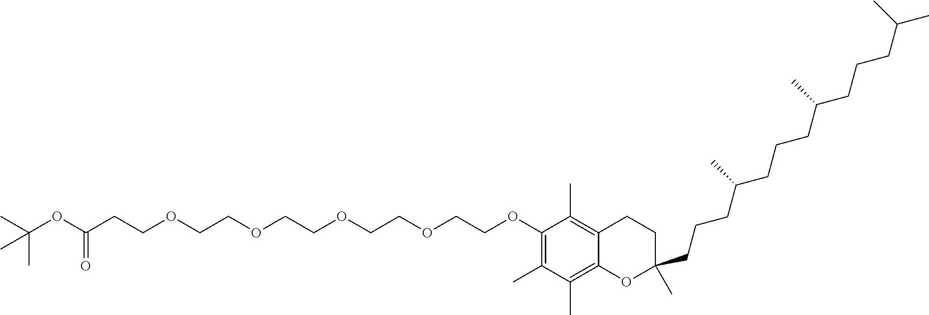 [SMILES: Cc1c(C)c2c(c(C)c1OCCOCCOCCOCCOCCC(=O)OC(C)(C)C)CC[C@@](C)(CCC[C@H](C)CCC[C@H](C)CCCC(C)C)O2]

Combined (2R)-2,5,7,8-tetramethyl-2-[(4R,8R)-4,8,12-trimethyltridecyl]chroman-6-ol (3.00 g, 6.90 mmol), tert-butyl 1-hydroxy-3,6,9,12-tetraoxapentadecan-15-oate (2.50 g, 7.60 mmol), and triphenylphosphine (2.00 g, 7.60 mmol) in THF (28.0 mL) and added diisopropyl azodicarboxylate (1.50 mL, 7.60 mmol) dropwise over 5 minutes. Heated the mixture at 60° C. for 16 hours. Cooled the mixture to ambient temperature, added silica gel, and concentrated in vacuo to give an off-white solid. Purified the mixture via silica gel flash chromatography, eluting with 0-40% EtOAc/hexanes, to give the title compound as an oil (3.33 g, 66%). ¹H NMR (CDCl₃): 3.84 (s, 4H), 3.77-3.71 (m, 13H), 2.59 (t, J=6.8 Hz, 2H), 2.52 (t, J=6.6 Hz, 2H), 2.20-2.20 (m, 3H), 2.15-2.12 (m, 3H), 2.10 (s, 3H), 1.87-1.73 (m, 2H), 1.58-1.51 (m, 4H), 1.47 (s, 9H), 1.35-1.27 (m, 21H), 0.90-0.86 (m, 12H).

### Preparation 4

### 3-[2-[2-[2-[2-[(2R)-2,5,7,8-tetramethyl-2-[(4R,8R)-4,8,12-trimethyltridecyl]chroman-6-yl]oxyethoxy]ethoxy]ethoxy]ethoxy]propanoic acid

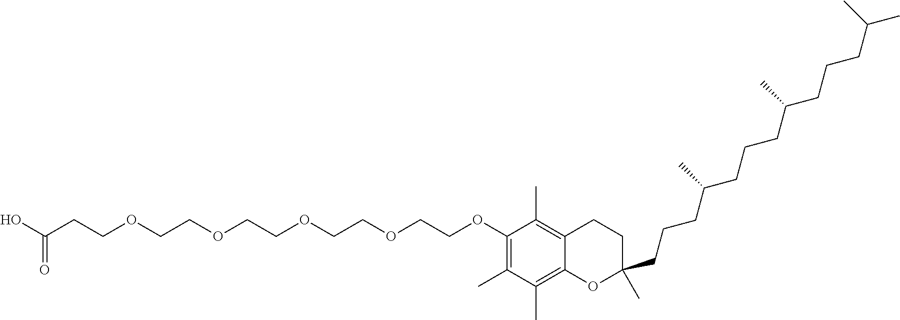 [SMILES: Cc1c(C)c2c(c(C)c1OCCOCCOCCOCCOCCC(=O)O)CC[C@@](C)(CCC[C@H](C)CCC[C@H](C)CCCC(C)C)O2]

Dissolved tert-butyl 3-[2-[2-[2-[2-[(2R)-2,5,7,8-tetramethyl-2-[(4R,8R)-4,8,12-trimethyltridecyl]chroman-6-yl]oxyethoxy]ethoxy]ethoxy]ethoxy]propanoate (3.33 g, 4.53 mmol) in 4M HCl in dioxane (22.6 mL, 90.6 mmol) and stirred for 16 hours at ambient temperature. Removed the solvent under reduced pressure to give the title compound as an off-white solid (3.08 g, 100%). ES/MS m/z 678.0 (M−H).

### Preparation 5

### (2S)-3-[Bis(4-methoxyphenyl)-phenyl-methoxy]-2-(9H-fluoren-9-ylmethoxycarbonylamino) propanoic acid

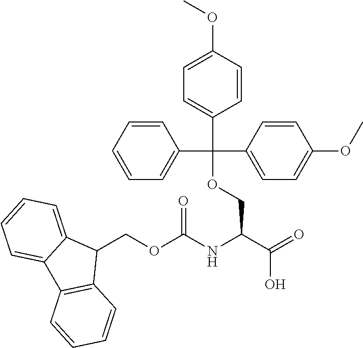 [SMILES: COc1ccc(C(OC[C@H](NC(=O)OCC2c3ccccc3-c3ccccc32)C(=O)O)(c2ccccc2)c2ccc(OC)cc2)cc1]

Added DIEA (64 mL, 0.366 mol) to a stirring solution of (2S)-2-(9H-fluoren-9-ylmethoxycarbonylamino)-3-hydroxy-propanoic acid (40 g, 0.122 mol) in dry DCM (400 mL) at 0° C. under an inert atmosphere. To this mixture, slowly added a solution of DMTCl (49.6 g, 0.146 mol) in DCM (200 mL). Allowed to warm to ambient temperature and stirred for 16 hours. After this time, diluted the reaction mixture with water and extracted with DCM. Dried organics over anhydrous sodium sulphate, filtered, and concentrated in vacuo. Washed the crude residue with 10% EtOAc/hexane and dried under vacuum to give the crude title compound as a pale brown solid (62 g, crude). TLC: 5% MeOH/CH₂Cl₂ (Rf 0.5) UV, 254 nM.

### Preparation 6

### 9H-Fluoren-9-ylmethyl N-[(1S)-1-[[bis(4-methoxyphenyl)-phenyl-methoxy]methyl]-2-[4-(hydroxymethyl)-1-piperidyl]-2-oxo-ethyl]carbamate

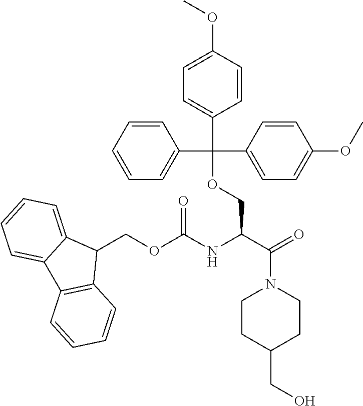 [SMILES: COc1ccc(C(OC[C@H](NC(=O)OCC2c3ccccc3-c3ccccc32)C(=O)N2CCC(CO)CC2)(c2ccccc2)c2ccc(OC)cc2)cc1]

Slowly added HBTU (78.3 g, 0.206 mol), HOBt (27.9 g, 0.206 mol), and piperidin-4-yl methanol (15.4 g, 0.134 mol) followed by TMP (15 mL, 0.113 mol) to a stirring solution of (2S)-3-[bis(4-methoxyphenyl)-phenyl-methoxy]-2-(9H-fluoren-9-ylmethoxycarbonylamino) propanoic acid (62 g, 0.103 mol) in DCM (750 mL) at 0° C. under inert atmosphere. Allowed the resulting reaction mixture to reach ambient temperature and stirred for 4 hours. After this time, diluted the mixture with water and extracted with DCM. Dried the organics over anhydrous sodium sulphate, filtered, and concentrated in vacuo. Purified the resulting residue via silica gel flash chromatography eluting with 20-40% EtOAc/hexane and 1% MeOH/DCM to give the title compound (40 g, 52% over two steps). ¹H NMR (DMSO-d₆) δ 7.88 (br d, J=7.5 Hz, 2H), 7.79-7.59 (m, 3H), 7.45-7.12 (m, 13H), 6.92-6.76 (m, 4H), 4.79-4.44 (m, 2H), 4.32 (br d, J=11.4 Hz, 2H), 4.20 (br s, 2H), 3.71 (s, 6H), 3.21 (br s, 4H), 2.99-2.79 (m, 1H), 2.69 (br s, 2H), 1.81-1.43 (m, 3H), 1.08-0.73 (m, 2H).

### Preparation 7

### (2S)-2-Amino-3-[bis(4-methoxyphenyl)-phenyl-methoxy]-1-[4-(hydroxymethyl)-1-piperidyl]propan-1-one

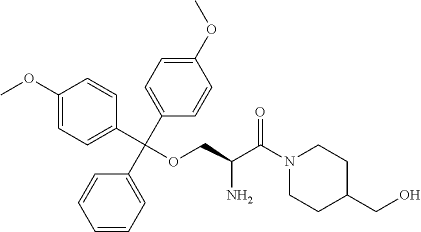 [SMILES: COc1ccc(C(OC[C@H](N)C(=O)N2CCC(CO)CC2)(c2ccccc2)c2ccc(OC)cc2)cc1]

Slowly added a solution of 20% piperidine in DMF (400 mL) to 9H-fluoren-9-ylmethyl N-[(1S)-1-[[bis(4-methoxyphenyl)-phenyl-methoxy]methyl]-2-[4-(hydroxymethyl)-1-piperidyl]-2-oxo-ethyl]carbamate (40 g, 0.055 mol) at 0° C. under inert atmosphere. Allowed the mixture to warm to ambient temperature and stirred for 1 hour. After this time, diluted the mixture with water and extracted with EtOAc. Dried organics over anhydrous sodium sulphate, filtered, and concentrated in vacuo. Purified the resulting residue via silica gel flash chromatography eluting with 1-8% MeOH/DCM to give the title compound as an off-white solid (13 g, 47%). ES/MS m z 1009.5 (2M+H).

### Preparation 8

### N-[2-[2-[2-[2-[3-[[(1S)-1-[[Bis(4-methoxyphenyl)-phenyl-methoxy]methyl]-2-[4-(hydroxymethyl)-1-piperidyl]-2-oxo-ethyl]amino]-3-oxo-propoxy]ethoxy]ethoxy]ethoxy]ethyl]hexadecanamide

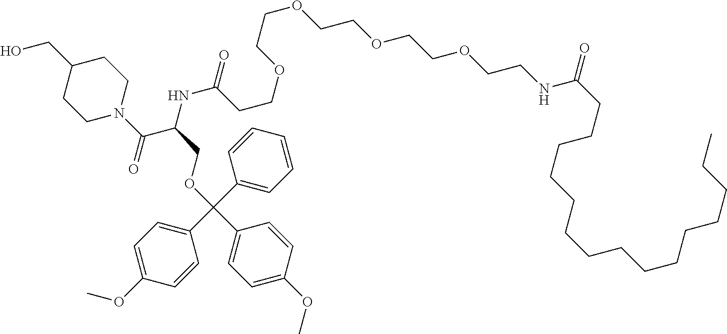 [SMILES: CCCCCCCCCCCCCCCC(=O)NCCOCCOCCOCCOCCC(=O)N[C@@H](COC(c1ccccc1)(c1ccc(OC)cc1)c1ccc(OC)cc1)C(=O)N1CCC(CO)CC1]

Combined 3-[2-[2-[2-[2-(hexadecanoylamino)ethoxy]ethoxy]ethoxy]ethoxy]propanoic acid (496 mg, 0.984 mmol), HOBt (146 mg, 1.08 mmol), HBTU (410 mg, 1.08 mmol), and DIEA (1.03 mL, 5.90 mmol) in DMF (9.84 mL) and stirred at ambient temperature for 10 minutes. Added (2S)-2-amino-3-[bis(4-methoxyphenyl)-phenyl-methoxy]-1-[4-(hydroxymethyl)-1-piperidyl]propan-1-one (546 mg, 1.08 mmol) to the mixture and stirred at ambient temperature for 16 hours. Partitioned the mixture between EtOAc and saturated aqueous sodium chloride solution. Separated the layers and washed the organics with saturated aqueous sodium chloride solution. Dried the organics over sodium sulfate, filtered, and concentrated in vacuo. Purified the resulting residue by silica gel flash chromatography eluting with 0-10% MeOH/DCM to give the title compound as an oil (327 mg, 34%). 1H NMR (DMSO-d₆) 8.21 (d, J=8.5 Hz, 1H), 7.80 (t, J=5.6 Hz, 1H), 7.37-7.28 (m, 4H), 7.23-7.20 (m, 5H), 6.88 (d, J=8.3 Hz, 4H), 5.06-5.02 (m, 1H), 4.51-4.49 (m, 1H), 4.45-4.40 (m, 1H), 3.97-3.93 (m, 1H), 3.74 (s, 5H), 3.63-3.56 (m, 2H), 3.49-3.48 (m, 4H), 3.47-3.45 (m, 7H), 3.40-3.35 (m, 2H), 3.30 (s, 1H), 3.23-3.13 (m, 7H), 2.41-2.33 (m, 2H), 2.04 (t, J=7.4 Hz, 2H), 1.74-1.69 (m, 3H), 1.51-1.44 (m, 2H), 1.26-1.24 (m, 24H), 1.00-0.97 (m, 1H), 0.88-0.82 (m, 5H).

### Preparation 9

### 4-[[1-[(2S)-3-[Bis(4-methoxyphenyl)-phenyl-methoxy]-2-[3-[2-[2-[2-[2-(hexadecanoylamino)ethoxy]ethoxy]ethoxy]ethoxy]propanoylamino]propanoyl]-4-piperidyl]methoxy]-4-oxo-butanoic acid

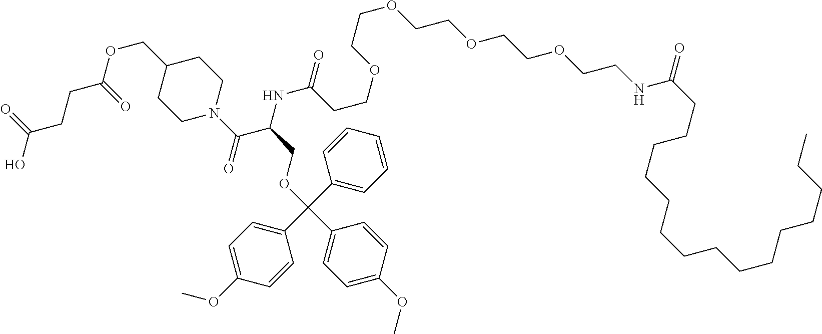 [SMILES: CCCCCCCCCCCCCCCC(=O)NCCOCCOCCOCCOCCC(=O)N[C@@H](COC(c1ccccc1)(c1ccc(OC)cc1)c1ccc(OC)cc1)C(=O)N1CCC(COC(=O)CCC(=O)O)CC1]

Combined N-[2-[2-[2-[2-[3-[[(1S)-1-[[bis(4-methoxyphenyl)-phenyl-methoxy]methyl]-2-[4-(hydroxymethyl)-1-piperidyl]-2-oxo-ethyl]amino]-3-oxo-propoxy]ethoxy]ethoxy]ethoxy]ethyl]hexadecanamide (320 mg, 0.323 mmol), DMAP (120 mg, 0.969 mmol), TEA (225 μL, 1.62 mmol), and succinic anhydride (64.7 mg, 0.646 mmol) in DCM (6.46 mL) and stirred the mixture for 16 hours at ambient temperature. Purified the mixture directly via silica gel flash chromatography, eluting with 0% to 40% MeOH/DCM, to give the title compound as a colorless oil (279 mg, 79%). ¹H NMR (DMSO-d₆) 12.65-12.64 (m, 1H), 8.24-8.19 (m, 1H), 7.80 (t, J=5.6 Hz, 1H), 7.37-7.28 (m, 4H), 7.24-7.20 (m, 5H), 6.88 (d, J=8.6 Hz, 4H), 5.05-5.01 (m, 1H), 4.44-4.40 (m, 1H), 3.97-3.95 (m, 3H), 3.74 (s, 6H), 3.61-3.56 (m, 2H), 3.49-3.45 (m, 11H), 3.38 (t, J=5.9 Hz, 3H), 3.22-3.14 (m, 6H), 2.48-2.31 (m, 7H), 2.04 (t, J=7.4 Hz, 2H), 1.90-1.87 (m, 5H), 1.24 (s, 23H), 0.98-0.96 (m, 1H), 0.87-0.82 (m, 4H).

### Preparation 10

### 4-[[1-[(2S)-3-[Bis(4-methoxyphenyl)-phenyl-methoxy]-2-[3-[2-[2-[2-[2-[(2R)-2,5,7,8-tetramethyl-2-[(4R,8R)-4,8,12-trimethyltridecyl]chroman-6-yl]oxyethoxy]ethoxy]ethoxy]ethoxy]propanoylamino]propanoyl]-4-piperidyl]methoxy]-4-oxo-butanoic acid

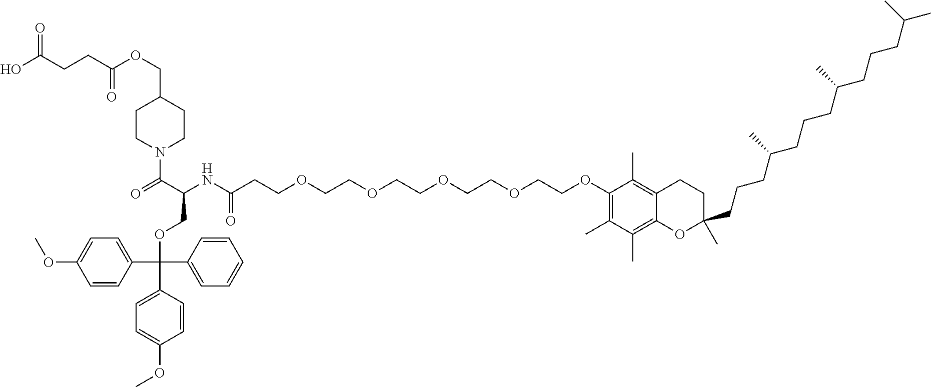 [SMILES: COc1ccc(C(OC[C@H](NC(=O)CCOCCOCCOCCOCCOc2c(C)c(C)c3c(c2C)CC[C@@](C)(CCC[C@H](C)CCC[C@H](C)CCCC(C)C)O3)C(=O)N2CCC(COC(=O)CCC(=O)O)CC2)(c2ccccc2)c2ccc(OC)cc2)cc1]

Combined 3-[2-[2-[2-[2-[(2R)-2,5,7,8-tetramethyl-2-[(4R,8R)-4,8,12-trimethyltridecyl]chroman-6-yl]oxyethoxy]ethoxy]ethoxy]ethoxy]propanoic acid (1.20 g, 1.80 mmol), HOBt (260 mg, 1.90 mmol), HBTU (740 mg, 1.90 mmol), and DIEA (1.80 mL, 11.0 mmol) in DMF (18.0 mL) and stirred at ambient temperature for 10 minutes. Added (2S)-2-amino-3-[bis(4-methoxyphenyl)-phenyl-methoxy]-1-[4-(hydroxymethyl)-1-piperidyl]propan-1-one (980 mg, 1.90 mmol) to the mixture and stirred at ambient temperature for 16 hours. Partitioned the mixture between EtOAc and saturated aqueous sodium chloride solution. Separated the layers and washed the organics with saturated aqueous sodium chloride solution. Dried the organic layer over sodium sulfate, filtered, and concentrated in vacuo. Purified the resulting residue by silica gel flash chromatography, eluting with 0-10% MeOH/DCM, to give N-[(1S)-1-[[bis(4-methoxyphenyl)-phenyl-methoxy]methyl]-2-[4-(hydroxymethyl)-1-piperidyl]-2-oxo-ethyl]-3-[2-[2-[2-[2-[(2R)-2,5,7,8-tetramethyl-2-[(4R,8R)-4,8,12-trimethyltridecyl]chroman-6-yl]oxyethoxy]ethoxy]ethoxy]ethoxy]propenamide as a yellow oil.

Combined N-[(1S)-1-[[bis(4-methoxyphenyl)-phenyl-methoxy]methyl]-2-[4-(hydroxymethyl)-1-piperidyl]-2-oxo-ethyl]-3-[2-[2-[2-[2-[(2R)-2,5,7,8-tetramethyl-2-[(4R,8R)-4,8,12-trimethyltridecyl]chroman-6-yl]oxyethoxy]ethoxy]ethoxy]ethoxy]propenamide (1.45 g, 1.24 mmol), DMAP (456 mg, 3.73 mmol), TEA (867 μL, 6.22 mmol), and succinic anhydride (249 mg, 2.49 mmol) in DCM (24.9 mL) and stirred for 16 hours at ambient temperature. Concentrated in vacuo and purified the resulting residue via silica gel flash chromatography, eluting with 0-40% MeOH/DCM, to give the title compound as an oil (1.36 g, 60%). ES/MS m/z 1264.4 (M−H).

### Preparation 11

### [[4-[[1-[(2S)-3-[Bis(4-methoxyphenyl)-phenyl-methoxy]-2-[3-[2-[2-[2-[2-(hexadecanoylamino)ethoxy]ethoxy]ethoxy]ethoxy]propanoylamino]propanoyl]-4-piperidyl]methoxy]-4-oxo-butanoyl]amino] on CPG

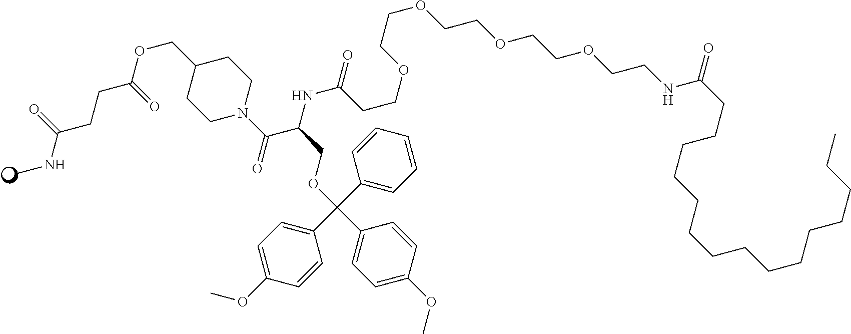 [SMILES: CCCCCCCCCCCCCCCC(=O)NCCOCCOCCOCCOCCC(=O)N[C@@H](COC(c1ccccc1)(c1ccc(OC)cc1)c1ccc(OC)cc1)C(=O)N1CCC(COC(=O)CCC(=O)NC)CC1]

Dissolved 4-[[1-[(2S)-3-[bis(4-methoxyphenyl)-phenyl-methoxy]-2-[3-[2-[2-[2-[2-(hexadecanoylamino)ethoxy]ethoxy]ethoxy]ethoxy]propanoylamino]propanoyl]-4-piperidyl]methoxy]-4-oxo-butanoic acid (270 mg, 0.248 mmol) in ACN (12.5 mL) and transferred the solution to a fritted glass dropping funnel. Added DIEA (150 μL, 0.860 mmol) and HBTU (190 mg, 0.500 mmol) to the solution and shook the mixture at ambient temperature for 10 minutes. Added native amino LCAA CPG 500 Å (1.92 g, 129 μmol/g) to the solution and shook the mixture at 500 RPM for 16 hours at ambient temperature. Drained the CPG and dried under nitrogen for 5 minutes. Washed the CPG with DCM (50 mL), 10% MeOH/DCM (50 mL), and then diethyl ether (50 mL). Dried the CPG for 30 minutes under nitrogen and then resuspended in pyridine (15 mL). Added acetic anhydride (3.30 mL, 35.0 mmol) and TEA (0.50 mL) and shook the mixture at 500 RPM for 2 hours at ambient temperature. Drained the CPG and dried for 5 minutes under nitrogen. Washed the CPG with DCM (50 mL), 10% MeOH/DCM (50 mL), and then diethyl ether (50 mL). Dried the CPG for 45 minutes under nitrogen and determined the ligand loading at 505 nm to give the title compound (1.92 g, 75.5 μmol/g).

### Preparation 12

### [[4-[[1-[(2S)-3-[Bis(4-methoxyphenyl)-phenyl-methoxy]-2-[3-[2-[2-[2-[2-[(2R)-2,5,7,8-tetramethyl-2-[(4R,8R)-4,8,12-trimethyltridecyl]chroman-6-yl]oxyethoxy]ethoxy]ethoxy]ethoxy]propanoylamino]propanoyl]-4-piperidyl]methoxy]-4-oxo-butanoyl]amino] on CPG

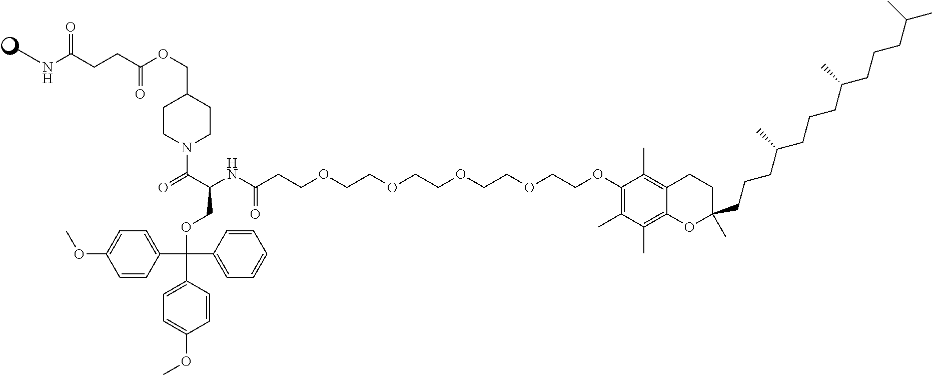 [SMILES: CNC(=O)CCC(=O)OCC1CCN(C(=O)[C@H](COC(c2ccccc2)(c2ccc(OC)cc2)c2ccc(OC)cc2)NC(=O)CCOCCOCCOCCOCCOc2c(C)c(C)c3c(c2C)CC[C@@](C)(CCC[C@H](C)CCC[C@H](C)CCCC(C)C)O3)CC1]

Prepared the title compound from 4-[[1-[(2S)-3-[bis(4-methoxyphenyl)-phenyl-methoxy]-2-[3-[2-[2-[2-[2-[(2R)-2,5,7,8-tetramethyl-2-[(4R,8R)-4,8,12-trimethyltridecyl]chroman-6-yl]oxyethoxy]ethoxy]ethoxy]ethoxy]propanoylamino]propanoyl]-4-piperidyl]methoxy]-4-oxo-butanoic acid in a manner essentially analogous to Preparation 11. Determined the ligand loading at 505 nm to give the title compound (4.01 g, 66.9 μmol/g).

### Preparation 13

### 3-[[(2R,3R,4R,5R)-2-[[Bis(4-methoxyphenyl)-phenyl-methoxy]methyl]-4-hexadecoxy-5-(2-hydroxy-4-oxo-pyrimidin-1-yl)THF-3-yl]oxy-(diisopropylamino)phosphanyl]oxypropanenitrile

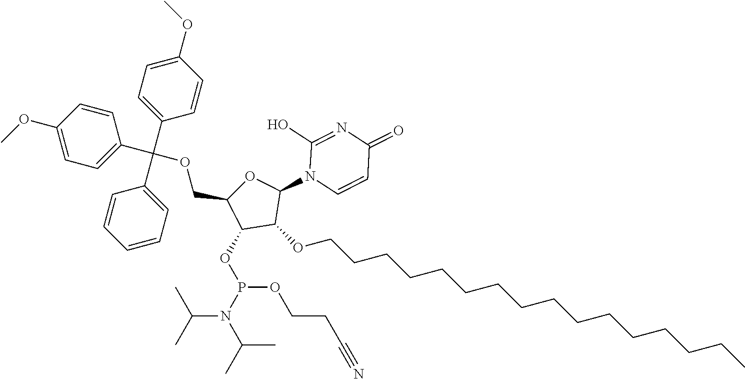 [SMILES: CCCCCCCCCCCCCCCCO[C@@H]1[C@H](OP(OCCC#N)N(C(C)C)C(C)C)[C@@H](COC(c2ccccc2)(c2ccc(OC)cc2)c2ccc(OC)cc2)O[C@H]1n1ccc(=O)nc1O]

Prepared the title compound according to the protocols described in WO2019217459. ¹H NMR (CD₃CN): 7.86-7.73 (m, 1H), 7.51-7.43 (m, 2H), 7.40-7.23 (m, 7H), 6.95-6.87 (m, 4H), 5.90-5.84 (m, 1H), 5.29-5.21 (m, 1H), 4.54-4.40 (m, 1H), 4.21-4.13 (m, 1H), 4.10-3.56 (m, 13H), 3.50-3.34 (m, 2H), 2.75-2.62 (m, 1H), 2.55 (t, J=6.0 Hz, 1H), 1.66-1.51 (m, 2H), 1.40-1.14 (m, 35H), 1.08 (d, J=6.8 Hz, 3H), 0.91 (t, J=6.8 Hz, 3H). ³¹P NMR (CD₃CN): 149.6, 149.2.

### Preparation 14

### N-[9-[(2R,3R,4R,5R)-5-[[Bis(4-methoxyphenyl)-phenyl-methoxy]methyl]-4-[2-cyanoethoxy-(diisopropylamino)phosphanyl]oxy-3-hexadecoxy-tetrahydrofuran-2-yl]purin-6-yl]benzamide

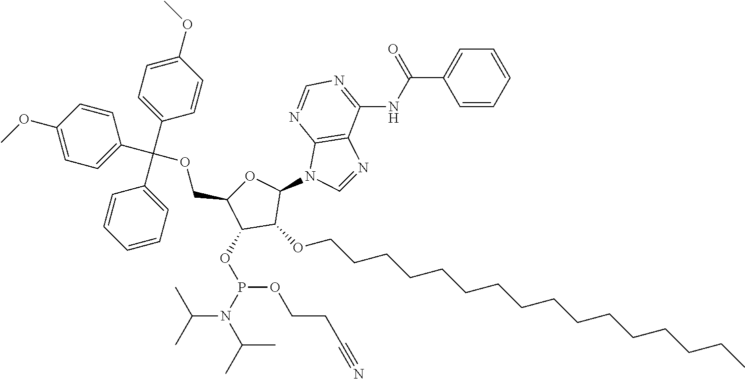 [SMILES: CCCCCCCCCCCCCCCCO[C@@H]1[C@H](OP(OCCC#N)N(C(C)C)C(C)C)[C@@H](COC(c2ccccc2)(c2ccc(OC)cc2)c2ccc(OC)cc2)O[C@H]1n1cnc2c(NC(=O)c3ccccc3)ncnc21]

Prepared the title compound according to the protocols described in WO2019217459. ¹H-NMR (CD₃CN) δ 9.37 (s, 1H), 8.57 (d, J=9.4 Hz, 1H), 8.27 (d, J=10.3 Hz, 1H), 7.99 (d, J=7.6 Hz, 2H), 7.61 (d, J=7.4 Hz, 1H), 7.52 (t, J=7.6 Hz, 2H), 7.42 (t, J=7.3 Hz, 2H), 7.34-7.16 (m, 7H), 6.85-6.77 (m, 4H), 6.1 1 (dd, J=5.0, 2.5 Hz, 1H), 4.80 (m, 1H), 4.69 (m, 1H), 4.32 (m, 1H), 3.97-3.78 (m, 1H), 3.74 (d, J=3.1 Hz, 7H), 3.64 (m, 4H), 3.56-3.40 (m, 2H), 3.33 (m, 1H), 2.73-2.59 (m, 1H), 2.50 (t, J=6.0 Hz, 1H), 1.52-1.45 (m, 2H), 1.33-1.12 (m, 37H), 1.09 (d, J=6.8 Hz, 3H), 0.87 (t, J=6.8 Hz, 3H). ³¹P NMR (CD₃CN) δ 151.19, 150.78.

### Preparation 15

### N-[1-[(2R,3R,4R,5R)-5-[[Bis(4-methoxyphenyl)-phenyl-methoxy]methyl]-4-[2-cyanoethoxy-(diisopropylamino)phosphanyl]oxy-3-hexadecoxy-tetrahydrofuran-2-yl]-2-oxo-pyrimidin-4-yl]acetamide

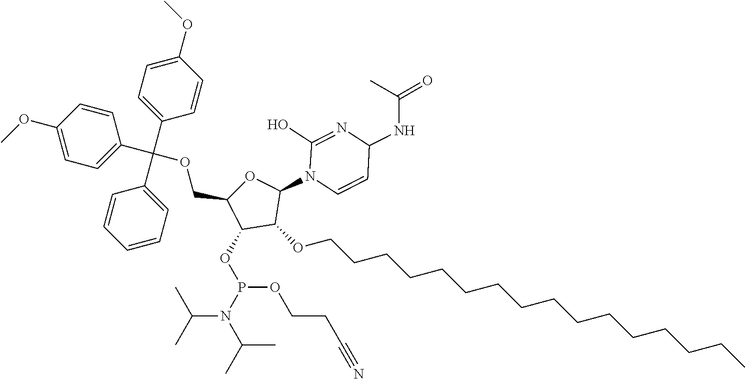 [SMILES: CCCCCCCCCCCCCCCCO[C@@H]1[C@H](OP(OCCC#N)N(C(C)C)C(C)C)[C@@H](COC(c2ccccc2)(c2ccc(OC)cc2)c2ccc(OC)cc2)O[C@H]1N1C=CC(NC(C)=O)N=C1O]

Prepared the title compound according to the protocols described in WO2019217459. ¹H-NMR (CD₃CN) 9.15 (s, 1H), 8.46 (dd, J=7.5 Hz, 1H), 7.95 (d, J=7.6 Hz, 2H), 7.63 (t, J=7.5 Hz, 1H), 7.57-7.41 (m, 5H), 7.41-7.31 (m, 6H), 7.28 (m, 1H), 7.04 (d, J=15.8 Hz, 1H), 6.90 (t, J=7.9 Hz, 4H), 5.90 (d, J=7.8 Hz, 1H), 4.51 (m, 1H), 4.20 (dd, J=10.6, 8.1 Hz, 1H), 4.04 (dd, J=31.3, 4.6 Hz, 1H), 3.91-3.81 (m, 2H), 3.79 (d, J=3.1 Hz, 6H), 3.74 (m, 2H), 3.69-3.41 (m, 6H), 2.67-2.59 (m, 1H), 2.54-2.48 (m, 1H), 1.58 (m, 2H), 1.36 (m, 2H), 1.25 (d, J=4.7 Hz, 26H), 1.21-1.09 (m, 10H), 1.04 (d, J=6.8 Hz, 3H), 0.87 (t, J=6.8 Hz, 3H). ³¹P NMR (CD₃CN) δ 151.10, 150.19.

### Preparation 16

### N-[9-[(2R,3R,4R,5R)-5-[[Bis(4-methoxyphenyl)-phenyl-methoxy]methyl]-4-[2-cyanoethoxy-(diisopropylamino)phosphanyl]oxy-3-hexadecoxy-tetrahydrofuran-2-yl]-6-oxo-1H-purin-2-yl]-2-methyl-propanamide

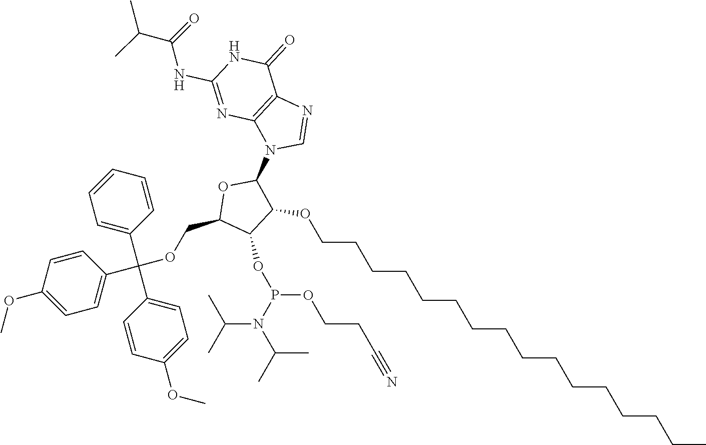 [SMILES: CCCCCCCCCCCCCCCCO[C@@H]1[C@H](OP(OCCC#N)N(C(C)C)C(C)C)[C@@H](COC(c2ccccc2)(c2ccc(OC)cc2)c2ccc(OC)cc2)O[C@H]1n1cnc2c(=O)[nH]c(NC(=O)C(C)C)nc21]

Prepared the title compound according to the protocols described in WO2019217459. ¹H-NMR (CDCl₃) δ 12.01-11.96 (m, 1H), 7.82-7.78 (m, 1H), 7.59-7.53 (m, 1H), 7.47-7.42 (m, 1H), 7.41-7.37 (m, 2H), 7.34-7.29 (m, 2H), 7.27-7.22 (m, 3H), 6.85-6.80 (m, 4H), 5.99-5.82 (m, 1H), 4.40-4.36 (m, 1H), 4.17-4.11 (m, 1H), 3.80-3.77 (m, 6H), 3.76-3.68 (m, 6H), 3.22-3.17 (m, 1H), 2.84-2.79 (m, 1H), 1.60-1.54 (m, 4H), 1.35-1.30 (m, 6H), 1.27 (s, 19H), 1.24-1.15 (m, 13H), 1.06-1.03 (m, 5H), 0.93-0.88 (m, 6H), 0.74-0.70 (m, 1H). ³¹P NMR (CDCl₃) δ 150.20, 149.92.

### Preparation 17

### 4-[[1-[(2S)-2-[6-[[(2S)-2-[5-[3-Acetamido-4,5-diacetoxy-6-(acetoxymethyl)tetrahydropyran-2-yl]oxypentanoylamino]-5-[bis[2-[5-[3-acetamido-4,5-diacetoxy-6-(acetoxymethyl)tetrahydropyran-2-yl]oxypentanoylamino]ethyl]amino]-5-oxo-pentanoyl]amino]hexanoylamino]-3-[bis(4-methoxyphenyl)-phenyl-methoxy]propanoyl]-4-piperidyl]methoxy]-4-oxo-butanoic acid

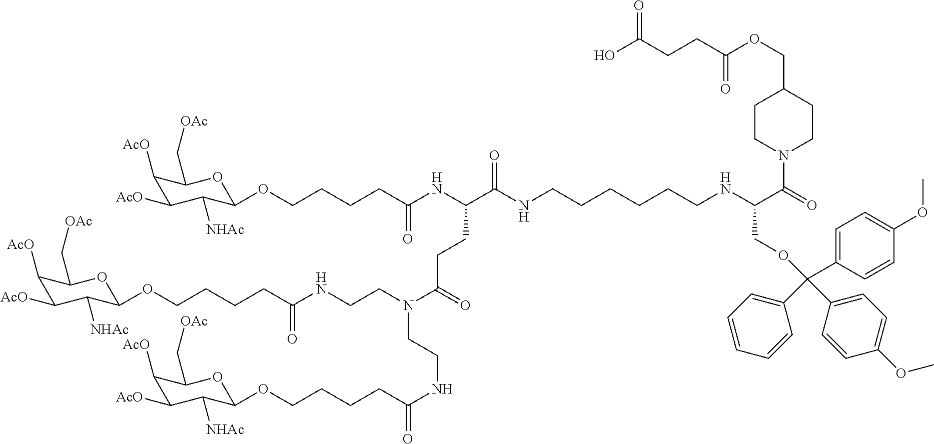 [SMILES: COc1ccc(C(OC[C@H](NCCCCCCNC(=O)[C@H](CCC(=O)N(CCNC(=O)CCCCOC2OC(COC(C)=O)C(OC(C)=O)C(OC(C)=O)C2NC(C)=O)CCNC(=O)CCCCOC2OC(COC(C)=O)C(OC(C)=O)C(OC(C)=O)C2NC(C)=O)NC(=O)CCCCOC2OC(COC(C)=O)C(OC(C)=O)C(OC(C)=O)C2NC(C)=O)C(=O)N2CCC(COC(=O)CCC(=O)O)CC2)(c2ccccc2)c2ccc(OC)cc2)cc1]

Prepared the title compound according to the protocols described in WO2022271806. ES/MS m/z 1109.60 (M−2H)/2.

### Preparation 18

### Resin Loading

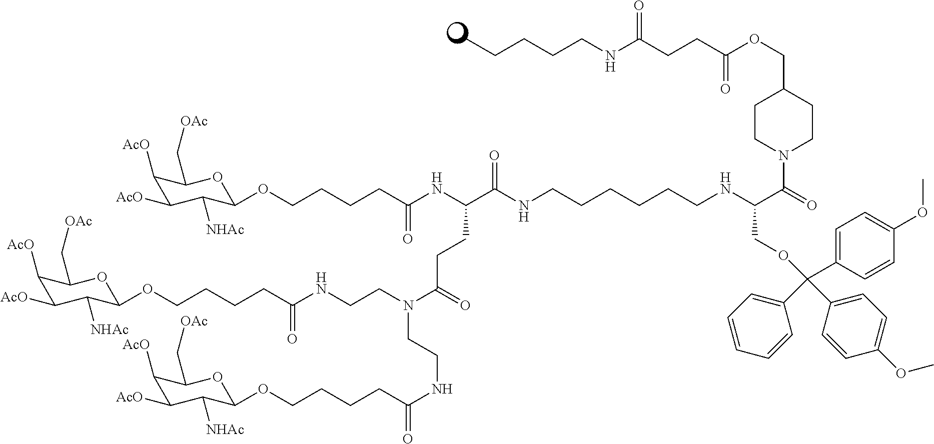 [SMILES: CCCCCNC(=O)CCC(=O)OCC1CCN(C(=O)[C@H](COC(c2ccccc2)(c2ccc(OC)cc2)c2ccc(OC)cc2)NCCCCCCNC(=O)[C@H](CCC(=O)N(CCNC(=O)CCCCOC2OC(COC(C)=O)C(OC(C)=O)C(OC(C)=O)C2NC(C)=O)CCNC(=O)CCCCOC2OC(COC(C)=O)C(OC(C)=O)C(OC(C)=O)C2NC(C)=O)NC(=O)CCCCOC2OC(COC(C)=O)C(OC(C)=O)C(OC(C)=O)C2NC(C)=O)CC1]

Prepared the title compound from 4-[[1-[(2S)-2-[6-[[(2S)-2-[5-[3-acetamido-4,5-diacetoxy-6-(acetoxymethyl)tetrahydropyran-2-yl]oxypentanoylamino]-5-[bis[2-[5-[3-acetamido-4,5-diacetoxy-6-(acetoxymethyl)tetrahydropyran-2-yl]oxypentanoylamino]ethyl]amino]-5-oxo-pentanoyl]amino]hexanoylamino]-3-[bis(4-methoxyphenyl)-phenyl-methoxy]propanoyl]-4-piperidyl]methoxy]-4-oxo-butanoic acid according to the protocols described in WO2022271806. The resin loading was calculated to be 34.7 μmol/g.

### Example 2. Synthesis of SARM1 RNAi Agents and Conjugated RNAi Agents

Single strands (sense and antisense) of the RNA duplexes were synthesized on solid support via a MerMade™ 12 (LGC Biosearch Technologies). The sequences of the sense and antisense strands were shown in Table 2. The oligonucleotides were synthesized via phosphoramidite chemistry at either 5, 10, 25 or 50 μmol scales.

For the sense strands, the types of solid supports were universal CPG: (3′-Piperidinol-PEG-Palmitate) and (3′-Piperidinol-PEG-Tocopherol) were synthesized in house (see Example 1) while the Universal UnyLinker (Chemgenes, Catalog No. AT273-27), 3′-Teg-Tocopherol (LGC Biosearch Technologies, Catalog No. BG7-1190), and 3′-Cholesterol-TEG CPG (Glen Research, Catalog No. 20-2975) were purchased commercially. For all the antisense strands, commercially available standard support mA was utilized. Standard reagents were used in the oligo synthesis (Table 7), where 0.1M xanthane hydride in pyridine was used as the sulfurization reagent and 20% DEA in ACN was used as an auxiliary wash post synthesis. All monomers (Table 8) were made at 0.1M in ACN and contained a molecular sieves trap bag.

The oligonucleotides were cleaved and deprotected (C/D) at 45° C. for 20 hours. The sense strands were C/D from the CPG using ammonia hydroxide (28-30%, cold), whereas 3% DEA in ammonia hydroxide (28-30%, cold) was used for the antisense strands. C/D was determined complete by IP-RP LCMS when the resulting mass data confirmed the identity of sequence. Dependent on scale, the CPG was filtered via 0.45 um PVDF syringeless filter, 0.22 um PVDF Steriflip® vacuum filtration or 0.22 um PVDF Stericup® Quick release. The CPG was back washed/rinsed with either 30% ACN/RNAse free water or 30% EtOH/RNAse free water then filtered through the same filtering device and combined with the first filtrate. This was repeated twice. The material was then divided evenly into 50 mL falcon tubes to remove organics via Genevac™. After concentration, the crude oligonucleotides were diluted back to synthesized scale with RNAse free water and filtered either by 0.45 μm PVDF syringeless filter, 0.22 μm PVDF Steriflip® vacuum filtration or 0.22 μm PVDF Stericup® Quick release.

The crude oligonucleotides were purified via AKTA™ Pure purification system using either anion-exchange (AEX) or reverse phase (RP) a source 15Q-RP column. For AEX, an ES Industry Source™ 15Q column maintaining column temperature at 65° C. with MPA: 20 mM NaH₂PO₄, 15% ACN, pH 7.4 and MPB: 20 mM NaH₂PO₄, 1M NaBr, 15% ACN, pH 7.4. For RP, a Source™ 15Q-RP column with MPA: 50 mM NaOAc with 10% ACN and MPB: 50 mM NaOAc with 80% ACN. In all cases, fractions which contained a mass purity greater than 85% without impurities >5% where combined.

The purified oligonucleotides were desalted using 15 mL 3K MWCO centrifugal spin tubes at 3500×g for ˜30 min. The oligonucleotides were rinsed with RNAse free water until the eluent conductivity reached <100 usemi/cm. After desalting was complete, 2-3 mL of RNAse free water was added then aspirated 10×, the retainment was transferred to a 50 mL falcon tube, this was repeated until complete transfer of oligo by measuring concentration of compound on filter via nanodrop. The final oligonucleotide was then nano filtered 2× via 15 mL 100K MWCO centrifugal spin tubes at 3500×g for 2 min. The final desalted oligonucleotides were analyzed for concentration (nano drop at A260), characterized by IP-RP LCMS for mass purity and UPLC for UV-purity.

For the preparation of duplexes, equimolar amounts of sense and antisense strand were combined and heated at 65° C. for 10 minutes then slowly cooled to ambient temperature over 40 minutes. Integrity of the duplex was confirmed by UPLC analysis and characterized by LCMS using IP-RP. All duplexes were nano filtered then endotoxin levels measured via Charles River Endosafe® Cartridge Device to give the final compounds of conjugated RNAi (Table 9). For in vivo analysis, the appropriate amount of duplex was lyophilized then reconstituted in 1×PBS for rodent studies and a CSF for non-human primate studies.

TABLE 7
Oligonucleotide Synthesis Reagents
Reagents

Activator Solution (0.5M ETT in ACN)
Cap A (Acetic Anhydride, Pyridine in THF, 1:1:8)
Cap B (1-Methylimidazole in THF, 16:84)
Oxidation Solution (0.02M Iodine in THF/Pyridine/Water, 70:20:10)
Deblock Solution, 3% TCA in DCM (w/v)
Acetonitrile (Anhydrosolv, Water max. 10 ppm)
Xanthane Hydride (0.1M in Pyridine)
Diethylamine (20% in Acetonitrile)

TABLE 8
Phosphoramidites

| Phosphoramidite | Abbreviation | Supplier | Catalog # | CAS |
|---|---|---|---|---|
| DMT-2′-F-A(Bz)- | fA | Hongene | PD1-001 | 136834-22-5 |
| CE Phosphoamidite | | | | |
| DMT-2′-F-C(Ac)- | fC | Hongene | PD3-001 | 159414-99-0 |
| CE Phosphoamidite | | | | |
| DMT-2′-F-G(iBu)- | fG | Hongene | PD2-002 | 144089-97-4 |
| CE Phosphoamidite | | | | |
| DMT-2′-F-U-CE | fU | Hongene | PD5-001 | 146954-75-8 |
| Phosphoamidite | | | | |
| DMT-2′-O-Me- | mA | Hongene | PR1-001 | 110782-31-5 |
| A(Bz)-CE | | | | |
| Phosphoamidite | | | | |
| DMT-2′-O-Me- | mC | Hongene | PR3-001 | 199593-09-4 |
| C(Ac)-CE | | | | |
| Phosphoamidite | | | | |
| DMT-2′-O-Me- | mG | Hongene | PR2-002 | 150780-67-9 |
| G(iBu)-CE | | | | |
| Phosphoamidite | | | | |
| DMT-2′-O-Me-U- | mU | Hongene | PR5-001 | 110764-79-9 |
| CE Phosphoamidite | | | | |
| 5′bis(POM) vinyl | POM-VPmU | Hongene | PR5-032 | BVPMUP23B2A1 |
| phosphate-2′-Ome- | | | | |
| U3′CE | | | | |
| phosphoroamidite | | | | |
| Reverse Abasic | iAb | Chemgenes | ANP-1422 | 401813-16-9 |
| phosphoroamidite | | | | |
| Abasic | Ab | Chemgenes | ANP-7058 | 129821-76-7 |
| phosphoroamidite | | | | |
| Uhd | Uhd | Lilly | | |
| Chd | Chd | Lilly | | |
| Ahd | Ahd | Lilly | | |
| Ghd | Ghd | Lilly | | |

TABLE 9
Conjugated SARM1 RNAi Agents

| Conjugated | RNAi | | | MW | MW |
|---|---|---|---|---|---|
| RNAi | Agent | | | Cal. | Obs. |
| Agent No. | No. | Strand | LDP | (g/mol) | (g/mol) |
| 1 | 13 | S: SEQ ID NO: 13 | 1* | 7618.5 | 7619.1 |
| | | AS: SEQ ID NO: 14 | | 7880.2 | 7881.3 |
| 2 | 14 | S: SEQ ID NO: 15 | 1 | 7603.5 | 7604.7 |
| | | AS: SEQ ID NO: 16 | | 7865.2 | 7866.2 |
| 3 | 15 | S: SEQ ID NO: 17 | 1 | 7546.5 | 7547.4 |
| | | AS: SEQ ID NO: 18 | | 7843.2 | 7844.4 |
| 4 | 16 | S: SEQ ID NO: 19 | 1 | 7641.6 | 7642.7 |
| | | AS: SEQ ID NO: 20 | | 7825.2 | 7827.1 |
| 5 | 17 | S: SEQ ID NO: 21 | 1 | 7761.6 | 7762.3 |
| | | AS: SEQ ID NO: 22 | | 7698.1 | 7699.4 |
| 6 | 18 | S: SEQ ID NO: 23 | 1 | 7602.5 | 7603.2 |
| | | AS: SEQ ID NO: 24 | | 7825.2 | 7826.5 |
| 7 | 7 | S: SEQ ID NO: 13 | 8* | 7675.6 | 7675.8 |
| | | AS: SEQ ID NO: 33 | | 7884.2 | 7884.8 |
| 8 | 8 | S: SEQ ID NO: 15 | 8 | 7660.5 | 7660.9 |
| | | AS: SEQ ID NO: 27 | | 7869.2 | 7869.5 |
| 9 | 9 | S: SEQ ID NO: 17 | 8 | 7603.5 | 7604.0 |
| | | AS: SEQ ID NO: 45 | | 7847.1 | 7847.3 |
| 10 | 10 | S: SEQ ID NO: 19 | 8 | 7698.6 | 7698.7 |
| | | AS: SEQ ID NO: 46 | | 7829.2 | 7829.2 |
| 11 | 11 | S: SEQ ID NO: 21 | 8 | 7818.7 | 7818.5 |
| | | AS: SEQ ID NO: 39 | | 7702.1 | 7702.3 |
| 12 | 12 | S: SEQ ID NO: 23 | 8 | 7659.6 | 7660.0 |
| | | AS: SEQ ID NO: 47 | | 7829.2 | 7829.7 |
| 13 | 8 | S: SEQ ID NO: 15 | 9* | 8406.2 | 8407.6 |
| | | AS: SEQ ID NO: 27 | | 7869.2 | 7870.5 |
| 14 | 19 | S: SEQ ID NO: 28 | 9 | 8418.2 | 8418.6 |
| | | AS: SEQ ID NO: 29 | | 7857.2 | 7857.2 |
| 15 | 20 | S: SEQ ID NO: 28 | 9 | 8418.2 | 8418.7 |
| | | AS: SEQ ID NO: 30 | | 7857.2 | 7857.3 |
| 16 | 21 | S: SEQ ID NO: 28 | 9 | 8418.2 | 8418.7 |
| | | AS: SEQ ID NO: 31 | | 7857.2 | 7857.2 |
| 17 | 22 | S: SEQ ID NO: 28 | 9 | 8418.2 | 8418.6 |
| | | AS: SEQ ID NO: 32 | | 7893.3 | 7893.4 |
| 18 | 7 | S: SEQ ID NO: 13 | 9 | 8421.2 | 8421.9 |
| | | AS: SEQ ID NO: 33 | | 7884.2 | 7885.7 |
| 19 | 23 | S: SEQ ID NO: 34 | 9 | 8433.2 | 8433.7 |
| | | AS: SEQ ID NO: 35 | | 7872.2 | 7872.5 |
| 20 | 24 | S: SEQ ID NO: 34 | 9 | 8433.2 | 8433.5 |
| | | AS: SEQ ID NO: 36 | | 7872.2 | 7872.6 |
| 21 | 25 | S: SEQ ID NO: 34 | 9 | 8433.2 | 8433.5 |
| | | AS: SEQ ID NO: 37 | | 7872.2 | 7872.5 |
| 22 | 26 | S: SEQ ID NO: 34 | 9 | 8433.2 | 8433.5 |
| | | AS: SEQ ID NO: 38 | | 7908.3 | 7908.6 |
| 23 | 11 | S: SEQ ID NO: 21 | 9 | 8564.3 | 8564.3 |
| | | AS: SEQ ID NO: 39 | | 7702.1 | 7702.3 |
| 24 | 27 | S: SEQ ID NO: 40 | 9 | 8576.3 | 8576.9 |
| | | AS: SEQ ID NO: 41 | | 7690.0 | 7690.3 |
| 25 | 28 | S: SEQ ID NO: 40 | 9 | 8576.3 | 8576.8 |
| | | AS: SEQ ID NO: 42 | | 7690.0 | 7690.4 |
| 26 | 29 | S: SEQ ID NO: 40 | 9 | 8576.3 | 8576.8 |
| | | AS: SEQ ID NO: 43 | | 7690.0 | 7690.3 |
| 27 | 30 | S: SEQ ID NO: 40 | 9 | 8576.3 | 8577.1 |
| | | AS: SEQ ID NO: 44 | | 7726.1 | 7726.3 |

“S” means the sense strand; “AS” means the antisense strand.
*LDP 1, 8, 9 is linked to the 3′ end of the sense strand. AS SEQ ID NOs: 14, 16, 18, 20, 22, and 24 include a 5′-vinylphosphonate; and all the other AS in this table include a 5′-phosphate.

### Example 3. Characterization of SARM1 RNAi Agents

Selected SARM1 RNAi agents were tested in vitro for SARM1 inhibition in cultured cells, including SH-SY5Y cells and/or human induced pluripotent stem cells (hiPSC).

### Materials and Methods

SH-SY5Y Cell Culture and RNAi Treatment and Analysis: SH-SY5Y cells (ATCC CRL-2266) were derived from the SK-N-SH neuroblastoma cell line (Ross, R. A., et al., 1983. J Natl Cancer Inst 71, 741-747). The base medium was composed of a 1:1 mixture of ATCC-formulated Eagle's Minimum Essential Medium, (Cat No. 30-2003), and F12 Medium. The complete growth medium was supplemented with 1000 fetal bovine serum, 1× amino acids, 1× sodium bicarbonate, and 1× penicillin-streptomycin (Gibco) and cells incubated at 37° C. in a humidified atmosphere of 5% CO₂. On Day One, SH-SY5Y cells were plated in 96 well fibronectin coated tissue culture plates and allowed to attach overnight. On Day Two, complete media was removed and replaced with RNAi agent in serum free media. Cells were incubated with RNAi agent for 72 hours before analysis of gene expression. Analysis of changes in gene expression in RNAi treated SH-SY5Y cells was measured using Cells-to-C_{T} Kits following the manufacturer's protocol (ThermoFisher A35377). Predesigned gene expression assays (supplied as 20× mixtures) were selected from Applied Bio-systems (Foster City, CA, USA). The efficiencies of these assays (ThermoFisher Hs00240906_m1 SARM1 and ThermoFisher Hs99999905_m1 GAPDH) were characterized with a dilution series of cDNA. RT-QPCR was performed in MicroAmp Optical 384-well reaction plates using QuantStudio 7 Flex system. The delta-delta CT method of normalizing to the housekeeping gene GAPDH was used to determine relative amounts of gene expression. GraphPad Prism v9.0 was used to determine IC50 with a four parameter logistic fit.

Human Induced Pluripotent Stem Cell-derived Neuron (hiPSC Neuron) Culture and RNAi Treatment and Analysis: Doxycycline-inducible Neurogenin2 (NGN2) human induced Pluripotent Stem Cells (hiPSC) were developed by Bioneer for Eli Lilly. The hiPSC were doxycycline-induced for three days (DIV3) to initiate neuronal differentiation and plated on 96-well PDL and laminin coated plates at 30 k/well and grown in Neuronal Differentiation Media (NDM) consisting of DMEM/F12 (Life Technologies 11330-057), Neurobasal media (Gibco 15240062), antibiotics, supplements, growth factors and doxycycline in an incubator (37° C./5% CO2). Cells were half-fed every seven days, and on DIV21, RNAi agent was serially diluted in NDM, and cells were treated with RNAi by aspirating 75 μL and adding 75 μL of 2×RNAi concentration for a final of 1×RNAi according to dilutions. Cells were half-fed every seven days after treatment by removing half of media and adding back fresh NDM. Cell lysates were harvested at DIV35 (14 days after RNAi treatment) or DIV42 (21 days after RNAi treatment) and RT-qPCR was performed using TaqMan Fast Advanced Cells-to-C_{T} Kit (ThermoFisher, A35377) and to determine mRNA knock down using SARM1 probe as the gene of interest (ThermoFisher, Hs00240907_m1) and ACTb probe as the housekeeping gene (ThermoFisher, Hs99999903_m1).

### Results

Table 10 summarizes the percentage knockdown of SARM1 mRNA and IC50 of the SARM RNAi agents in human SH-SY5Y cells. The tested SARM1 RNAi agents achieved robust SARM1 mRNA knockdown in human SH-SY5Y cells.

TABLE 10
In vitro activities of selected SARM1 RNAi agents

| | | | | hiPSC |
|---|---|---|---|---|
| | | SH-SY5Y, 3 d | | Neurons, 21 d |
| SARM1 | SH-SY5Y, | % KD | hiPSC | % KD |
| RNAi | 3 d | (knockdown) of | Neurons, 21 d | (knockdown) of |
| Agent | IC50 (nM) | SARM1 at 1 μM | IC50 (nM) | SARM1 at 1 μM |
| Conjugated | 99.41 | 71.91 | NA | NA |
| RNAi Agent | | | | |
| NO. 1 | | | | |
| Conjugated | 28.27 | 80.41 | NA | NA |
| RNAi Agent | | | | |
| NO. 2 | | | | |
| Conjugated | 23.7 | 77.28 | NA | NA |
| RNAi Agent | | | | |
| NO. 3 | | | | |
| Conjugated | 61.95 | 75.45 | NA | NA |
| RNAi Agent | | | | |
| NO. 4 | | | | |
| Conjugated | 53.11 | 75.68 | NA | NA |
| RNAi Agent | | | | |
| NO. 5 | | | | |
| Conjugated | 113.9 | 83.94 | NA | NA |
| RNAi Agent | | | | |
| NO. 6 | | | | |
| Conjugated | 6.30 | 82.25 | 13.05 | 70.37 |
| RNAi Agent | | | | |
| NO. 7 | | | | |
| Conjugated | 4.16 | 77.08 | 7.11 | 68.11 |
| RNAi Agent | | | | |
| NO. 8 | | | | |
| Conjugated | 4.63 | 84.37 | 14.31 | 68.05 |
| RNAi Agent | | | | |
| NO. 9 | | | | |
| Conjugated | 6.84 | 87.74 | 9.54 | 84 |
| RNAi Agent | | | | |
| NO. 10 | | | | |
| Conjugated | 5.16 | 83.84 | 8.86 | 67.96 |
| RNAi Agent | | | | |
| NO. 11 | | | | |
| Conjugated | 12.23 | 84.51 | 26.29 | 68.15 |
| RNAi Agent | | | | |
| NO. 12 | | | | |

“NA” means not available.

### Example 4. Knockdown of SARM1 mRNA in the Cynomolgus Monkey Following a Single Intrathecal Administration of Selected RNAi Agent

The efficacy of selected SAR 1 RNAi agents were tested in cynomolgus monkey (Macaca fascicularis). To assess the efficacy of the RNAi agent in knocking down SARM1; five groups of four cynomolgus monkeys per group were ported with indwelling catheters at L4/L5 with the catheter tip placed at T12/L1. The monkeys were infused with either aCSF (cerebrospinal fluid) or 20 mg of the Conjugated RNAi Agent No. 1-4 (8 mg/ml in aCSF) over 15 minutes and were perfused 28 days later. Tissues collected at necropsy included spinal cord (lumbar, cervical, thoracic) and brain (motor cortex, medulla, pons, and midbrain). qPCR was performed to determine mRNA knockdown in multiple spinal cord and brain regions.

Table 11 summarizes the percentage knockdown of SARM1 mRNA in cynomolgus monkey. As shown in Table 11, robust SARM1 mRNA knockdown was observed in all the tested regions 28 days after a single intrathecal administration of the RNAi agent.

TABLE 11
The Percentage Knockdown (KD) of SARM1 mRNA in Cynomolgus Monkey following
a single intrathecal administration of selected RNAi Agent.

| Conjugated | % KD | % KD | % KD | | | | |
|---|---|---|---|---|---|---|---|
| RNAi | Lumbar | Cervical | Thoracic | % KD | | | |
| Agent | Spinal | Spinal | Spinal | Motor | % KD | % KD | % KD |
| No. | Cord | Cord | Cord | Cortex | Medulla | Pons | Midbrain |
| 1 | 76.93 | 57.80 | 71.20 | 72.46 | 68.31 | 31.13 | 32.07 |
| 2 | 79.38 | 69.82 | 76.63 | 78.31 | 78.28 | 60.38 | 46.89 |
| 3 | 78.83 | 68.22 | 73.99 | 74.06 | 67.33 | 56.47 | 42.90 |
| 4 | 59.99 | 38.46 | 49.01 | 62.27 | 68.22 | 27.73 | 22.92 |

### Example 5. Knockdown of Human SARM1 mRNA in the Liver of AAV Mouse Model

All animals were individually housed in a temperature-controlled facility with 12 h/12 h light/dark cycle. Animal protocols in this study were approved by the Eli Lilly and Co., Animal Use and Care Committee (Protocol No. 20-025). A liver centric model over-expressing human SARM1 was generated using ˜8-week-old C57BL/6 male mice through a single dose administration of 10¹¹ genome copies (GC) of AAV8-TBG vector harboring Homo sapiens SARM1 in PBS by intravenous injection. At 14 days post-AAV administration, a selected RNAi agent or control agent (PBS) were subcutaneously injected at 3 mg/kg (n=6 per group). Mice were sacrificed and livers assessed for human SARM1 mRNA levels at 14 days post-subcutaneous injection of RNAi agent or control. Knockdown levels of in vivo human SARM mRNA were determined by comparison to control group.

Table 12 summarizes the percentage knockdown of human SARM1 mRNA in the liver of AAV mouse model.

TABLE 12
The Percentage Knockdown (KD) of human SARM1
mRNA in the liver of AAV mouse model.

| | % KD of human | |
|---|---|---|
| | SARM1 in the liver | Standard |
| Agent | of AAV mouse model | Error |
| Control | 0 | 0.1 |
| Conjugated RNAi Agent No. 13 | 47.9 | 3.9 |
| Conjugated RNAi Agent No. 14 | 60.3 | 3.9 |
| Conjugated RNAi Agent No. 15 | 59.1 | 3.2 |
| Conjugated RNAi Agent No. 16 | 64.5 | 5.8 |
| Conjugated RNAi Agent No. 17 | 36.2 | 15.3 |
| Conjugated RNAi Agent No. 18 | 9.4 | 15.3 |
| Conjugated RNAi Agent No. 19 | 29.6 | 5.9 |
| Conjugated RNAi Agent No. 20 | 47.5 | 5.7 |
| Conjugated RNAi Agent No. 21 | 47.4 | 1.8 |
| Conjugated RNAi Agent No. 22 | 40.7 | 5.5 |
| Conjugated RNAi Agent No. 23 | 24.9 | 4.5 |
| Conjugated RNAi Agent No. 24 | 57.9 | 3.4 |
| Conjugated RNAi Agent No. 25 | 58.6 | 2.9 |
| Conjugated RNAi Agent No. 26 | 55.6 | 3.4 |
| Conjugated RNAi Agent No. 27 | 32.9 | 2.8 |

SEQUENCE LISTING

| SEQ ID NO | Sequence | |
|---|---|---|
| 1 | GUUGCUCGACUCUAACCGCUA | |
| 2 | UAGCGGUUAGAGUCGAGCAACGG | |
| 3 | UUCGCCAACUAUUCUACGUGA | |
| 4 | UCACGUAGAAUAGUUGGCGAAGG | |
| 5 | ACCUUCGCCAACUAUUCUACA | |
| 6 | UGUAGAAUAGUUGGCGAAGGUCU | |
| 7 | CGCCAACUAUUCUACGUGCGA | |
| 8 | UCGCACGUAGAAUAGUUGGCGAA | |
| 9 | CCGCAAGAGGUUCUUUAGGGA | |
| 10 | UCCCUAAAGAACCUCUUGCGGGU | |
| 11 | AAACGCCUGGUUUCCUACUCA | |
| 12 | UGAGUAGGAAACCAGGCGUUUCA | |
| 13 | mG*mU*mUmGmCmUfCmGfAfCfUmCmUmAmAmCmCmGmC*mU*mA | |
| 14 | VPmU*fA*mGmCmGfGmUmUmAmGmAmGmUfCmGfAmGmCmAmAmC*mG*mG | |
| 15 | mU*mU*mCmGmCmCfAmAfCfUfAmUmUmCmUmAmCmGmU*mG*mA | |
| 16 | VPmU*fC*mAmCmGfUmAmGmAmAmUmAmGfUmUfGmGmCmGmAmA*mG*mG | |
| 17 | mA*mC*mCmUmUmCfGmCfCfAfAmCmUmAmUmUmCmUmA*mC*mA | |
| 18 | VPmU*fG*mUmAmGfAmAmUmAmGmUmUmGfGmCfGmAmAmGmGmU*mC*mU | |
| 19 | mC*mG*mCmCmAmAfCmUfAfUfUmCmUmAmCmGmUmGmC*mG*mA | |
| 20 | VPmU*fC*mGmCmAfCmGmUmAmGmAmAmUfAmGfUmUmGmGmCmG*mA*mA | |
| 21 | mC*mC*mGmCmAmAfGmAfGfGfUmUmCmUmUmUmAmGmG*mG*mA | |
| 22 | VPmU*fC*mCmCmUfAmAmAmGmAmAmCmCfUmCfUmUmGmCmGmG*mG*mU | |
| 23 | mA*mA*mAmCmGmCfCmUfGfGfUmUmUmCmCmUmAmCmU*mC*mA | |
| 24 | VPmU*fG*mAmGmUfAmGmGmAmAmAmCmCfAmGfGmCmGmUmUmU*mC*mA | |
| 25 | 1 | ATCTCCCAGC TCAGCCGAGC CCGTGCCCAG GCCACGCTTT GTTCCAGCCG CCGCCTCCTC |
| | 61 | TACCCTACGG CGTCCGGAGC CATCCCTCGC CTGCTCGCTC TCTCCTTTCG CCCACTCCCT |
| | 121 | GCATCTGGGC CTGCATCACC TTTGCCAACC GCTCCCCCGA TCCTGCCGAC ACTCCTCCCC |
| | 181 | CAAACTTCTG ACCGGCACCC TTGCCTGGTA CCCTTCTCTC CATTCCTCCC CCTCCATCTT |
| | 241 | CTTTCCCCGA CCCCTCTCGG GTCCCTCTTT TCCCAAAACC CGGGTCTCTC CGCGTGGCCC |
| | 301 | CGCCTCCAGG CCGGGGATGT CCCCCGCGGC CCCGCGCCCA TGGTCCTGAC GCTGCTTCTC |
| | 361 | TCCGCCTACA AGCTGTGTCG CTTCTTCGCC ATGTCGGGCC CACGGCCGGG CGCCGAGCGG |
| | 421 | CTGGCGGTGC CTGGGCCAGA TGGGGGCGGT GGCACGGGCC CATGGTGGGC TGCGGGTGGC |
| | 481 | CGCGGGCCCC GCGAAGTGTC GCCGGGGGCA GGCACCGAGG TGCAGGACGC CCTGGAGCGC |
| | 541 | GCGCTGCCGG AGCTGCAGCA GGCCTTGTCC GCGCTGAAGC AGGCGGGGGG CGCGCGGGCC |
| | 601 | GTGGGCGCCG GCCTGGCCGA GGTCTTCCAA CTGGTGGAGG AGGCCTGGCT GCTGCCGGCC |
| | 661 | GTGGGCCGCG AGGTAGCCCA GGGTCTGTGC GACGCCATCC GCCTCGATGG CGGCCTCGAC |
| | 721 | CTGCTGTTGC GGCTGCTGCA GGCGCCGGAG TTGGAGACGC GTGTGCAGGC CGCGCGCCTG |
| | 781 | CTGGAGCAGA TCCTGGTGGC TGAGAACCGA GACCGCGTGG CGCGCATTGG GCTGGGCGTG |
| | 841 | ATCCTGAACC TGGCGAAGGA ACGCGAACCC GTAGAGCTGG CGCGGAGCGT GGCAGGCATC |
| | 901 | TTGGAGCACA TGTTCAAGCA TTCGGAGGAG ACATGCCAGA GGCTGGTGGC GGCCGGCGGC |
| | 961 | CTGGACGCGG TGCTGTATTG GTGCCGCCGC ACGGACCCCG CGCTGCTGCG CCACTGCGCG |
| | 1021 | CTGGCGCTGG GCAACTGCGC GCTGCACGGG GGCCAGGCGG TGCAGCGACG CATGGTAGAG |
| | 1081 | AAGCGCGCAG CCGAGTGGCT CTTCCCGCTC GCCTTCTCCA AGGAGGACGA GCTGCTTCGG |
| | 1141 | CTGCACGCCT GCCTCGCAGT AGCGGTGTTG GCGACTAACA AGGAGGTGGA GCGCGAGGTG |
| | 1201 | GAGCGCTCGG GCACGCTGGC GCTCGTGGAG CCGCTTGTGG CCTCGCTGGA CCCTGGCCGC |
| | 1261 | TTCGCCCGCT GTCTGGTGGA CGCCAGCGAC ACAAGCCAGG GCCGCGGGCC CGACGACCTG |
| | 1321 | CAGCGCCTCG TGCCGTTGCT CGACTCTAAC CGCTTGGAGG CGCAGTGCAT CGGGGCTTTC |
| | 1381 | TACCTCTGCG CCGAGGCTGC CATCAAGAGC CTGCAAGGCA AGACCAAGGT GTTCAGCGAC |
| | 1441 | ATCGGCGCCA TCCAGAGCCT GAAACGCCTG GTTTCCTACT CTACCAATGG CACTAAGTCG |
| | 1501 | GCGCTGGCCA AGCGCGCGCT GCGCCTGCTG GGCGAGGAGG TGCCACGGCC CATCCTGCCC |
| | 1561 | TCCGTGCCCA GCTGGAAGGA GGCCGAGGTT CAGACGTGGC TGCAGCAGAT CGGTTTCTCC |
| | 1621 | AAGTACTGCG AGAGCTTCCG GGAGCAGCAG GTGGATGGCG ACCTGCTTCT GCGGCTCACG |
| | 1681 | GAGGAGGAAC TCCAGACCGA CCTGGGCATG AAATCGGGCA TCACCCGCAA GAGGTTCTTT |
| | 1741 | AGGGAGCTCA CGGAGCTCAA GACCTTCGCC AACTATTCTA CGTGCGACCG CAGCAACCTG |
| | 1801 | GCGGACTGGC TGGGCAGCCT GGACCCGCGC TTCCGCCAGT ACACCTACGG CCTGGTCAGC |
| | 1861 | TGCGGCCTGG ACCGCTCCCT GCTGCACCGC GTGTCTGAGC AGCAGCTGCT GGAAGACTGC |
| | 1921 | GGCATCCACC TGGGCGTGCA CCGCGCCCGC ATCCTCACGG CGGCCAGAGA AATGCTACAC |
| | 1981 | TCCCCGCTGC CCTGTACTGG TGGCAAACCC AGTGGGGACA CTCCAGATGT CTTCATCAGC |
| | 2041 | TACCGCCGGA ACTCAGGTTC CCAGCTGGCC AGTCTCCTGA AGGTGCACCT GCAGCTGCAT |
| | 2101 | GGCTTCAGTG TCTTCATTGA TGTGGAGAAG CTGGAAGCAG GCAAGTTCGA GGACAAACTC |
| | 2161 | ATCCAGAGTG TCATGGGTGC CCGCAACTTT GTGTTGGTGC TATCACCTGG AGCACTGGAC |
| | 2221 | AAGTGCATGC AAGACCATGA CTGCAAGGAT TGGGTGCATA AGGAGATTGT GACTGCTTTA |
| | 2281 | AGCTGCGGCA AGAACATTGT GCCCATCATT GATGGCTTCG AGTGGCCTGA GCCCCAGGTC |
| | 2341 | CTGCCTGAGG ACATGCAGGC TGTGCTTACT TTCAACGGTA TCAAGTGGTC CCACGAATAC |
| | 2401 | CAGGAGGCCA CCATTGAGAA GATCATCCGC TTCCTGCAGG GCCGCTCCTC CCGGGACTCA |
| | 2461 | TCTGCAGGCT CTGACACCAG TTTGGAGGGT GCTGCACCCA TGGGTCCAAC CTAACCAGTC |
| | 2521 | CCCAGTTCCC CAGCCCTGCT GTGACTTCCA TTTCCATCGT CCTTTCTGAA GGAACAGCTC |
| | 2581 | CTGAAACCAG TCTCCCTGGG CTGAGACAAC CTGGGCTCTT CTTAGGAAAT GGCTCTCCCT |
| | 2641 | CCCCCTGTCC CCCACCCTCA TGGCCCACCT CCAACCCACT TTCCTCAGTA TCTGGAGAGG |
| | 2701 | GAAGGGAAGT CAGGCTTGGG CACGGGAGGT TAGAACTCCC CCAGGCCCTG CCATTGGGTT |
| | 2761 | GTCTGTCTCC GTCATGGGGA GGGTCCCTGC TCAGTTCTGG AGACACTGGA GTTGGGGTGG |
| | 2821 | GGGTGGTTCT GCATTCCCTT CTCCTGCTGA TAGCAGTCAG CTTGAGGAGG ATGACGGAAG |
| | 2881 | GCAGCCTCAG ACAGGAATTA AGGCAATGCC CAGGCGGGCC TGGGCACTGT ATTCTGAGCA |
| | 2941 | AGGGCCTGGG CCCAGGAGCC AGCCAGGGAT GAGTGCCATC ATGGCTCTCC ACTCAGACTG |
| | 3001 | TGCCTGGCCC CTGCACTTAC AACTTCCTGC CGCTCTGTGG CCTTGCCCTG TAATCACTCA |
| | 3061 | GTGCCCTTAG CTAGCCTGAC TAAGTCCCAG ATCCCCTACA GCTTCCTTCG GTGTGGTATC |
| | 3121 | TTTTGCCACA TCCAGGGCGA GGGTTGAGGC AAACCAGCCC TCCCTCTGAC TTCCTTGTCA |
| | 3181 | CTGCAGCCAG CTTTGCTGCA CTTGCTGGTG CACAGGAGCC TCCTGTTTGG GCCTGGGTCT |
| | 3241 | GGGCATGGGG AGGCCGTGCC TCAAAGCCCA CCCTACCCCA TGCCTTGGTG CTGTGCCTCA |
| | 3301 | GGCTCCTTCC TGGTCTGGCC CAGCTGGCTT CCCCAGCCCC TCAGCCATCC AGGGCTACCC |
| | 3361 | ACTGCTTACT CAGGGACCAG GCAGCCCCCA TGGCAGTAAA AGCAGCCTAG ACAGAACCTG |
| | 3421 | CAGCTCTGTG GAAAGAGGCA AAGTCCTGAA AAGGCAAAGG GTTGTCACTT AGGGCAGCTT |
| | 3481 | CTCCAACTTT AACATGCATC CAAGTCACCT GGGAATGTTG TTAAAATCAG GAGATCTGGG |
| | 3541 | GTGGGGCCTA GGACTCTGCA TTTCTTACAG ATTCCCAGGT GAGCTGATGC TGGTGGTTAA |
| | 3601 | GGGTAGCAAA TCTCTAAAGC ACGAAGCCCT CACAAATCTT TGCCATTTCC CAAACACTCC |
| | 3661 | GCTCCATGGT CTCCAGTCAT CAGAGCAACT CTACCTGGTA TTATCATCCC CATTTTACAG |
| | 3721 | ATAATGACAC TGAGGCTCAG AAAGGTTGAG GATAAGCCCA CTTTCCTGTC ATTAGTGGCA |
| | 3781 | GCCCCAGATC CAGACCTAGG CCTCCTGGCA CCCAGTCCAC TGGCAGTGGA ATTGCTTTCC |
| | 3841 | TGAGAATCAT TCTGAGGCTG GGCTATTGCT TCTCCCTTGC TTCAAAGAAT CTAGCAGCGG |
| | 3901 | GGGATAGGAT TTTGCAACAA AAAGCTGACC CAGAGGCCAT ACAGAGCAGG AATATCCCAT |
| | 3961 | TGCCCCCTCC TCCACTGGGT TCAGAGGGTA AGAAAGCACC CTCCAATAAA CCCAGGCTCC |
| | 4021 | AGGCCGTGGG GGCTGCTGAA GGCTCTTTCC CCGCAAGGGC CAGGTGTTGA CACCTTAAAG |
| | 4081 | CTGGCTGCGC CCCCAGCCCC ACTCTTGGCT GTGCTGGCCA GGTGACTCCT AGTTCTTGGC |
| | 4141 | CACATCATCA GAAAGTCAAA GGTCTCACTC CAGGTTTGGG GCTCCTTCCT TCCACTCCCC |
| | 4201 | TCCCTGCCAG AGTCTGTCTT GGCCAGTGCC AGCCTCGATG CTTTGGTTTT GACCCCACCT |
| | 4261 | GATCCTCCTT TCCTCATGCA GCACAAGTGC TCACCGGGGC CAGAGCCAGG GCATGGATAT |
| | 4321 | GACAAGCAGG GCAGCCTGGA CACTGCCCTC ACAGGACAGC GCCAATAACA ATACAGTGTC |
| | 4381 | TGAGTATCTC CAGGGGATGA TTTCTGGCTC TTTGTCTCCA ATCAGTCCCA CTCCCTCCTG |
| | 4441 | AGGTCCCCAA GGGCAGTATT CAGAGAGGTT TCCTGCGTTT TATTTCTATT TGGTATACCC |
| | 4501 | TCCACTGTTG TCCACTGCCC TGTGTGGCCT TCTGGTTGAC CTCTGCCCGA TCTTCTGTCT |
| | 4561 | CTCTGAGGGA ATCAGAGTCC AGCATCCAGC CCCAGCTGGA ACAGCTGAAG TCACAAGCCT |
| | 4621 | CCTCTAAGCC AAGGCCAGTG TGTTCAGAGG TGACTGCCAC CCATACTAGG ACAAACACAG |
| | 4681 | CTCAGATCAC CAGGTCAAGC ACCTAGGCCT GGCTTCTCCT GAGACAGAGG ACTCAGAAGT |
| | 4741 | GGCCTTTCCT CCAAAGCCTG CTCAGACACA GGTCTGTAGG GCCAGGGTGT TCTGCTTGGC |
| | 4801 | TGGGCTGCAG CTGCTACCCC TCGGTTGGGG CTGAGTCAGC CAGATCCTCC CCCTACTTCT |
| | 4861 | CCCCAAGGGC CAAGAACTGC TCAGGGACAT TAAAGGTCAA AAGTCCAGCC ACACTCATTC |
| | 4921 | ATCCTTTCCC CAGGCCCATG AAGAGAGGCA TCTCATTGTA GAATGTATGA GGAAGTGGGA |
| | 4981 | AGTATCTCAG AGAATCAGCT AAGTTTCCTA ACTTGTCCAT CCAAATGTGA TCACCACGAT |
| | 5041 | TCAACAATTT GGGGCATTGC TGATCTAGCC GTTCCTAGTG GGGCTTGCTC AAGGTTGCAC |
| | 5101 | AGCGAGTCAG TAGAAGCCCT GGCTGGCCCC ACTTGGTACC AATCCACCAG GCAGCTCAGG |
| | 5161 | GCTCCTGCCC AGCCCAGCAG CTTCTGTTGT CTAACGTATG GCAGGCAGAC TGGGAGCAGG |
| | 5221 | AAAACAGAGG GCCCCAAAGC CCAAGGCACC AGAAGGTTTG TTTCAGTTTG CTGAAGCTGA |
| | 5281 | TTTGTAATGA TTGGCACTCT TCAGCCAGGG GAGTGGGTAG GCCATAGCCA AGGATCGATT |
| | 5341 | CCCCAACCAC AGCAAAGGCA ACACTCTTCC TCCAGAGATC ACCAAGCCCC TCTTACCTCC |
| | 5401 | CTCCCTCCTT CCCAAGGCTG GCACTAACCA GGTACCACAT TCATTGTTAA GGAATGGCTG |
| | 5461 | ATGACTGCTA CACGTGTTGG GAACCTGGTT GGGGCTGTGC AGTTTGGGCT GGAAGGAGAG |
| | 5521 | ATGCCAGCCC TCGTGCTGCC TCTGGTCCCT GAAGTGTCAC CTCTCTCAGG ACCTCTCCTC |
| | 5581 | TGGCCTGTGG GGTTATAAGT GATGGATAGC AGAAAGGGAG AACTGACTCC TGTCCCAAAT |
| | 5641 | AGCTCCTCTG CCACCTGTCC TGCAGTGGGC CTGTGTGGGT TATGATTCTA GATCCTAGAC |
| | 5701 | AGAGGCTGGG TCAGCTGTGG ATGGGGTGGT GCCTTGGTCT CTCTTGACTA CCTCGTCCAA |
| | 5761 | AGAGAGCACT GCCCTTAGAC AAGAGTTGCT TGTCCTGCTG TGGGCTGGGC TTCCAGCTGC |
| | 5821 | AGACCTCCAG TTGCTTGGTG TTCACTTTGC TCCTCTTGCC CTCTGTCTTC TGGTCCAGGC |
| | 5881 | AGATCAGGGG CTCTGGGGAA ACTGCTGGAA CTCGAGGTGA GGATCAGCCT TTTCCAGCAT |
| | 5941 | CCTGTGAGAG ACCAGAGAGA GAGTTTGGAT TTCATGTGGG GAACCCTCAA GGCCTGTCTG |
| | 6001 | GAGAAGTGAC ACAGGATTTA CTGGGGTGGG CTGGTCCAGG TAGCTCTCCT GAACCTCCTC |
| | 6061 | CTTCCCCAAG CTGAGAAGCT GAGAGCTGGA GGACAATATC CAGGGACATG GCTCTGGAAA |
| | 6121 | ATAACTTTTT TTTTTTTAAG AGACAGGGTC TTGCTCTGTT GTCCAGGCTG GAGGGCAGTG |
| | 6181 | ACATAATCAT AGCTCACTGT ACCCTTGAAC TCCTGGGCTC AAGTGATCCT CCTGCCTCAG |
| | 6241 | CCTCCTTAGT AGCTGGGACT ACCAGTGCAT ACCACCATGC CTGGGTGATT TTTTAAATTT |
| | 6301 | TTTATACAGA CAAGGTCTTG CTATGTTGCC CAGGCTGATC TTGAATTCCC GGGCTCAAGT |
| | 6361 | GGTCCTCCTG CCTCAGCCTC CCACAGGATC GGGATTACAG GCAAGAGCCT CCACGCCCGG |
| | 6421 | CCATGAAATA TAATTCTTAA TATCATACAG GAAAAAGTCA GCGGGTCAAG CTAGCCTGTG |
| | 6481 | GCCCAGCCAC AACTAGCTGA CAAAGCTTCC TGGCCTTCCC TTTAACACAG TTCTGCTGCC |
| | 6541 | ATAGTTCCAT CTATAAAATG GGAATGGAGG GAAATAGGGG AACTGGGAGA GAGAACACAG |
| | 6601 | CCTTGCCAAG CAGCAATGTT AGCCTGATCC TTCCTCCACC TAGCTCGCCA TCTCGCCCTT |
| | 6661 | GGAAAATGGC TCCTGGAGGA TTAGGCAGCC ATCTGCAAGG AGAGGGGCAA CCTGGGACAA |
| | 6721 | GACACCCAGA GGGTAAGGAT TCCAGGAATG AAGCTGCCAT TTCTGGTTGG GAGGAGAAGA |
| | 6781 | GGAAACTTTT AAGAGAAAGG GCTCCATTAT GAGCATGGGT TCAGGGCCCT GCATTACCCA |
| | 6841 | ATCAGAACAG CCGGGATGAG CAGGAGGCCA GCTCCCAGGA GGAAGGGGAA CCCCTTCATA |
| | 6901 | AAGTTCAGAG TGGCTGGGTA GAGTGAGTTG AAGATGCCGG AGGCCGTCAG CATGGCCAGG |
| | 6961 | CTATTCACAC AGGCCACAGC AGAAAAGAGA GCACCTGTGA AGAAATAAAT ACCATACTCT |
| | 7021 | GGAGTCCGAA AGGGCCATAT TCCAACTCTG GCACCACCAC CTCACAGCTG TGTGACCGGG |
| | 7081 | AGTAGTCACT TAACCTATGT CTCCCCTTCC TCACCAGTAA ATCCTGCTAC ATCATGTACT |
| | 7141 | GTGACAAGGA TTCAGTAAGG TCATATGTGG ACAGTAGCTG GCACAGAGGG GCTACTAAAC |
| | 7201 | AAATGGCTGC TATTAAATCC ACATTAAAAG TACATGTGAT CTGACAGAAC CCAGCACATA |
| | 7261 | AAAGAAAAAA AAAGTACATG TGATATTGTC TGATGAAAGC TTGATGGAAA TGGCTTTTTT |
| | 7321 | CTGGTTTATC CTCTTTGGAA TCATCTCCTG TTTGGGATTA ACTGCTGGTC TGATCAGTTC |
| | 7381 | CAATATTCAT AGCGGTGTCA CCACTGAATA GCTTCTTATC CTTTGGGTTC CTGTTCCTCC |
| | 7441 | TTCTGCTAAA TAAGGATAAT ACCTATTTCC TAGATTGTGA GCAACATTAA GTTCACATGG |
| | 7501 | AAATCACCCA TCACTGGGCC TGGTCCCCTG GAAGTAGCTA GTTAGTAAGG GCTGTTCTTT |
| | 7561 | TCTCCTGTTT CTCTTGACAT CTCTGGGCAC AGAGAAAGTG CTGGGAAAAA AAGTTTAGGT |
| | 7621 | GAATGAATGA AGACACATGG ATTCTGGGGA CACCAGAACC CACAGTGGGC TCTGTATGGC |
| | 7681 | ACCAGAGTCT CTGTCATCAT CAGATCCTCA TTCCAGGACA GATGGAAAAA GATGAATGTT |
| | 7741 | TCCAGACTGG GGCATAAAGA CCCAGAGGCT GGAGAAGCTG TTCTTTATAG ATATACCAGG |
| | 7801 | AGAACCCACA GTTTACAAAA TGTGCAACAA CCCAACAGAA GTTGAGATTA AATTCTGTCA |
| | 7861 | CATCTAGAGG GGTCTGTGAT GTCATCAAAA GCAAACCACC CACATCACAG ATGAAGAAAC |
| | 7921 | AGGCCTGTGG CAGGGCTCGG ACTAAAACCC AGATCCTGAG ACCAGCTGCT TTTAAACACA |
| | 7981 | GACGTAGGTT TGCATCCTAG CTCCACCATT TACTGAGTAA CCTTGGGTGA GCCAATGTAA |
| | 8041 | CCCCCTGGGT CTCTGTTTCT TTATCTGTCA ACTGTGGAAA ATGAAACCCA TGTCACAAGG |
| | 8101 | TTGTTCACTT CTGGGCTTGT ACACGCTGAC CCCAGAGAAA CAGGGAACTC TGGCATCACC |
| | 8161 | ACACCCATCT TACAGACGGA AAAGCTGAGG TCTGCAGAGA GTAAATCCTC TGCTCTGGTT |
| | 8221 | ATCTAGAAAG AACATAATTG TGCTCTGCTG ACTGCAAATC CCAACTCTGC GGTTTGAAAA |
| | 8281 | TCCAAGGTGG CATGATCCTC TGCCCATTGT GGGCAATTTC ACAGAAATGT GTTTGTTTTG |
| | 8341 | GCCACTTACT TCTCCAGGGT GAGAGGGGGG AAGGCAAGCT GTTCCCCCAG CCATGGCTGC |
| | 8401 | CCATCAGCCC GTTTCGGGCA GCACTGGACA TGAGGAACCA GACACAGGTG GGTTCTGACA |
| | 8461 | CTCACCCTGC TCTGTCTCTC TCACCAGCTT GGAGAGTTTA GCCCGGATGA CAGGTGTGAT |
| | 8521 | GACTAATGAC AGGAAAAGCA ACCCATATCC TGTGGAGAAA CAAACACTCA TCAGGAAAAT |
| | 8581 | GGGGCTGGGG AGAGGGGCGT CCAAGGGAAA GGCAGCAGAG CTCCTATCCA TACCCCACGT |
| | 8641 | GGGGCTTAGG TTAGACCCAG GAAGAACTTC CTTGATGGTG AGGGTGGGAA GACAGTAGTC |
| | 8701 | AAGGAGGAAT GGAGACTGCC CTTGTCTGGG CTTGGCCACC TGCTAGCTCT CATGAATGAA |
| | 8761 | TGCTAATTCC CATTGATTGC TTTCTTGTCT GAACCTCTTG TGGTCACAGC AGGCATCACC |
| | 8821 | CACCCACTTG GCACTTAGTA GGGATATGGC AGGGCACAGA AAACAAGCAT GGGCTTTGGA |
| | 8881 | GTCAGCCCTG AGTTCAAAAC CTGATGCCAT TACATATTAT CTGTGTGGCC TGGGGTACTT |
| | 8941 | ACCCTCTCTG ATCCTGACTC CCTGTATGAG GAAGATAATA AGGCCTTCAT CACAGGATGG |
| | 9001 | TTCTGAGGCA TAGGAGGCTG AATAATGGTG CCCAATGGCA TCAGATTCAT AGCCCTGGAA |
| | 9061 | CCTGTAAATA CTACCTTATT TGGAAAATGA GTCTATGCAG GTGTGCAGTT AAGCCTCCTG |
| | 9121 | AGAGAGCAGA GTTATCCTGG ATTAGGTTGG GCCCTAAATG CCGTCACACA TATCTTTATA |
| | 9181 | AGAGGAAAGC AGACGGAGAT TTGGCACCGA CAGAATTGAG AAGGCACAAA GAGGAGGAGA |
| | 9241 | GTCAATGTGA GCACAGAGGC AGAGACTGGT GATGGCCGCC CCAAGCCAAG GAATGCCAGC |
| | 9301 | AGCCCCAGAA GCTGGAAGAA ATGAGAAACA CGTTCTCTCC TGGAGGCTTG CAAGGGAGCA |
| | 9361 | CTGCCTGCTG ACTGCTTCCA TTCAGCCCGG TGGTACTGAC TTTGGACTTC TGGCCTCCAG |
| | 9421 | AACTGTGAGA GAATATGTTT CTGTTGTGTT AAGCCCCCAA GTTTGTGGTA TGTCATTACA |
| | 9481 | GCAATCTCAG GGAACCAATA CATGAGGTAA AAAGGTAACA TCTATGAAGA GCATGGCATA |
| | 9541 | GGGACACAGC AAATGGGAGT TCCTTTTCCC TTTGCATTCA GTTACTTACA GGCTTCCTGT |
| | 9601 | TTTCTTCATA ACCATTTCTC TCCCTGTGCG ACTGCTGACT CCTCAGCAAA ACTGCAAACT |
| | 9661 | CCTACAGGAC AGTGGATCCT CCAAAGAAGG TATACGATGA GGCATCCAGG GACCCTAGCA |
| | 9721 | GTGTCAGGCC CCTCAAATCC CACTCTGTTG AGACCTCCCC CCGACCCAGA GCAATGACAG |
| | 9781 | CATCTTTATC ATCTCTGCAT CCCCCAGGGC CATCAGCAGG AGGGAAAGGT TCCCTTCTGC |
| | 9841 | TTAATTGTCA GACAAGCAGT TGAGTTAAGA AATCTGTGAT TATTGTATTG TTGACTATAC |
| | 9901 | ACAGCACATT TTAGGGCTCT ATCAAAATAA ATCTGTCCCT TTAAAAAAAG TTAACTAAAG |
| | 9961 | CCGGGCACGG TGGCTCATGC CTGTAATCCC AACACTTTGG GAGGCTGAGG CAGGCGGATC |
| | 10021 | CTTGAGCTCA GGAGTTAGAG ACCTGGACTG GGCAAAATGG TGAGGACCCC ATCTCTATAA |
| | 10081 | AAAATACAAA AATTAGCAAG GTGTGGTAAT GTGCACCAGT GGTCCCAGCT ACTAGAGAGG |
| | 10141 | CCAAGGTGGG AGGATCATCT GGGCCCGGGG GATGAGGCTG CAGTGAGCCA TGATCGTGCC |
| | 10201 | ACTGCACTCT AGCCTGGGTA ACAAAGCGAG ACCCTGTCTC TAAATACATC AATCAAATAA |
| | 10261 | AAATTTTAAA AAGTTAA |
| 26 | 1 | MVLTLLLSAY KLCRFFAMSG PRPGAERLAV PGPDGGGGTG PWWAAGGRGP REVSPGAGTE |
| | 61 | VQDALERALP ELQQALSALK QAGGARAVGA GLAEVFQLVE EAWLLPAVGR EVAQGLCDAI |
| | 121 | RLDGGLDLLL RLLQAPELET RVQAARLLEQ ILVAENRDRV ARIGLGVILN LAKEREPVEL |
| | 181 | ARSVAGILEH MFKHSEETCQ RLVAAGGLDA VLYWCRRTDP ALLRHCALAL GNCALHGGQA |
| | 241 | VQRRMVEKRA AEWLFPLAFS KEDELLRLHA CLAVAVLATN KEVEREVERS GTLALVEPLV |
| | 301 | ASLDPGRFAR CLVDASDTSQ GRGPDDLQRL VPLLDSNRLE AQCIGAFYLC AEAAIKSLQG |
| | 361 | KTKVFSDIGA IQSLKRLVSY STNGTKSALA KRALRLLGEE VPRPILPSVP SWKEAEVQTW |
| | 421 | LQQIGFSKYC ESFREQQVDG DLLLRLTEEE LQTDLGMKSG ITRKRFFREL TELKTFANYS |
| | 481 | TCDRSNLADW LGSLDPRFRQ YTYGLVSCGL DRSLLHRVSE QQLLEDCGIH LGVHRARILT |
| | 541 | REMLHSPL PCTGGKPSGD TPDVFISYRR NSGSQLASLL KVHLQLHGFS VFIDVEKLEA |
| | 601 | FEDKLIQS VMGARNFVLV LSPGALDKCM QDHDCKDWVH KEIVTALSCG KNIVPIIDGF |
| | 661 | PEPQVLPE DMQAVLTFNG IKWSHEYQEA TIEKIIRFLQ GRSSRDSSAG SDTSLEGAAP |
| | 721 | MGPT |

| | |
|---|---|
| 27 | mU*fC*mAmCmGfUmAmGmAmAmUmAmGfUmUfGmGmCmGmAmA*mG*mG |
| 28 | mU*mU*mCmGmCmCmAmAfCfUfAmUmUmCmUmAmCmGmU*mG*mA |
| 29 | mU*fC*mAmCfGmUfAmGmAmAmUmAmGfUmUfGmGmCmGmAmA*mG*mG |
| 30 | mU*fC*fAmCmGmUfAmGmAmAmUmAmGfUmUfGmGmCmGmAmA*mG*mG |
| 31 | mU*fC*mAmCfGmUmAfGmAmAmUmAmGfUmUfGmGmCmGmAmA*mG*mG |
| 32 | mU*fC*mAmCmGmUmAmGmAmAmUmAmGfUmUmGmGmCmGmAmA*mG*mG |
| 33 | mU*fA*mGmCmGfGmUmUmAmGmAmGmUfCmGfAmGmCmAmAmC*mG*mG |
| 34 | mG*mU*mUmGmCmUmCmGfAfCfUmCmUmAmAmCmCmGmC*mU*mA |
| 35 | mU*fA*mGmCfGmGfUmUmAmGmAmGmUfCmGfAmGmCmAmAmC*mG*mG |
| 36 | mU*fA*fGmCmGmGfUmUmAmGmAmGmUfCmGfAmGmCmAmAmC*mG*mG |
| 37 | mU*fA*mGmCfGmGmUfUmAmGmAmGmUfCmGfAmGmCmAmAmC*mG*mG |
| 38 | mU*fA*mGmCmGmGmUmUmAmGmAmGmUfCmGmAmGmCmAmAmC*mG*mG |
| 39 | mU*fC*mCmCmUfAmAmAmGmAmAmCmCfUmCfUmUmGmCmGmG*mG*mU |
| 40 | mC*mC*mGmCmAmAmGmAfGfGfUmUmCmUmUmUmAmGmG*mG*mA |
| 41 | mU*fC*mCmCfUmAfAmAmGmAmAmCmCfUmCfUmUmGmCmGmG*mG*mU |
| 42 | mU*fC*fCmCmUmAfAmAmGmAmAmCmCfUmCfUmUmGmCmGmG*mG*mU |
| 43 | mU*fC*mCmCfUmAmAfAmGmAmAmCmCfUmCfUmUmGmCmGmG*mG*mU |
| 44 | mU*fC*mCmCmUmAmAmAmGmAmAmCmCfUmCmUmUmGmCmGmG*mG*mU |
| 45 | mU*fG*mUmAmGfAmAmUmAmGmUmUmGfGmCfGmAmAmGmGmU*mC*mU |
| 46 | mU*fC*mGmCmAfCmGmUmAmGmAmAmUfAmGfUmUmGmGmCmG*mA*mA |
| 47 | mU*fG*mAmGmUfAmGmGmAmAmAmCmCfAmGfGmCmGmUmUmU*mC*mA |

1. A SARM1 RNAi agent having a sense strand and an antisense strand, wherein the sense strand and the antisense strand form a duplex,
wherein the sense strand and the antisense strand comprise a pair of nucleic acid sequences selected from the group consisting of:
(a) the sense strand comprises a first nucleic acid sequence having at least 95% sequence identity to SEQ ID NO: 1, and the antisense strand comprises a second nucleic acid sequence having at least 95% sequence identity to SEQ ID NO: 2;
(b) the sense strand comprises a first nucleic acid sequence having at least 95% sequence identity to SEQ ID NO: 3, and the antisense strand comprises a second nucleic acid sequence having at least 95% sequence identity to SEQ ID NO: 4;
(c) the sense strand comprises a first nucleic acid sequence having at least 95% sequence identity to SEQ ID NO: 5, and the antisense strand comprises a second nucleic acid sequence having at least 95% sequence identity to SEQ ID NO: 6;
(d) the sense strand comprises a first nucleic acid sequence having at least 95% sequence identity to SEQ ID NO: 7, and the antisense strand comprises a second nucleic acid sequence having at least 95% sequence identity to SEQ ID NO: 8;
(e) the sense strand comprises a first nucleic acid sequence having at least 95% sequence identity to SEQ ID NO: 9, and the antisense strand comprises a second nucleic acid sequence having at least 95% sequence identity to SEQ ID NO: 10; and
(f) the sense strand comprises a first nucleic acid sequence having at least 95% sequence identity to SEQ ID NO: 11, and the antisense strand comprises a second nucleic acid sequence having at least 95% sequence identity to SEQ ID NO: 12,
wherein optionally one or more nucleotides of the sense strand and the antisense strand are independently modified nucleotides, and
wherein optionally one or more internucleotide linkages of the sense strand and the antisense strand are modified internucleotide linkages.

2. The SARM1 RNAi agent of claim 1, wherein the sense strand and the antisense strand comprise a pair of nucleic acid sequences selected from the group consisting of:
(a) the sense strand comprises a first nucleic acid sequence of SEQ ID NO: 1, and the antisense strand comprises a second nucleic acid sequence of SEQ ID NO: 2;
(b) the sense strand comprises a first nucleic acid sequence of SEQ ID NO: 3, and the antisense strand comprises a second nucleic acid sequence of SEQ ID NO: 4;
(c) the sense strand comprises a first nucleic acid sequence of SEQ ID NO: 5, and the antisense strand comprises a second nucleic acid sequence of SEQ ID NO: 6;
(d) the sense strand comprises a first nucleic acid sequence of SEQ ID NO: 7, and the antisense strand comprises a second nucleic acid sequence of SEQ ID NO: 8;
(e) the sense strand comprises a first nucleic acid sequence of SEQ ID NO: 9, and the antisense strand comprises a second nucleic acid sequence of SEQ ID NO: 10; and
(f) the sense strand comprises a first nucleic acid sequence of SEQ ID NO: 11, and the antisense strand comprises a second nucleic acid sequence of SEQ ID NO: 12.

3. The SARM1 RNAi agent of claim 1, wherein the sense strand and the antisense strand have a pair of nucleic acid sequences selected from the group consisting of:
(a) the sense strand has a first nucleic acid sequence of SEQ ID NO: 1, and the antisense strand has a second nucleic acid sequence of SEQ ID NO: 2;
(b) the sense strand has a first nucleic acid sequence of SEQ ID NO: 3, and the antisense strand has a second nucleic acid sequence of SEQ ID NO: 4;
(c) the sense strand has a first nucleic acid sequence of SEQ ID NO: 5, and the antisense strand has a second nucleic acid sequence of SEQ ID NO: 6;
(d) the sense strand has a first nucleic acid sequence of SEQ ID NO: 7, and the antisense strand has a second nucleic acid sequence of SEQ ID NO: 8;
(e) the sense strand has a first nucleic acid sequence of SEQ ID NO: 9, and the antisense strand has a second nucleic acid sequence of SEQ ID NO: 10; and
(f) the sense strand has a first nucleic acid sequence of SEQ ID NO: 11, and the antisense strand has a second nucleic acid sequence of SEQ ID NO: 12.

4. The SARM1 RNAi agent of claim 1, wherein one or more nucleotides of the sense strand are modified nucleotides.

5. The SARM1 RNAi agent of claim 1, wherein each nucleotide of the sense strand is a modified nucleotide.

6. The SARM1 RNAi agent of claim 1, wherein one or more nucleotides of the antisense strand are modified nucleotides.

7. The SARM1 RNAi agent of claim 1, wherein each nucleotide of the antisense strand is a modified nucleotide.

8. The SARM1 RNAi agent of claim 1, wherein the modified nucleotide is a 2′-fluoro modified nucleotide, 2′-O-methyl modified nucleotide or 2′-O-alkyl modified nucleotide.

9. The SARM1 RNAi agent of claim 8, wherein the sense strand has four 2′-fluoro modified nucleotides at positions 7, 9, 10, and 11 from the 5′ end of the sense strand.

10. The SARM1 RNAi agent of claim 9, wherein nucleotides at positions other than positions 7, 9, 10, and 11 of the sense strand are 2′-O-methyl modified nucleotides.

11. The SARM1 RNAi agent of claim 8, wherein the antisense strand has four 2′-fluoro modified nucleotides at positions 2, 6, 14, and 16 from the 5′ end of the antisense strand.

12. The SARM1 RNAi agent of claim 11, wherein nucleotides at positions other than positions 2, 6, 14 and 16 of the antisense strand are 2′-O-methyl modified nucleotides.

13. The SARM1 RNAi agent of claim 8, wherein the sense strand has three 2′-fluoro modified nucleotides at positions 9, 10, and 11 from the 5′ end of the sense strand.

14. The SARM1 RNAi agent of claim 13, wherein nucleotides at positions other than positions 9, 10, and 11 of the sense strand are 2′-O-methyl modified nucleotides.

15. The SARM1 RNAi agent of, claim 8, wherein the antisense strand has five 2′-fluoro modified nucleotides at positions 2, 5, 7, 14, and 16 from the 5′ end of the antisense strand.

16. The SARM1 RNAi agent of claim 15, wherein nucleotides at positions other than positions 2, 5, 7, 14, and 16 of the antisense strand are 2′-O-methyl modified nucleotides.

17. The SARM1 RNAi agent of, claim 8, wherein the antisense strand has five 2′-fluoro modified nucleotides at positions 2, 5, 8, 14, and 16 from the 5′ end of the antisense strand.

18. The SARM1 RNAi agent of claim 17, wherein nucleotides at positions other than positions 2, 5, 8, 14, and 16 of the antisense strand are 2′-O-methyl modified nucleotides.

19. The SARM1 RNAi agent of claim 8, wherein the antisense strand has five 2′-fluoro modified nucleotides at positions 2, 3, 7, 14, and 16 from the 5′ end of the antisense strand.

20. The SARM1 RNAi agent of claim 19, wherein nucleotides at positions other than positions 2, 3, 7, 14, and 16 of the antisense strand are 2′-O-methyl modified nucleotides.

21. The SARM1 RNAi agent of claim 8, wherein the antisense strand has two 2′-fluoro modified nucleotides at positions 2 and 14 from the 5′ end of the antisense strand.

22. The SARM1 RNAi agent of claim 21, wherein nucleotides at positions other than positions 2 and 14 of the antisense strand are 2′-O-methyl modified nucleotides.

23. The SARM1 RNAi agent of claim 8, wherein the sense strand and the antisense strand have one or more modified internucleotide linkages.

24. The SARM1 RNAi agent of claim 23, wherein the modified internucleotide linkage is phosphorothioate linkage.

25. The SARM1 RNAi agent of claim 24, wherein the sense strand has four or five phosphorothioate linkages.

26. The SARM1 RNAi agent of claim 24, wherein the antisense strand has four or five phosphorothioate linkages.

27. The SARM1 RNAi agent of claim 1, wherein the first nucleotide from the 5′ end of the antisense strand is a modified nucleotide that has a phosphate analog.

28. The SARM1 RNAi agent of claim 27, wherein the phosphate analog is 5′-vinylphosphonate.

29. The SARM1 RNAi agent of claim 1, wherein the sense strand comprises an abasic moiety or inverted abasic moiety.

30. A SARM1 RNAi agent having a sense strand and an antisense strand,
wherein the sense strand and the antisense strand form a duplex,
wherein the sense strand and the antisense strand comprise a pair of nucleic acid sequences selected from the group consisting of:
(a) the sense strand comprises a first nucleic acid sequence of SEQ ID NO: 13, and the antisense strand comprises a second nucleic acid sequence of SEQ ID NO: 14;
(b) the sense strand comprises a first nucleic acid sequence of SEQ ID NO: 15, and the antisense strand comprises a second nucleic acid sequence of SEQ ID NO: 16;
(c) the sense strand comprises a first nucleic acid sequence of SEQ ID NO: 17, and the antisense strand comprises a second nucleic acid sequence of SEQ ID NO: 18;
(d) the sense strand comprises a first nucleic acid sequence of SEQ ID NO: 19, and the antisense strand comprises a second nucleic acid sequence of SEQ ID NO: 20;
(e) the sense strand comprises a first nucleic acid sequence of SEQ ID NO: 21, and the antisense strand comprises a second nucleic acid sequence of SEQ ID NO: 22;
(f) the sense strand comprises a first nucleic acid sequence of SEQ ID NO: 23, and the antisense strand comprises a second nucleic acid sequence of SEQ ID NO: 24;
(g) the sense strand comprises a first nucleic acid sequence of SEQ ID NO: 15, and the antisense strand comprises a second nucleic acid sequence of SEQ ID NO: 27;
(h) the sense strand comprises a first nucleic acid sequence of SEQ ID NO: 28, and the antisense strand comprises a second nucleic acid sequence of SEQ ID NO: 29;
(i) the sense strand comprises a first nucleic acid sequence of SEQ ID NO: 28, and the antisense strand comprises a second nucleic acid sequence of SEQ ID NO: 30;
(j) the sense strand comprises a first nucleic acid sequence of SEQ ID NO: 28, and the antisense strand comprises a second nucleic acid sequence of SEQ ID NO: 31;
(k) the sense strand comprises a first nucleic acid sequence of SEQ ID NO: 28, and the antisense strand comprises a second nucleic acid sequence of SEQ ID NO: 32;
(l) the sense strand comprises a first nucleic acid sequence of SEQ ID NO: 13, and the antisense strand comprises a second nucleic acid sequence of SEQ ID NO: 33;
(m) the sense strand comprises a first nucleic acid sequence of SEQ ID NO: 34, and the antisense strand comprises a second nucleic acid sequence of SEQ ID NO: 35;
(n) the sense strand comprises a first nucleic acid sequence of SEQ ID NO: 34, and the antisense strand comprises a second nucleic acid sequence of SEQ ID NO: 36;
(o) the sense strand comprises a first nucleic acid sequence of SEQ ID NO: 34, and the antisense strand comprises a second nucleic acid sequence of SEQ ID NO: 37;
(p) the sense strand comprises a first nucleic acid sequence of SEQ ID NO: 34, and the antisense strand comprises a second nucleic acid sequence of SEQ ID NO: 38;
(q) the sense strand comprises a first nucleic acid sequence of SEQ ID NO: 21, and the antisense strand comprises a second nucleic acid sequence of SEQ ID NO: 39;
(r) the sense strand comprises a first nucleic acid sequence of SEQ ID NO: 40, and the antisense strand comprises a second nucleic acid sequence of SEQ ID NO: 41;
(s) the sense strand comprises a first nucleic acid sequence of SEQ ID NO: 40, and the antisense strand comprises a second nucleic acid sequence of SEQ ID NO: 42;
(t) the sense strand comprises a first nucleic acid sequence of SEQ ID NO: 40, and the antisense strand comprises a second nucleic acid sequence of SEQ ID NO: 43;
(u) the sense strand comprises a first nucleic acid sequence of SEQ ID NO: 40, and the antisense strand comprises a second nucleic acid sequence of SEQ ID NO: 44;
(v) the sense strand comprises a first nucleic acid sequence of SEQ ID NO: 17, and the antisense strand comprises a second nucleic acid sequence of SEQ ID NO: 45;
(w) the sense strand comprises a first nucleic acid sequence of SEQ ID NO: 19, and the antisense strand comprises a second nucleic acid sequence of SEQ ID NO: 46; and
(x) the sense strand comprises a first nucleic acid sequence of SEQ ID NO: 23, and the antisense strand comprises a second nucleic acid sequence of SEQ ID NO: 47.

31. The SARM1 RNAi agent of claim 30, wherein the sense strand and the antisense strand have a pair of nucleic acid sequences selected from the group consisting of:
(a) the sense strand has a first nucleic acid sequence of SEQ ID NO: 13, and the antisense strand has a second nucleic acid sequence of SEQ ID NO: 14;
(b) the sense strand has a first nucleic acid sequence of SEQ ID NO: 15, and the antisense strand has a second nucleic acid sequence of SEQ ID NO: 16;
(c) the sense strand has a first nucleic acid sequence of SEQ ID NO: 17, and the antisense strand has a second nucleic acid sequence of SEQ ID NO: 18;
(d) the sense strand has a first nucleic acid sequence of SEQ ID NO: 19, and the antisense strand has a second nucleic acid sequence of SEQ ID NO: 20;
(e) the sense strand has a first nucleic acid sequence of SEQ ID NO: 21, and the antisense strand has a second nucleic acid sequence of SEQ ID NO: 22;
(f) the sense strand has a first nucleic acid sequence of SEQ ID NO: 23, and the antisense strand has a second nucleic acid sequence of SEQ ID NO: 24;
(g) the sense strand has a first nucleic acid sequence of SEQ ID NO: 15, and the antisense strand has a second nucleic acid sequence of SEQ ID NO: 27;
(h) the sense strand has a first nucleic acid sequence of SEQ ID NO: 28, and the antisense strand has a second nucleic acid sequence of SEQ ID NO: 29;
(i) the sense strand has a first nucleic acid sequence of SEQ ID NO: 28, and the antisense strand has a second nucleic acid sequence of SEQ ID NO: 30;
(j) the sense strand has a first nucleic acid sequence of SEQ ID NO: 28, and the antisense strand has a second nucleic acid sequence of SEQ ID NO: 31;
(k) the sense strand has a first nucleic acid sequence of SEQ ID NO: 28, and the antisense strand has a second nucleic acid sequence of SEQ ID NO: 32;
(l) the sense strand has a first nucleic acid sequence of SEQ ID NO: 13, and the antisense strand has a second nucleic acid sequence of SEQ ID NO: 33;
(m) the sense strand has a first nucleic acid sequence of SEQ ID NO: 34, and the antisense strand has a second nucleic acid sequence of SEQ ID NO: 35;
(n) the sense strand has a first nucleic acid sequence of SEQ ID NO: 34, and the antisense strand has a second nucleic acid sequence of SEQ ID NO: 36;
(o) the sense strand has a first nucleic acid sequence of SEQ ID NO: 34, and the antisense strand has a second nucleic acid sequence of SEQ ID NO: 37;
(p) the sense strand has a first nucleic acid sequence of SEQ ID NO: 34, and the antisense strand has a second nucleic acid sequence of SEQ ID NO: 38;
(q) the sense strand has a first nucleic acid sequence of SEQ ID NO: 21, and the antisense strand has a second nucleic acid sequence of SEQ ID NO: 39;
(r) the sense strand has a first nucleic acid sequence of SEQ ID NO: 40, and the antisense strand has a second nucleic acid sequence of SEQ ID NO: 41;
(s) the sense strand has a first nucleic acid sequence of SEQ ID NO: 40, and the antisense strand has a second nucleic acid sequence of SEQ ID NO: 42;
(t) the sense strand has a first nucleic acid sequence of SEQ ID NO: 40, and the antisense strand has a second nucleic acid sequence of SEQ ID NO: 43;
(u) the sense strand has a first nucleic acid sequence of SEQ ID NO: 40, and the antisense strand has a second nucleic acid sequence of SEQ ID NO: 44;
(v) the sense strand has a first nucleic acid sequence of SEQ ID NO: 17, and the antisense strand has a second nucleic acid sequence of SEQ ID NO: 45;
(w) the sense strand has a first nucleic acid sequence of SEQ ID NO: 19, and the antisense strand has a second nucleic acid sequence of SEQ ID NO: 46; and
(x) the sense strand has a first nucleic acid sequence of SEQ ID NO: 23, and the antisense strand has a second nucleic acid sequence of SEQ ID NO: 47.

32. The SARM1 RNAi agent of claim 1, wherein the sense strand has a delivery moiety conjugated to the 5′ or 3′ end of the sense strand.

33. (canceled)

34. The SARM1 RNAi agent of claim 1, wherein the sense strand has a delivery moiety conjugated to a nucleotide of the sense strand.

35. The SARM1 RNAi agent of claim 32, wherein the delivery moiety is conjugated to the 5′ or 3′ end of the sense stand via a linker.

36. A pharmaceutical composition comprising the SARM1 RNAi agent of claim 1 and a pharmaceutically acceptable carrier.

37. A pharmaceutical composition comprising a means for reducing SARM1 expression in a cell and a pharmaceutically acceptable carrier.

38. A method of reducing SARM1 expression in a patient in need thereof, the method comprising administering to the patient an effective amount of the SARM1 RNAi agent of claim 1.

39. A method of reducing axon degeneration in a patient in need thereof, the method comprising administering to the patient an effective amount of the SARM1 RNAi agent of claim 1.

40. A method of treating a SARM1-mediated neurological disease in a patient in need thereof, the method comprising administering to the patient an effective amount of the SARM1 RNAi agent of claim 1.

41. The method of claim 40, wherein the SARM1-mediated neurological disease is selected from amyotrophic lateral sclerosis (ALS, or Lou Gehrig's disease), Alzheimer's disease, Parkinson's disease, multiple sclerosis (MS), Huntington's disease (HD), senile dementia, Pick's disease, Gaucher's disease, Hurler syndrome, progressive multifocal leukoencephalopathy, Alexander's disease, congenital hypomyelination, encephalomyelitis, acute disseminated encephalomyelitis, central pontine myelinolysis, osmotic hyponatremia, Tay-Sachs disease, motor neuron disease, ataxia, spinal muscular atrophy (SMA), Niemann-Pick disease, acute hemorrhagic leukoencephalitis, trigeminal neuralgia, Bell's palsy, cerebral ischemia, multiple system atrophy, Pelizaeus Merzbacher disease, periventricular leukomalacia, a hereditary ataxia, noise-induced hearing loss, congenital hearing loss, age-related hearing loss, Creutzfeldt-Jakob disease, transmissible spongiform encephalopathy, Lewy Body Dementia, frontotemporal dementia, tauopathy, synucleinopathy, amyloidosis, diabetic neuropathy, globoid cell leukodystrophy (Krabbe's disease), Bassen-Komzweig syndrome, transverse myelitis, motor neuron disease, spinocerebellar ataxia, pre-eclampsia, hereditary spastic paraplegias, spastic paraparesis, familial spastic paraplegia, French settlement disease, Strumpell-Lorrain disease, non-alcoholic steatohepatitis (NASH), adrenomyeloneuropathy, progressive supra nuclear palsy (PSP), Friedrich's ataxia, spinal cord injury, acute optic neuropathy (AON), a genetic or idiopathic retinal condition, Leber congenital amaurosis (LCA), Leber hereditary optic neuropathy (LHON), primary open-angle glaucoma (POAG), acute angle-closure glaucoma (AACG), autosomal dominant optic atrophy, retinal ganglion degeneration, retinitis pigmentosa, an outer retinal neuropathy, optic nerve neuritis, optic nerve degeneration associated with multiple sclerosis, Kjer's optic neuropathy, ischemic optic neuropathy, chemotherapy-induced peripheral neuropathy, neuromyelitis optica, Charcot Marie Tooth disease, deficiency in vitamin B12, deficiency in folic acid (vitamin B9), isolated vitamin E deficiency syndrome, non-arteritic anterior ischemic optic neuropathy, exposure to ethambutol, exposure to cyanide, traumatic brain injury (TBI), spinal cord injury, traumatic axonal injury or chronic traumatic encephalopathy (CTE).

42. The method of claim 40, wherein the SARM1-mediated neurological disease is amyotrophic lateral sclerosis, multiple sclerosis, chemotherapy-induced peripheral neuropathy (CIPN), diabetic peripheral neuropathy (DPN), tauopathy, or Charcot Marie Tooth disease.

43. The method of claim 40, wherein the SARM1-mediated neurological disease is amyotrophic lateral sclerosis.

44. The method of claim 40, wherein the SARM1 RNAi agent is administered to the patient intrathecally, intracerebroventricularly, or via intracisternal magna injection.

45. A method of reducing SARM1 expression in a cell, the method comprising:
(a) introducing the SARM1 RNAi agent of claim 1 into the cell; and
(b) incubating the cell for a time sufficient for degradation of SARM1 mRNA, thereby reducing SARM1 expression in the cell.

46.-57. (canceled)

58. A pharmaceutical composition comprising the SARM1 RNAi agent of claim 30 and a pharmaceutically acceptable carrier.

59. A method of reducing SARM1 expression in a patient in need thereof, the method comprising administering to the patient an effective amount of the SARM1 RNAi agent of claim 30.

60. A method of reducing axon degeneration in a patient in need thereof, the method comprising administering to the patient an effective amount of the SARM1 RNAi agent of claim 30.

61. A method of treating a SARM1-mediated neurological disease in a patient in need thereof, the method comprising administering to the patient an effective amount of the SARM1 RNAi agent of claim 30.

62. The method of claim 61, wherein the SARM1-mediated neurological disease is selected from amyotrophic lateral sclerosis (ALS, or Lou Gehrig's disease), Alzheimer's disease, Parkinson's disease, multiple sclerosis (MS), Huntington's disease (HD), senile dementia, Pick's disease, Gaucher's disease, Hurler syndrome, progressive multifocal leukoencephalopathy, Alexander's disease, congenital hypomyelination, encephalomyelitis, acute disseminated encephalomyelitis, central pontine myelinolysis, osmotic hyponatremia, Tay-Sachs disease, motor neuron disease, ataxia, spinal muscular atrophy (SMA), Niemann-Pick disease, acute hemorrhagic leukoencephalitis, trigeminal neuralgia, Bell's palsy, cerebral ischemia, multiple system atrophy, Pelizaeus Merzbacher disease, periventricular leukomalacia, a hereditary ataxia, noise-induced hearing loss, congenital hearing loss, age-related hearing loss, Creutzfeldt-Jakob disease, transmissible spongiform encephalopathy, Lewy Body Dementia, frontotemporal dementia, tauopathy, synucleinopathy, amyloidosis, diabetic neuropathy, globoid cell leukodystrophy (Krabbe's disease), Bassen-Komzweig syndrome, transverse myelitis, motor neuron disease, spinocerebellar ataxia, pre-eclampsia, hereditary spastic paraplegias, spastic paraparesis, familial spastic paraplegia, French settlement disease, Strumpell-Lorrain disease, non-alcoholic steatohepatitis (NASH), adrenomyeloneuropathy, progressive supra nuclear palsy (PSP), Friedrich's ataxia, spinal cord injury, acute optic neuropathy (AON), a genetic or idiopathic retinal condition, Leber congenital amaurosis (LCA), Leber hereditary optic neuropathy (LHON), primary open-angle glaucoma (POAG), acute angle-closure glaucoma (AACG), autosomal dominant optic atrophy, retinal ganglion degeneration, retinitis pigmentosa, an outer retinal neuropathy, optic nerve neuritis, optic nerve degeneration associated with multiple sclerosis, Kjer's optic neuropathy, ischemic optic neuropathy, chemotherapy-induced peripheral neuropathy, neuromyelitis optica, Charcot Marie Tooth disease, deficiency in vitamin B12, deficiency in folic acid (vitamin B9), isolated vitamin E deficiency syndrome, non-arteritic anterior ischemic optic neuropathy, exposure to ethambutol, exposure to cyanide, traumatic brain injury (TBI), spinal cord injury, traumatic axonal injury or chronic traumatic encephalopathy (CTE).
